(19) United States
(12) Patent Application Publication
(10) Pub. No.: US 2016/0237067 A1
(43) Pub. Date: Aug. 18, 2016

(54) SUBSTITUTED PHENYLALANINE DERIVATIVES

(71) Applicant: BAYER PHARMA AKTIENGESELLSCHAFT, Berlin (DE)

(72) Inventors: Ulrike RÖHN, Berlin (DE); Manuel ELLERMANN, Berlin (DE); Julia STRASSBURGER, Wuppertal (DE); Astrid WENDT, Syke (DE); Susanne RÖHRIG, Hilden (DE); Robert Alan WEBSTER, Wuppertal (DE); Martina Victoria SCHMIDT, Köln (DE); Adrian TERSTEEGEN, Wuppertal (DE); Kristin BEYER, Kensington, CA (US); Martina SCHÄFER, Berlin (DE); Anja BUCHMÜLLER, Essen (DE); Christoph GERDES, Köln (DE); Michael SPERZEL, Kierspe (DE); Steffen SANDMANN, Essen-Heisingen (DE); Stefan HEITMEIER, Wülfrath (DE); Alexander HILLISCH, Solingen (DE); Jens ACKERSTAFF, Düsseldorf (DE); Carsten TERJUNG, Bochum (DE)

(73) Assignee: Bayer Pharma Aktiengesellschaft, Berlin (DE)

(21) Appl. No.: 15/024,327

(22) PCT Filed: Sep. 24, 2014
(86) PCT No.: PCT/EP2014/070315

§ 371 Date: Mar. 24, 2016

(30) Foreign Application Priority Data
Sep. 26, 2013 (EP) 13186058.7

(51) Int. Cl. C07D 405/12; C07D 403/12; C07D 231/56; C07D 235/26; C07D 249/08; C07D 257/04; C07D 401/12

(52) U.S. Cl. CPC C07D 405/12; C07D 257/04; C07D 403/12; C07D 401/12; C07D 235/26; C07D 249/08; C07D 231/56

(57) ABSTRACT

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood.

In the more recent past, the traditional theory of two separate regions of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate is low and as a result of the occurrence of TFPI as inhibitor of the TF-FVIIa-FX complex is limited in time.

A central component of the transition from initiation to amplification and propagation of coagulation is factor XIa. In positive feedback loops, thrombin activates, in addition to factor V and factor VIII, also factor XI to factor XIa, whereby factor IX is converted into factor IXa, thus, via the factor IXa/factor Villa complex generated in this manner, rapidly producing relatively large amounts of factor Xa. This triggers the production of large amounts of thrombin, leading to strong thrombus growth and stabilizing the thrombus.

The formation of a thrombus or blood clot is counter-regulated by fibrinolysis. Activation of plasminogen by tissue plasminogen activator (tPA) results in formation of the active serine protease, plasmin, which cleaves polymerized fibrin and thus forms the thrombus. This process is referred to as fibrinolysis—with plasmin as key enzyme.

Uncontrolled activation of the coagulation system or defects in the inhibition of the activation processes may cause formation of local thromboses or embolisms in vessels (arteries, veins, lymph vessels) or heart chambers. This may lead to serious thrombotic or thromboembolic disorders. In addition, systemic hypercoagulability may lead to consumption coagulopathy in the context of a disseminated intravasal coagulation.

In the course of many cardiovascular and metabolic disorders, there is an increased tendency for coagulation and platelet activation owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis. This unwanted and excessive haemostasis may, by formation of fibrin- and platelet-rich thrombi, lead to thromboembolic disorders and thrombotic complications with life-threatening conditions.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants known from the prior art, for example substances for inhibiting or preventing blood coagulation, have various, frequently grave disadvantages. Accordingly, in practice, efficient treatment methods or the prophylaxis of thrombotic/thromboembolic disorders are found to be very difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis [Pschyrembel, Klinisches Wörterbuch [clinical dictionary], 257th edition, 1994, Walter de Gruyter Verlag, page 610, keyword “Heparin”; Römpp Lexikon Chemie, Version 1.5, 1998, Georg Thieme Verlag Stuttgart, keyword “Heparin”]. Low-molecular-weight heparins do have a lower probability of leading to the development of heparin-induced thrombocytopaenia; however, they can also only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of certain vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use, but they have also shown disadvantages, for example highly variable bioavailability, liver damage and bleeding complications.

For antithrombotic medicaments, the therapeutic width is of central importance: The interval between the therapeutically active dose for coagulation inhibition and the dose where bleeding may occur should be as large as possible so that maximum therapeutic activity is achieved at a minimum risk profile.

In various in vivo models with, for example, antibodies as factor XIa inhibitors, but also in factor XIa knock-out models, the antithrombotic effect with small/no prolongation of bleeding time or extension of blood volume was confirmed. In clinical studies, elevated factor XIa concentrations were associated with an increased event rate. However, factor XI deficiency (haemophilia C), in contrast to factor VIIIa or factor IXa (haemophilia A and B, respectively), did not lead to spontaneous bleeding and was only noticed during surgical interventions and traumata. Instead, protection against certain thromboembolic events was found.

In the event of hyperfibrinolytic states, there is inadequate wound closure, which causes severe, sometimes life-threatening, bleeding. This bleeding can be stopped by the inhibition of fibrinolysis with antifibrinolytics, by which plasmin activity is reduced. Corresponding effects with the plasminogen inhibitor tranexamic acid have been shown in various clinical studies.

It is therefore an object of the present invention to provide novel compounds for treatment and/or prophylaxis of cardiovascular disorders and/or severe perioperative blood loss in man and animals, said compounds having a wide therapeutic range.

WO89/11852 describes, inter alia, substituted phenylalanine derivatives for treatment of pancreatitis, and WO 2007/070816 describes substituted thiophene derivatives as factor XIa inhibitors.

The invention provides compounds of the formula

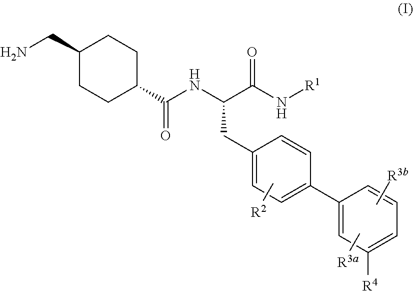

in which
R¹ represents a group of the formula

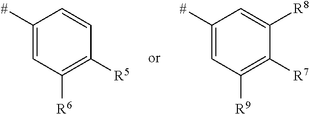

where # is the point of attachment to the nitrogen atom,
R⁵ represents 5-membered heteroaryl,
   where heteroaryl may be substituted by a substituent selected from the group consisting of oxo, chlorine, cyano, hydroxyl and C₁-C₃-alkyl,
      in which alkyl may be substituted by 1 to 3 substituents selected independently from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R⁶ represents hydrogen, fluorine or chlorine,
R⁷ and R⁸ together with the carbon atoms to which they are attached form a 5-membered heterocycle,
   where the heterocycle may be substituted by 1 to 2 substituents selected independently from the group consisting of oxo, chlorine, cyano, hydroxyl, C₁-C₃-alkyl, pyrazolyl and pyridyl,
      in which alkyl may be substituted by 1 to 3 substituents selected independently from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R⁹ represents hydrogen, fluorine or chlorine,
R² represents hydrogen, fluorine, chlorine, methyl or methoxy,
R^{3a} represents hydrogen, fluorine, chlorine, C₁-C₄-alkyl, methoxy or trifluoromethyl,
R^{3b} represents hydrogen or methyl,
R⁴ represents amino, cyano, trifluoromethyl, C₁-C₃-alkoxy, C₁-C₃-alkylamino, C₁-C₃-alkoxycarbonyl, —S(O)₂NR¹⁰R¹¹, —C(O)NR¹²R¹³ or —NR¹⁴(CO)R¹⁵,
   where alkoxy is substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
      in which n is a number from 1 to 6,
      in which m is a number from 1 to 6,
   and
   where
   R¹⁰ represents hydrogen, C₁-C₃-alkyl, C₃-C₆-cycloalkyl, benzyl or 4- to 8-membered heterocyclyl which is attached via a carbon atom,
   R¹¹ represents hydrogen or C₁-C₃-alkyl,
   or
   R¹⁰ and R¹¹ together with the nitrogen atom to which they are attached form a 4- to 7-membered heterocycle,
      in which the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxy carbonyl, aminocarbonyl and C₁-C₃-alkylaminocarbonyl,
   R¹² represents hydrogen, C₁-C₃-alkyl, C₁-C₃-alkoxy, C₃-C₆-cycloalkyl, benzyl or 4- to 8-membered heterocyclyl which is attached via a carbon atom,
      in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
         in which n is a number from 1 to 6,
         in which m is a number from 1 to 6,
   and
      in which cycloalkyl may be substituted by 1 to 2 substituents independently of one another selected from the group consisting of oxo, fluorine, hydroxy, amino, C₁-C₄-alkyl and C₁-C₃-alkylamino,
         in which alkyl and alkylamino for their part may be substituted by 1 to 5 fluorine substituents,
      and
      in which heterocyclyl may be substituted by 1 to 2 substituents independently of one another selected from the group consisting of oxo, fluorine, hydroxy, amino, hydroxy carbonyl, C₁-C₄-alkyl, C₁-C₃-alkylamino, C₁-C₄-alkoxy carbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
   R¹³ represents hydrogen or C₁-C₃-alkyl,
   or
   R¹² and R¹³ together with the nitrogen atom to which they are attached form a 4- to 7-membered heterocycle,
      in which the heterocycle may be substituted by 1 to 2 substituents selected independently from the group consisting of oxo, fluorine, hydroxyl, amino, hydroxy carbonyl, C₁-C₄-alkyl, C₁-C₃-alkylamino, difluoromethyl, trifluoromethyl, 2,2,2-trifluoroeth-1-yl, C₁-C₄-alkoxy carbonyl, aminocarbonyl and C₁-C₃-alkylaminocarbonyl,
         in which alkyl for its part may be substituted by a hydroxy substituent,
   R¹⁴ represents hydrogen or C₁-C₃-alkyl,
   R¹⁵ represents C₁-C₄-alkyl, C₃-C₆-cycloalkyl, phenyl or 5- to 7-membered heterocyclyl,
      and the salts thereof, the solvates thereof and the solvates of the salts thereof.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The compounds of the invention may, depending on their structure, exist in different stereoisomeric forms, i.e. in the form of configurational isomers or else, if appropriate, of conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I, and ¹³¹I. Particular isotopic variants of a compound of the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting compounds.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the compounds according to the invention which form a complex in the solid or liquid state by coordination with solvent molecules. Hydrates are a specific form of the solvates in which the coordination is with water.

The present invention additionally also encompasses prodrugs of the compounds of the invention. The term “prodrugs” encompasses compounds which for their part may be biologically active or inactive but are converted during their residence time in the body into compounds according to the invention (for example by metabolism or hydrolysis).

The two ways (A) and (B) of representing a 1,4-disubstituted cyclohexyl derivative shown below are equivalent to one another and identical, and in both cases describe a trans-1,4-disubstituted cyclohexyl derivative.

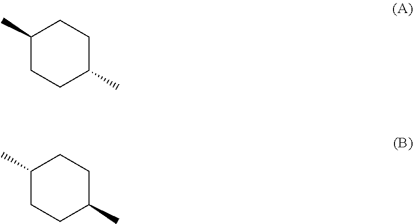

This applies especially to the structural element of tranexamamide, for example N-[(trans-4-{[(tert-butoxycarbonyl)amino]methyl}cyclohexyl)carbonyl and trans-4-(aminomethyl)-cyclohexyl]carbonyl}. This also applies to the structural element of trans-4-hydroxycyclohexylamine, for example in (trans-4-hydroxycyclohexyl)carbamoyl. In the present invention, representation (A) is used with preference for tranexamamide.

The three ways (C), (D) and (E) of representing tautomers of a triazole derivative shown below are equivalent to one another and identical and in all cases describe a 1,4-disubstituted triazole derivative.

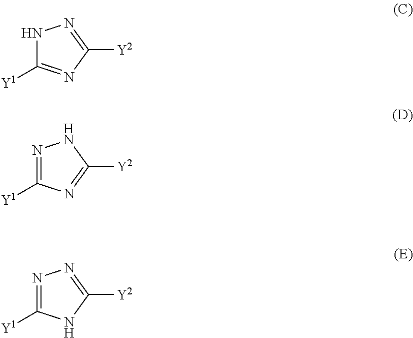

This applies especially to the following structural elements: 1H-1,2,4-triazol-3-yl, 1H-1,2,4-triazol-5-yl, 4H-1,2,4-triazol-3-yl and 4H-1,2,4-triazol-5-yl. Y¹ and Y² here are different substituents.

The two ways (F) and (G) of representing tautomers of a tetrazole derivative shown below are equivalent to one another and identical and in all cases describe a tetrazole derivative.

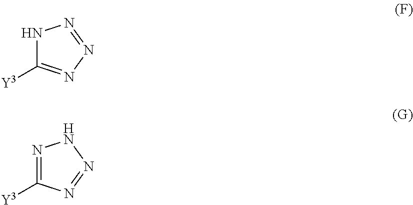

This applies especially to the following structural elements: 1H-tetrazol-5-yl and 2H-tetrazol-5-yl. Y³ here is the remainder of the compound.

The compounds according to the invention of the formula

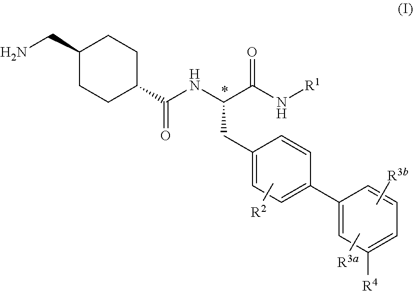

and all L-phenylalanine intermediates are described as the (S) configuration at the stereocentre marked with an * in the above formula, since L-phenylalanine derivatives are introduced into the synthesis as central units. In the preparation of the compounds according to the invention, the coupling of the L-phenylalanine intermediates with the amine H₂N—R¹ can result in partial epimerization at the stereocentre marked with an *. Thus, a mixture of the compounds according to the invention of (S) enantiomer and (R) enantiomer can arise. The main component is the (S) enantiomer depicted in each case. The mixtures of (S) enantiomer and (R) enantiomer can be separated into their enantiomers by methods known to those skilled in the art, for example by chromatography on a chiral phase.

The enantiomers can be separated either directly after the coupling of the L-phenylalanine intermediates with the amine H₂N—R¹ or at a later synthesis intermediate, or else the inventive compounds can be separated themselves. Preference is given to the separation of the enantiomers directly after the coupling of the L-phenylalanine intermediates with the amine H₂N—R¹.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is used here synonymously with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, unless specified otherwise, the substituents are defined as follows:

Alkyl represents a straight-chain or branched alkyl radical having 1 to 4 carbon atoms, preferably 1 to 3 carbon atoms, by way of example and with preference methyl, ethyl, n-propyl, isopropyl, 2-methylprop-1-yl, n-butyl and tert-butyl.

Alkoxy represents a straight-chain or branched alkoxy radical having 1 to 4 carbon atoms, preferably 1 to 3 carbon atoms, by way of example and with preference methoxy, ethoxy, n-propoxy, isopropoxy, 2-methylprop-1-oxy, n-butoxy and tert-butoxy.

Alkylamino represents an amino group having one or two independently selected, identical or different, straight-chain or branched alkyl radicals each having 1 to 3 carbon atoms, for example and with preference methylamino, ethylamino, n-propylamino, isopropylamino, N,N-dimethylamino, N,N-diethylamino, N-ethyl-N-methylamino, N-methyl-N-n-propylamino, N-isopropyl-N-n-propylamino and N,N-diisopropylamino. C₁-C₃-Alkylamino represents, for example, a monoalkylamino radical having 1 to 3 carbon atoms or a dialkylamino radical having 1 to 3 carbon atoms in each alkyl radical.

Alkoxycarbonyl represents a straight-chain or branched alkoxy radical attached via a carbonyl group and having 1 to 4 carbon atoms, preferably 1 to 3 carbon atoms, for example and with preference methoxycarbonyl, ethoxycarbonyl, n-propoxycarbonyl, isopropoxycarbonyl, n-butoxycarbonyl and tert-butoxycarbonyl.

Alkylaminocarbonyl represents an amino group having one or two independently selected, identical or different, straight-chain or branched alkyl substituents each having 1 to 3 carbon atoms, attached via a carbonyl group, for example and with preference methylaminocarbonyl, ethylaminocarbonyl, n-propylaminocarbonyl, isopropylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N,N-diisopropylaminocarbonyl. C₁-C₃-Alkylaminocarbonyl represents, for example, a monoalkylaminocarbonyl radical having 1 to 3 carbon atoms or a dialkylaminocarbonyl radical having 1 to 3 carbon atoms in each alkyl substituent.

Cycloalkyl represents a monocyclic cycloalkyl group having 3 to 6 carbon atoms, preferred examples of cycloalkyl being cyclopropyl, cyclobutyl, cyclopentyl and cyclohexyl.

4- to 8-membered heterocyclyl attached via a carbon atom in the definition of the radicals R¹⁰ and R¹² represents a saturated or partially unsaturated monocyclic or bicyclic radical which is attached via a carbon atom and which has 4 to 8 ring atoms, preferably 5 or 6 ring atoms, and up to 3 heteroatoms and/or hetero groups, preferably 1 or 2 heteroatoms and/or hetero groups from the series S, O, N, SO and SO₂, where a nitrogen atom may also form an N-oxide, for example and with preference azetidinyl, pyrrolidinyl, piperidinyl, tetrahydropranyl, 3-azabicyclo[3.1.0]hex-6-yl, 8-azabicyclo[3.2.1]oct-3-yl and azepanyl, particularly preferably pyrrolidinyl and piperidinyl.

4- to 7-membered heterocycle in the definition of the R¹⁰ and R¹¹ radicals and the radicals R¹² and R¹³ represents a saturated or partially unsaturated, monocyclic or bicyclic radical having 4 to 7 ring atoms, preferably 5 or 6 ring atoms, and up to 3 heteroatoms and/or hetero groups, preferably 1 or 2 heteroatoms and/or hetero groups, from the group of S, O, N, SO and SO₂, where one nitrogen atom may also form an N-oxide, for example and with preference azetidinyl, pyrrolidinyl, morpholinyl, thiomorpholinyl, piperidinyl, piperazinyl, 3-azabicyclo[3.1.0]hex-6-yl, 8-azabicyclo[3.2.1]oct-3-yl and azepanyl, particularly preferably morpholinyl and piperazinyl.

5-membered heteroaryl in the definition of the R⁵ radical represents an aromatic monocyclic radical having 5 ring atoms and up to 4 heteroatoms and/or hetero groups from the group consisting of S, O, N, SO and SO₂, where one nitrogen atom may also form an N-oxide, for example and with preference thienyl, furyl, pyrrolyl, thiazolyl, oxazolyl, isoxazolyl, oxadiazolyl, pyrazolyl, imidazolyl, triazolyl and tetrazolyl, particularly preferably triazolyl and tetrazolyl.

5-membered heterocycle in the definition of the R⁷ and R⁸ radicals represents a saturated, partially unsaturated or aromatic monocyclic radical having 5 ring atoms and up to 2 heteroatoms and/or hetero groups from the group of S, O, N, SO and SO₂, where one nitrogen atom may also form an N-oxide. This 5-membered heterocycle together with the phenyl ring to which it is attached represents, for example and with preference, 2,3-dihydro-1-benzothiophen-5-yl, 1,3-dihydro-2-benzothiophen-5-yl, 2,3-dihydro-1-benzofuran-5-yl, 1,3-dihydro-2-benzofuran-5-yl, indolin-5-yl, isoindolin-5-yl, 2,3-dihydro-1H-indazol-5-yl, 2,3-dihydro-1H-benzimidazol-5-yl, 1,3-dihydro-2, 1-benzoxazol-5-yl, 2,3-dihydro-1,3-benzoxazol-5-yl, 1,3-dihydro-2, 1-benzothiazol-5-yl, 2,3-dihydro-1,3-benzothiazol-5-yl, 1H-benzimidazol-5-yl, 1H-indazol-5-yl, 1,2-benzoxazol-5-yl, indol-5-yl, isoindol-5-yl, benzofuran-5-yl, benzothiophen-5-yl, 2,3-dihydro-1-benzothiophen-6-yl, 1,3-dihydro-2-benzothiophen-6-yl, 2,3-dihydro-1-benzofuran-6-yl, 1,3-dihydro-2-benzofuran-6-yl, indolin-6-yl, isoindolin-6-yl, 2,3-dihydro-1H-indazol-6-yl, 2,3-dihydro-1H-benzimidazol-6-yl, 1,3-dihydro-2,1-benzoxazol-6-yl, 2,3-dihydro-1,3-benzoxazol-6-yl, 1,3-dihydro-2, 1-benzothiazol-6-yl, 2,3-dihydro-1,3-benzothiazol-6-yl, 1H-benzimidazol-6-yl, 1H-indazol-6-yl, 1,2-benzoxazol-6-yl, indol-6-yl, isoindol-6-yl, benzofuran-6-yl and benzothiophen-6-yl, particularly preferably 2,3-dihydro-1H-indazol-6-yl and 1H-benzimidazol-6-yl, very particularly preferably 2,3-dihydro-1H-indazol-6-yl.

5- to 7-membered heterocyclyl in the definition of the radical R¹⁵ represents a saturated or partially unsaturated, monocyclic or bicyclic radical having 5 to 7 ring atoms, preferably 5 or 6 ring atoms, and up to 3 heteroatoms and/or hetero groups, preferably 1 or 2 heteroatoms and/or hetero groups, from the group consisting of S, O, N, SO and SO₂, where one nitrogen atom may also form an N-oxide, for example and with preference pyrrolidinyl, morpholinyl, thiomorpholinyl, piperidinyl, piperazinyl, tetrahydropyranyl, 3-azabicyclo[3.1.0]hex-6-yl, 8-azabicyclo[3.2.1]oct-3-yl and azepanyl, particularly preferably tetrahydropyranyl.

In the formulae of the group which may represent R¹, the end point of the line marked by # in each case does not represent a carbon atom or a CH₂ group, but is part of the bond to the atom to which R¹ is attached.

Preference is given to compounds of the formula (I) in which
R¹ represents a group of the formula

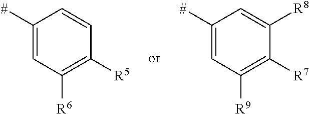

where # is the point of attachment to the nitrogen atom,
R⁵ represents 5-membered heteroaryl,
   where heteroaryl may be substituted by a substituent selected from the group consisting of oxo, chlorine, cyano, hydroxyl and C₁-C₃-alkyl,
      in which alkyl may be substituted by 1 to 3 substituents selected independently from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R⁶ represents hydrogen, fluorine or chlorine,
R⁷ and R⁸ together with the carbon atoms to which they are attached form a 5-membered heterocycle,
   where the heterocycle may be substituted by 1 to 2 substituents selected independently from the group consisting of oxo, chlorine, cyano, hydroxyl, C₁-C₃-alkyl, pyrazolyl and pyridyl,
      in which alkyl may be substituted by 1 to 3 substituents selected independently from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R⁹ represents hydrogen, fluorine or chlorine,
R² represents hydrogen, fluorine, chlorine, methyl or methoxy,
R^{3a} represents hydrogen, fluorine, chlorine, C₁-C₄-alkyl, methoxy or trifluoromethyl,
R^{3b} represents hydrogen,
R⁴ represents amino, cyano, trifluoromethyl, C₁-C₃-alkoxy, C₁-C₃-alkylamino, C₁-C₃-alkoxycarbonyl, —S(O)₂NR¹⁰R¹¹, —C(O)NR¹²R¹³ or —NR¹⁴(CO)R¹⁵,
   where alkoxy is substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
      in which n is a number from 1 to 6,
      in which m is a number from 1 to 6,
   and
   where
   R¹⁰ represents hydrogen, C₁-C₃-alkyl, C₃-C₆-cycloalkyl, benzyl or 4- to 8-membered heterocyclyl which is attached via a carbon atom,
   R¹¹ represents hydrogen or C₁-C₃-alkyl,
   or
   R¹⁰ and R¹¹ together with the nitrogen atom to which they are attached form a 4- to 7-membered heterocycle,
      in which the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxy carbonyl, aminocarbonyl and C₁-C₃-alkylaminocarbonyl,
   R¹² represents hydrogen, C₁-C₃-alkyl, C₁-C₃-alkoxy, C₃-C₆-cycloalkyl, benzyl or 4- to 8-membered heterocyclyl which is attached via a carbon atom,
      in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
         in which n is a number from 1 to 6,
         in which m is a number from 1 to 6,
   and
      in which cycloalkyl may be substituted by 1 to 2 substituents independently of one another selected from the group consisting of oxo, fluorine, hydroxy, amino, C₁-C₄-alkyl and C₁-C₃-alkylamino,
         in which alkyl and alkylamino for their part may be substituted by 1 to 5 fluorine substituents,
      and
      in which heterocyclyl may be substituted by 1 to 2 substituents independently of one another selected from the group consisting of oxo, fluorine, hydroxy, amino, hydroxy carbonyl, C₁-C₄-alkyl, C₁-C₃-alkylamino, C₁-C₄-alkoxy carbonyl, aminocarbonyl and C₁-C₃-alkylaminocarbonyl,
         in which alkyl and alkylamino for their part may be substituted by 1 to 5 fluorine substituents,
      and in which heterocyclyl may additionally be substituted by 1 to 4 substituents independently of one another selected from the group consisting of fluorine and methyl,
   R¹³ represents hydrogen or C₁-C₃-alkyl,
   or
   R¹² and R¹³ together with the nitrogen atom to which they are attached form a 4- to 7-membered heterocycle,
      in which the heterocycle may be substituted by 1 to 2 substituents selected independently from the group consisting of oxo, fluorine, hydroxyl, amino, hydroxy carbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
         in which alkyl for its part may be substituted by a hydroxy substituent,
   R¹⁴ represents hydrogen or C₁-C₃-alkyl,
   R¹⁵ represents C₁-C₄-alkyl, C₃-C₆-cycloalkyl, phenyl or 5- to 7-membered heterocyclyl,
      and the salts thereof, the solvates thereof and the solvates of the salts thereof.

Preference is also given to compounds of the formula (I) in which
R¹ represents a group of the formula

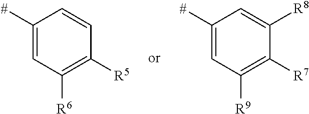

where # is the point of attachment to the nitrogen atom,
R⁵ represents 5-membered heteroaryl,
   where heteroaryl may be substituted by a substituent selected from the group consisting of oxo, chlorine and C₁-C₃-alkyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁶ represents hydrogen or fluorine,
R⁷ and R⁸ together with the carbon atoms to which they are attached form a 5-membered heterocycle,
   where the heterocycle may be substituted by 1 to 2 substituents selected independently from the group consisting of oxo, chlorine, hydroxyl, C₁-C₃-alkyl, pyrazolyl and pyridyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁹ represents hydrogen or fluorine,
R² represents hydrogen, fluorine, methyl or methoxy,
R^{3a} represents hydrogen, fluorine, chlorine, C₁-C₄-alkyl, methoxy or trifluoromethyl,
R^{3b} represents hydrogen,
R⁴ represents amino, cyano, trifluoromethyl, C₁-C₃-alkoxy, C₁-C₃-alkylamino, C₁-C₃-alkoxycarbonyl, —S(O)₂NR¹⁰R¹¹, —C(O)NR¹²R¹³ or —NR¹⁴(CO)R¹⁵,
   where alkoxy is substituted by a morpholinyl substituent,
   and
   where
   R¹⁰ represents hydrogen, methyl, ethyl, C₃-C₆-cycloalkyl or 4- to 8-membered heterocyclyl which is attached via a carbon atom,
   R¹¹ represents hydrogen, methyl or ethyl,
   or
   R¹⁰ and R¹¹ together with the nitrogen atom to which they are attached form a 4- to 7-membered heterocycle,
      in which the heterocycle may be substituted by 1 to 2 substituents independently of one another selected from the group consisting of C₁-C₄-alkyl,
   R¹² is hydrogen, C₁-C₃-alkyl, C₃-C₆-cycloalkyl, or 4- to 8-membered heterocyclyl bonded via a carbon atom,
      in which alkyl may be substituted by 1 to 2 substituents independently of one another selected from the group consisting of C₁-C₃-alkylamino and morpholinyl,
      and
      in which cycloalkyl may be substituted by 1 to 2 substituents independently of one another selected from the group consisting of hydroxy, amino, C₁-C₄-alkyl and C₁-C₃-alkylamino,
      and
      in which heterocyclyl may be substituted by 1 to 2 substituents independently of one another selected from the group consisting of C₁-C₄-alkyl,
   R¹³ represents hydrogen, methyl or ethyl,
   or
   R¹² and R¹³ together with the nitrogen atom to which they are attached form a 4- to 7-membered heterocycle,
      in which the heterocycle may be substituted by 1 to 2 substituents independently of one another selected from the group consisting of C₁-C₄-alkyl,
   R¹⁴ represents hydrogen, methyl or ethyl,
   R¹⁵ represents C₁-C₄-alkyl or 5- to 7-membered heterocyclyl,
      and the salts thereof, the solvates thereof and the solvates of the salts thereof.

Preference is also given to compounds of the formula (I) in which
R¹ represents a group of the formula

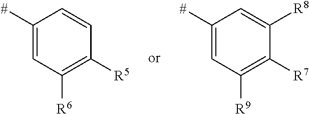

where # is the point of attachment to the nitrogen atom,
R⁵ represents 5-membered heteroaryl,
   where heteroaryl may be substituted by a chlorine substituent,
R⁶ represents hydrogen,
R⁷ and R⁸ together with the carbon atoms to which they are attached form a 5-membered heterocycle,
   where the heterocycle may be substituted by an oxo substituent,
R⁹ represents hydrogen,
R² represents hydrogen,
R^{3a} represents hydrogen, fluorine, C₁-C₄-alkyl or methoxy,
R^{3b} represents hydrogen,
R⁴ represents cyano, trifluoromethyl, C₁-C₃-alkoxy, C₁-C₃-alkylamino, C₁-C₃-alkoxycarbonyl, —S(O)₂NR¹⁰R¹¹, —C(O)NR¹²R¹³ or —NR¹⁴(CO)R¹⁵,
   where alkoxy is substituted by a morpholinyl substituent,
   and
   where
   R¹⁰ represents hydrogen or methyl,
   R¹¹ represents hydrogen or methyl,
   or
   R¹⁰ and R¹¹ together with the nitrogen atom to which they are attached form a 4- to 7-membered heterocycle,
   R¹² represents C₁-C₃-alkyl or 4- to 8-membered heterocyclyl which is attached via a carbon atom,
      in which alkyl may be substituted by 1 to 2 substituents independently of one another selected from the group consisting of C₁-C₃-alkylamino and morpholinyl,
   R¹³ represents hydrogen,
   or
   R¹² and R¹³ together with the nitrogen atom to which they are attached form a 4- to 7-membered heterocycle,
      in which the heterocycle may be substituted by 1 to 2 methyl substituents,
   R¹⁴ represents hydrogen,
   R¹⁵ represents 5- to 7-membered heterocyclyl,
      and the salts thereof, the solvates thereof and the solvates of the salts thereof.

Preference is also given to compounds of the formula (I) in which
R¹ represents a group of the formula

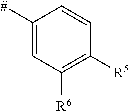

where # is the point of attachment to the nitrogen atom,
R⁵ is triazolyl or tetrazolyl,
   where triazolyl may be substituted by a chlorine substituent,
R⁶ represents hydrogen,
or
R¹ represents 2,3-dihydro-1H-indazol-6-yl,
   where 2,3-dihydro-1H-indazol-6-yl may be substituted by an oxo substituent,
R² represents hydrogen,
R^{3a} represents hydrogen, fluorine, C₁-C₄-alkyl or methoxy,
R^{3b} represents hydrogen,
R⁴ represents cyano, trifluoromethyl, C₁-C₃-alkoxycarbonyl, —S(O)₂NR¹⁰R¹¹ or —C(O)NR¹²R¹³,
   where
   R¹⁰ represents hydrogen or methyl,
   R¹¹ represents hydrogen or methyl,
   or
   R¹⁰ and R¹¹ together with the nitrogen atom to which they are attached form a morpholinyl or piperazinyl,
   R¹² represents C₁-C₃-alkyl or heterocyclyl which is attached via a carbon atom and selected from the group consisting of pyrrolidinyl and piperidinyl,
      in which alkyl may be substituted by 1 to 2 substituents independently of one another selected from the group consisting of C₁-C₃-alkylamino and morpholinyl,
   R¹³ represents hydrogen,
   or
   R¹² and R¹³ together with the nitrogen atom to which they are attached form a morpholinyl or piperazinyl,
      in which morpholinyl and piperazinyl may be substituted by 1 to 2 methyl substituents,
         and the salts thereof, the solvates thereof and the solvates of the salts thereof.

Preference is also given to compounds of the formula (I) in which
R¹ represents a group of the formula

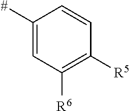

where # is the point of attachment to the nitrogen atom,
R⁵ is triazolyl or tetrazolyl,
   where triazolyl may be substituted by a chlorine substituent,
R⁶ represents hydrogen,
or
R¹ represents 2,3-dihydro-1H-indazol-6-yl or 2,3-dihydro-1H-benzimidazol-5-yl,
   where 2,3-dihydro-1H-indazol-6-yl and 2,3-dihydro-1H-benzimidazol-5-yl may be substituted by an oxo substituent.
R² represents hydrogen,
R^{3a} represents hydrogen, fluorine, C₁-C₄-alkyl or methoxy,
R^{3b} represents hydrogen,
R⁴ represents cyano, trifluoromethyl, C₁-C₃-alkoxycarbonyl, —S(O)₂NR¹⁰R¹¹ or —C(O)NR¹²R¹³,
   where
   R¹⁰ represents hydrogen or methyl,
   R¹¹ represents hydrogen or methyl,
   or
   R¹⁰ and R¹¹ together with the nitrogen atom to which they are attached form a morpholinyl or piperazinyl,
   R¹² represents C₁-C₃-alkyl or heterocyclyl which is attached via a carbon atom and selected from the group consisting of pyrrolidinyl and piperidinyl,
      in which alkyl may be substituted by 1 to 2 substituents independently of one another selected from the group consisting of C₁-C₃-alkylamino and morpholinyl,
   R¹³ represents hydrogen,
   or
   R¹² and R¹³ together with the nitrogen atom to which they are attached form a morpholinyl or piperazinyl,
      in which morpholinyl and piperazinyl may be substituted by 1 to 2 methyl substituents,
         and the salts thereof, the solvates thereof and the solvates of the salts thereof.

Preference is also given to compounds of the formula (I) in which
R¹ represents a group of the formula

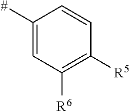

where # is the point of attachment to the nitrogen atom,
R⁵ is triazolyl or tetrazolyl,
   where triazolyl may be substituted by a chlorine substituent,
and
R⁶ represents hydrogen.

Preference is also given to compounds of the formula (I) in which
R¹ represents 2,3-dihydro-1H-indazol-6-yl,
   where 2,3-dihydro-1H-indazol-6-yl may be substituted by an oxo substituent.

Preference is also given to compounds of the formula (I) in which
R¹ represents 2,3-dihydro-1H-indazol-6-yl or 2,3-dihydro-1H-benzimidazol-5-yl,
   where 2,3-dihydro-1H-indazol-6-yl and 2,3-dihydro-1H-benzimidazol-5-yl may be substituted by an oxo substituent.

Preference is also given to compounds of the formula (I) in which R² represents hydrogen.

Preference is also given to compounds of the formula (I) in which R^{3a} represents hydrogen, fluorine, C₁-C₄-alkyl or methoxy.

Preference is also given to compounds of the formula (I) in which R^{3b} represents hydrogen.

Preference is also given to compounds of the formula (I) in which
R⁴ represents cyano, trifluoromethyl, C₁-C₃-alkoxycarbonyl, —S(O)₂NR¹⁰R¹¹ or —C(O)NR¹²R¹³,
   where
   R¹⁰ represents hydrogen or methyl,
   R¹¹ represents hydrogen or methyl,
   or
   R¹⁰ and R¹¹ together with the nitrogen atom to which they are attached form a morpholinyl or piperazinyl,
   R¹² represents C₁-C₃-alkyl or heterocyclyl which is attached via a carbon atom and selected from the group consisting of pyrrolidinyl and piperidinyl,
      in which alkyl may be substituted by 1 to 2 substituents independently of one another selected from the group consisting of C₁-C₃-alkylamino and morpholinyl,
   R¹³ represents hydrogen,
   or
   R¹² and R¹³ together with the nitrogen atom to which they are attached form a morpholinyl or piperazinyl,
      in which morpholinyl and piperazinyl may be substituted by 1 to 2 methyl substituents.

Irrespective of the particular combinations of the radicals specified, the individual radical definitions specified in the particular combinations or preferred combinations of radicals are also replaced as desired by radical definitions from other combinations.

Very particular preference is given to combinations of two or more of the abovementioned preferred ranges.

The invention further provides a process for preparing the compounds of the formula (I), or the salts thereof, solvates thereof and the solvates of the salts thereof, wherein the compounds of the formula

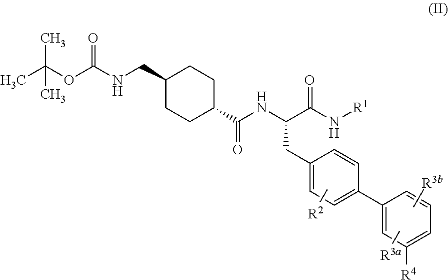

in which
R¹, R², R^{3a}, R^{3b} and R⁴ have the meaning given above,
are reacted with an acid.

The reaction is generally effected in inert solvents, preferably within a temperature range from room temperature to 60° C. at standard pressure.

Inert solvents are, for example, halogenated hydrocarbons such as dichloromethane, trichloromethane, tetrachloromethane or 1,2-dichloroethane, or ethers such as tetrahydrofuran or dioxane, preference being given to dioxane.

Acids are, for example, trifluoroacetic acid or hydrogen chloride in dioxane, preference being given to hydrogen chloride in dioxane.

The compounds of the formula (II) are known or can be prepared by

[A] reacting compounds of the formula

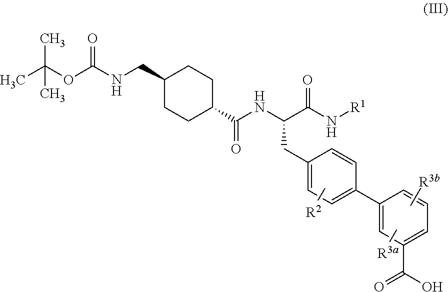

in which
R¹, R², R^{3a} and R^{3b} have the meaning given above,
with compounds of the formula

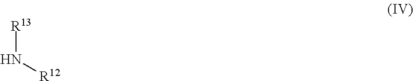

in which
R¹² and R¹³ have the meaning given above,
in the presence of a dehydrating reagent
or
[B] reacting compounds of the formula

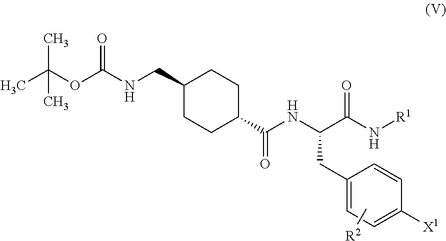

in which
R¹ and R² have the meaning given above, and
X¹ is bromine or iodine,
with compounds of the formula

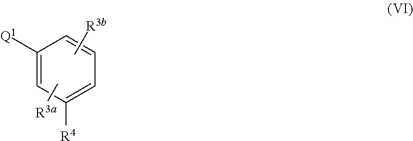

in which
R^{3a}, R^{3b} and R⁴ have the meaning given above and
Q¹ represents —B(OH)₂, a boronic ester, preferably pinacol boronate, or —BF₃⁻K⁺,
under Suzuki coupling conditions,
or
[C] reacting compounds of the formula

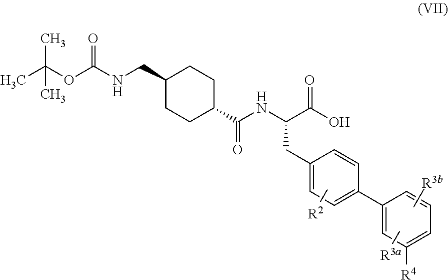

in which
R², R^{3a}, R^{3b} and R⁴ have the meaning given above,
with compounds of the formula

H₂N—R¹ (VIII)

in which
R¹ has the meaning given above,
in the presence of a dehydrating reagent.

The reaction in process [A] is generally effected in inert solvents, optionally in the presence of a base, preferably within a temperature range from 0° C. to the reflux of the solvents at standard pressure.

Suitable dehydrating agents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1, 1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or ethyl cyano(hydroxyimino)acetate (Oxyma), or (1-cyano-2-ethoxy-2-oxoethylideneaminooxy)dimethylaminomorpholinocarbenium hexafluorophosphate (COMU), or N-[(dimethylamino) (3H-[1,2,3]triazolo[4,5-b]pyridin-3-yloxy)methylidene]-N-methylmethanaminium hexafluorophosphate, or 2,4,6-tripropyl-1,3,5,2,4,6-trioxatriphosphinane 2,4,6-trioxide (T3P), or mixtures of these, with preference being given to N-[dimethylamino)(3H-[1,2,3]triazolo[4,5-b]pyridin-3-yloxy)methylidene]-N-methylmethanaminium hexafluorophosphate or 2,4,6-tripropyl-1,3,5,2,4,6-trioxatriphosphinane 2,4,6-trioxide (T3P).

Bases are, for example, alkali metal carbonates such as sodium carbonate or potassium carbonate, or sodium bicarbonate or potassium bicarbonate, or organic bases such as trialkylamines, for example triethylamine, N-methylmorpholine, N-methylpiperidine, 4-dimethylaminopyridine or diisopropylethylamine; preference is given to diisopropylethylamine.

Inert solvents are, for example, halogenated hydrocarbons such as dichloromethane or trichloromethane, hydrocarbons such as benzene, or other solvents such as nitromethane, tetrahydrofuran, dioxane, dimethylformamide, dimethyl sulphoxide, acetonitrile or pyridine, or mixtures of the solvents, preference being given to tetrahydrofuran or dimethylformamide or a mixture of dimethylformamide and pyridine.

The compounds of the formula (IV) are known, can be synthesized from the corresponding starting compounds by known processes or can be prepared analogously to the processes described in the Examples section.

The reaction in process [B] is generally effected in inert solvents, in the presence of a catalyst, optionally in the presence of an additional reagent, optionally in a microwave, preferably within a temperature range from room temperature to 150° C. at standard pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphaneferrocenyl)palladium (II) chloride, 1,3-bis(2,6-diisopropylphenyl) imidazol-2-ylidene (1,4-naphtho quinone)palladium dimer, allyl(chloro)(1,3-dimesityl-1,3-dihydro-2H-imidazol-2-ylidene)palladium, palladium(II) acetate/dicyclohexyl (2′,4′,6′-triisopropyl-biphenyl-2-yl)phosphine, [1, 1-bis(diphenylphosphino)ferrocene]palladium(II) chloride monodichloromethane adduct, XPhos precatalyst [(2′-aminobiphenyl-2-yl)(chloro)palladium dicyclohexyl(2′,4′,6′-triisopropylbiphenyl-2-yl)phosphane (1:1)] or [2′-(azanidyl-kappa-N-)biphenyl-2-yl-kappa[2][di-(3s,5s,7s)-adamantan-1-yl(buty)phosphoranyl]methanesulphonato-kappa-O-)palladium, or preference being given to tetrakistriphenylphosphinepalladium(0), [1,1-bis-(diphenylphosphino)ferrocene]palladium (II) chloride monodichloromethane adduct, XPhos precatalyst [(2′-aminobiphenyl-2-yl)(chloro)palladium dicyclohexyl(2′,4′,6′-triisopropylbiphenyl-2-yl)phosphane (1:1)].

Additional reagents are, for example, potassium acetate, caesium carbonate, potassium carbonate or sodium carbonate, potassium tert-butoxide, caesium fluoride or potassium phosphate, which may be present in aqueous solution; preferred additional reagents are those such as potassium acetate or a mixture of potassium acetate and sodium carbonate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, or mixtures of the solvents with alcohols such as methanol or ethanol and/or water, preference being given to toluene, dimethylformamide or dimethyl sulphoxide.

The compounds of the formula (VI) are known, can be synthesized from the corresponding starting compounds by known processes or can be prepared analogously to the processes described in the Examples section.

The reaction in process [C] is effected as described for process [A].

The compounds of the formula (VIII) are known, can be synthesized from the corresponding starting compounds by known processes or can be prepared analogously to the processes described in the Examples section.

The compounds of the formula (III) are known or can be prepared by

[D] reacting compounds of the formula

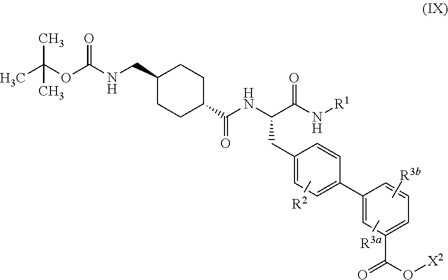

in which
R¹, R², R^{3a} and R^{3b} have the meaning given above, and
X² represents methyl or ethyl,
with a base,
or
[E] reacting compounds of the formula (V) with compounds of the formula

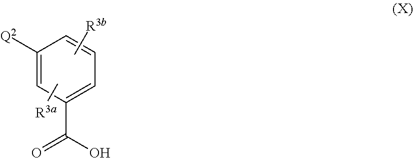

in which
R^{3a} and R^{3b} have the meaning given above, and
Q² represents —B(OH)₂, a boronic ester, preferably pinacol boronate, or —BF₃⁻K⁺,
under Suzuki coupling conditions.

The reaction in process [D] is generally carried out in inert solvents, preferably within a temperature range from room temperature up to the reflux of the solvents at standard pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvents with water, preference being given to a mixture of tetrahydrofuran and water.

Bases are, for example, alkali metal hydroxides such as sodium hydroxide, lithium hydroxide or potassium hydroxide, or alkali metal carbonates such as caesium carbonate, sodium carbonate or potassium carbonate, or alkoxides such as potassium tert-butoxide or sodium tert-butoxide, preference being given to sodium hydroxide and lithium hydroxide.

The reaction in process [E] is carried out as described for process [B].

The compounds of the formula (X) are known, can be synthesized from the corresponding starting compounds by known processes or can be prepared analogously to the processes described in the Examples section.

The compounds of the formula (IX) are known or can be prepared by reacting

[F] compounds of the formula (V) with compounds of the formula

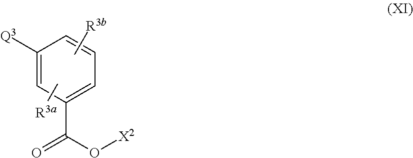

in which
R^{3a} and R^{3b} have the meaning given above,
X² represents methyl or ethyl and
Q³ represents —B(OH)₂, a boronic ester, preferably pinacol boronate, or —BF₃⁻K⁺,
under Suzuki coupling conditions,
or
[G] reacting compounds of the formula

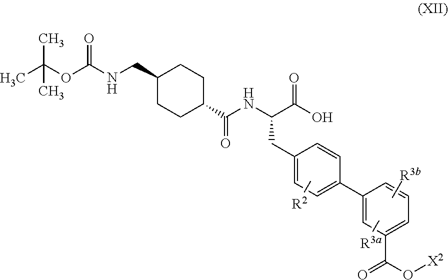

in which
R², R^{3a} and R^{3b} have the meaning given above and
X² represents methyl or ethyl,
with compounds of the formula (VIII) in the presence of a dehydrating reagent.

The reaction in process [F] is effected as described for process [B].

The compounds of the formula (XI) are known, can be synthesized from the corresponding starting compounds by known processes or can be prepared analogously to the processes described in the Examples section.

The reaction in process [G] is effected as described for process [A].

The compounds of the formula (V) are known or can be prepared by reacting compounds of the formula

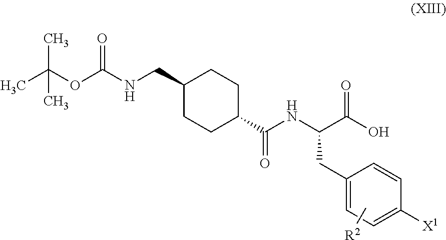

in which
R² has the meaning given above and
X¹ is bromine or iodine,
with compounds of the formula (VIII) in the presence of a dehydrating reagent.

The reaction is effected as described for process [A].

The compounds of the formula (XIII) are known, can be synthesized from the corresponding starting compounds by known processes or can be prepared analogously to the processes described in the Examples section.

The compounds of the formula (XII) are known or can be prepared by reacting compounds of the formula (XIII) with compounds of the formula (XI) under Suzuki coupling conditions.

The reaction is effected as described for process [B].

The compounds of the formula (VII) are known or can be prepared by reacting compounds of the formula (XIII) with compounds of the formula (VI) under Suzuki coupling conditions.

The reaction is effected as described for process [B].

The preparation of the starting compounds and of the compounds of the formula (I) can be illustrated by the synthesis scheme below.

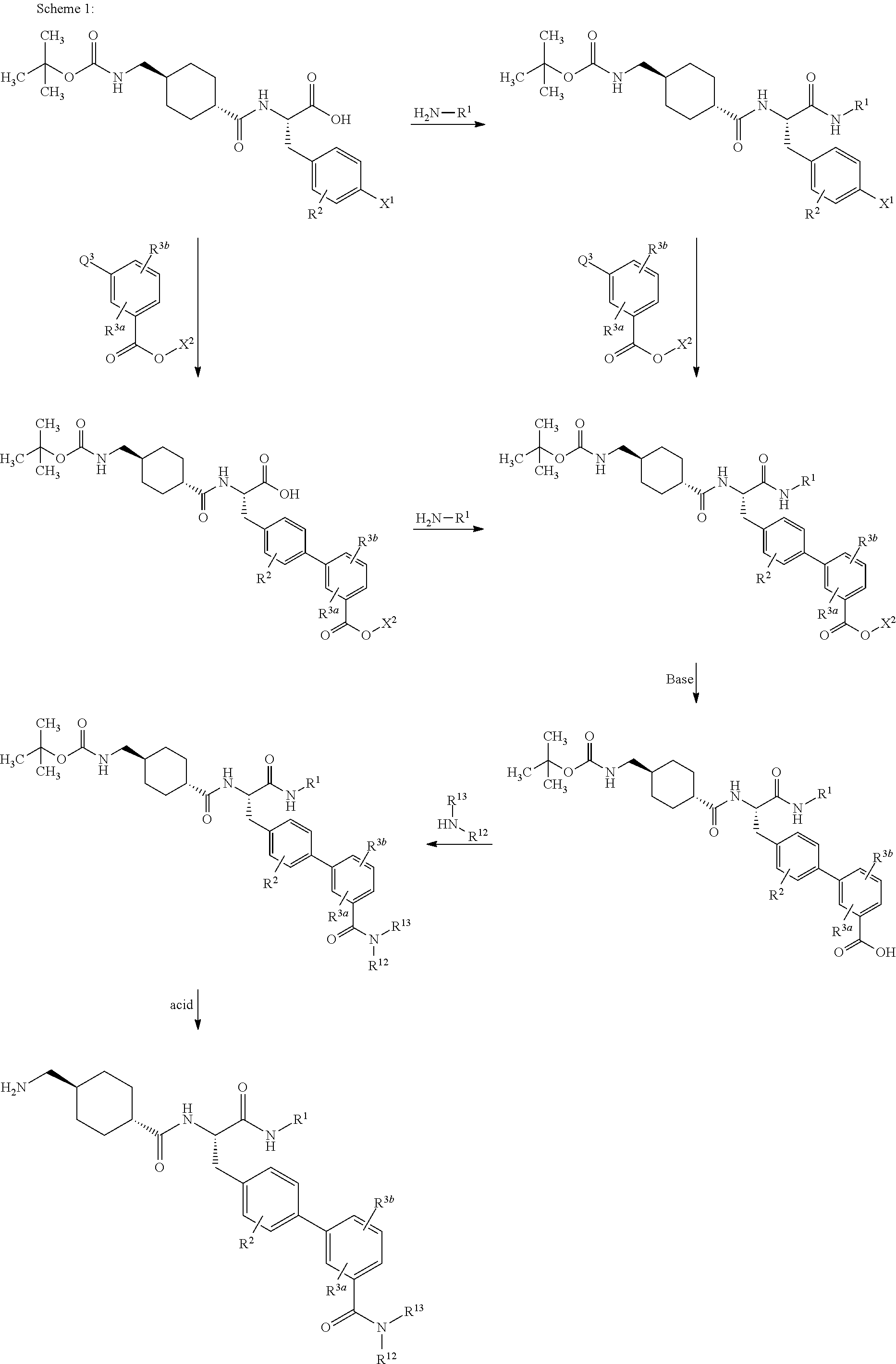

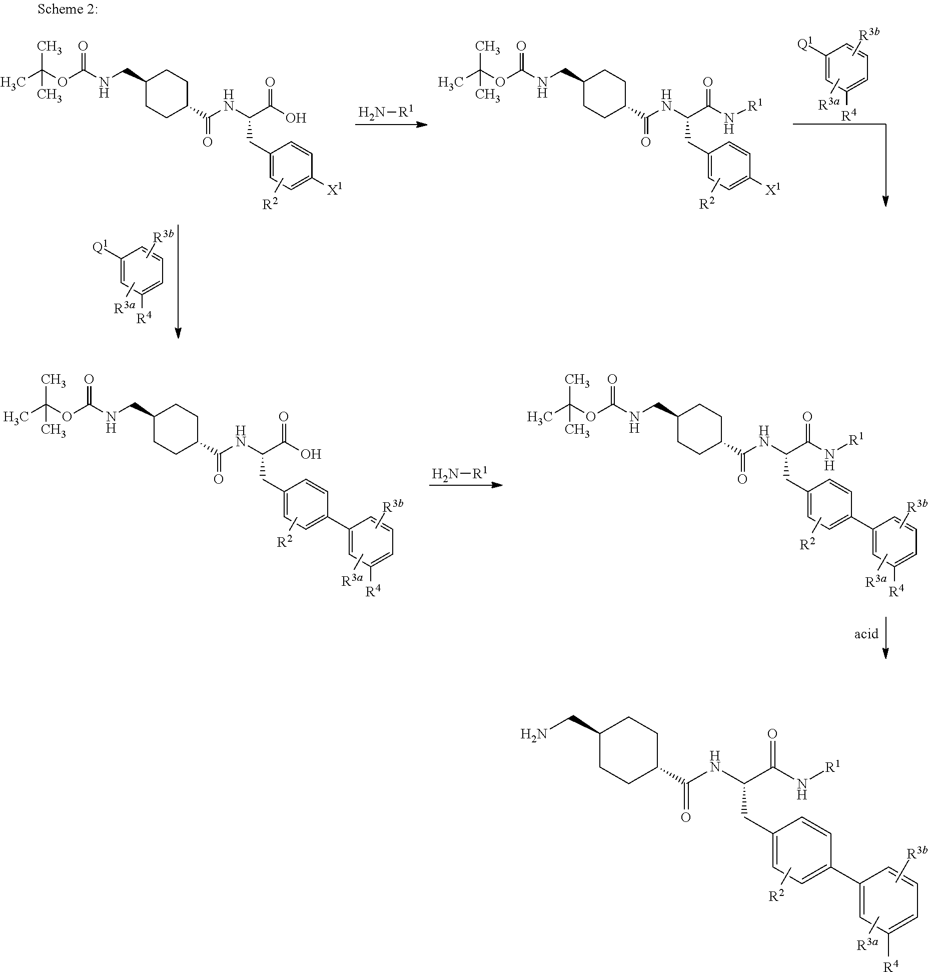

The compounds according to the invention have an unforeseeable useful pharmacological activity spectrum and good pharmacokinetic behaviour. They are compounds that influence the proteolytic activity of the serine proteases FXIa and kallikrein, and possibly plasmin. The inventive compounds inhibit the enzymatic cleavage of substrates that assume a major role in the activation of the blood coagulation cascade and platelet aggregation. If the inventive compounds inhibit plasmin activity, the result is inhibition of fibrinolysis.

They are therefore suitable for use as medicaments for treatment and/or prophylaxis of diseases in man and animals.

The present invention further provides for the use of the compounds according to the invention for the treatment and/or prophylaxis of disorders, in particular cardiovascular disorders, preferably thrombotic or thromboembolic disorders and/or thrombotic or thromboembolic complications.

“Thromboembolic disorders” in the sense of the present invention include in particular disorders such as acute coronary syndrome (ACS), ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, venous thromboses, especially in deep leg veins and renal veins, transitory ischaemic attacks and also thrombotic and thromboembolic stroke.

The inventive compounds are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alia, but also owing to surgical interventions, neoplastic disorders, burns or other injuries and may lead to severe organ damage through microthrombosis.

Thromboembolic complications are also encountered in microangiopathic haemolytic anaemias, extracorporeal circulatory systems, such as haemodialysis, and also prosthetic heart valves.

In addition, the inventive compounds are also used for influencing wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, inflammatory pulmonary disorders, glomerulonephritis and inflammatory intestinal disorders, for example Crohn's disease or ulcerative colitis or acute renal failure, and additionally likewise for the prophylaxis and/or treatment of dementia disorders, for example Alzheimer's disease. In addition, the inventive compounds can be used for inhibiting tumour growth and the formation of metastases, for microangiopathies, age-related macular degeneration, diabetic retinopathy, diabetic nephropathy and other microvascular disorders, and also for the prevention and treatment of thromboembolic complications, for example venous thromboembolisms, for tumour patients, especially those undergoing major surgery or chemo- or radiotherapy.

In addition, the inventive compounds are also suitable for the prophylaxis and/or treatment of pulmonary hypertension.

The term “pulmonary hypertension” includes certain forms of pulmonary hypertension, as determined, for example, by the World Health Organization (WHO). Examples include pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for the treatment and/or prophylaxis of pulmonary hypertension associated with sarcoidosis, histiocytosis X and lymphangiomatosis.

In addition, the inventive substances may also be useful for the treatment of pulmonary and hepatic fibroses.

In addition, the inventive compounds may also be suitable for the treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. This can be prevented by the inventive compounds. A secondary effect is the consumption of coagulation factors (for example factor X, prothrombin and fibrinogen) and platelets, which reduces the coagulability of the blood and may result in heavy bleeding.

In addition, the inventive compounds are also useful for the prophylaxis and/or treatment of hyperfibrinolysis. The prophylaxis and/or treatment may reduce or eliminate severe perioperative blood loss. Severe bleeding occurs in major operations, for example coronary artery bypass surgery, transplants or hysterectomy, and in the event of trauma, in the event of haemorrhagic shock or in the event of postpartum haemorrhage. In the aforementioned indications, there may be perioperative use of extracorporeal circulation systems or filter systems, for example heart and lung machines, haemofiltration, haemodialysis, extracorporeal membrane oxygenation or a ventricular support system, for example artificial heart. This additionally requires anticoagulation, for which the inventive compounds can also be used.

The inventive compounds are also suitable for anticoagulation during kidney replacement procedures, for example in the case of continuous veno-venous haemofiltration or intermittent haemodialysis.

In addition, the compounds according to the invention can also be used for preventing coagulation ex vivo, for example for preserving blood and plasma products, for cleaning/pretreating catheters and other medical auxiliaries and instruments, for coating synthetic surfaces of medical auxiliaries and instruments used in vivo or ex vivo or for biological samples which could contain factor XIa.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active compounds.

The present invention further provides a method for preventing the coagulation of blood in vitro, especially in banked blood or biological samples which could contain factor XIa, which is characterized in that an anticoagulatory amount of the inventive compound is added.

The present invention further provides medicaments comprising a compound according to the invention and one or more further active compounds, in particular for the treatment and/or prophylaxis of the disorders mentioned above. Preferred examples of active compounds suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors), for example tissue plasminogen activator (t-PA), streptokinase, reteplase and urokinase;
anticoagulatory substances (anticoagulants), for example heparin (UFH), low-molecular-weight heparins (LMW), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI), for example Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux;
platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), for example abciximab, eptifibatide, tirofiban, lamifiban, lefradafiban and fradafiban;
and also antiarrhythmics;
various antibiotics or antifungal medicaments, either as calculated therapy (prior to the presence of the microbial diagnosis) or as specific therapy;
vasopressors, for example norepinephrine, dopamine and vasopressin;
inotropic therapy, for example dobutamine;
corticosteroids, for example hydrocortisone and fludrocortisone;
recombinant human activated protein C such as, for example, Xigris;
blood products, for example erythrocyte concentrates, thrombocyte concentrates, erythropietin and fresh frozen plasma.

“Combinations” for the purpose of the invention mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Parenteral administration is preferred.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds of the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary, if appropriate, to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### A) EXAMPLES

### Abbreviations

bs/br. s. broad singlet (in NMR)
bd broad doublet (in NMR)
cat. catalytic
CI chemical ionization (in MS)
dd doublet of doublet (in NMR)
DMF dimethylformamide
DMSO dimethyl sulphoxide
dt doublet of triplet (in NMR)
EI electron impact ionization (in MS)
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure, high-performance liquid chromatography
LC-MS liquid chromatography-coupled mass spectrometry
m multiplet (in NMR)
M molar
min minute(s)
MS mass spectrometry
N normal
NMR nuclear magnetic resonance spectrometry
q quartet (in NMR)
quant. quantitative
quint quintet (in NMR)
RT room temperature
Rₜ retention time (in HPLC)
s singlet (in NMR)
TFA trifluoroacetic acid
THF tetrahydrofuran
UV ultraviolet spectrometry

### HPLC and LC/MS Methods:

### Method 1 (LC-MS):

Instrument: Waters ACQUITY SQD UPLC system; column: Waters Acquity UPLC HSS T3 1.8μ 50 mm×1 mm; mobile phase A: 1 l of water+0.25 ml of 99% strength formic acid, mobile phase B: 1 l of acetonitrile+0.25 ml of 99% strength formic acid; gradient 0.0 min 90% A→1.2 min 5% A→2.0 min 5% A; oven: 50° C.; flow rate: 0.40 ml/min; UV detection: 210-400 nm.

### Method 2 (LC-MS):

Instrument: Micromass Quattro Premier with Waters UPLC Acquity; column: Thermo Hypersil GOLD 1.9μ 50 mm×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

### Method 3 (LC-MS):

Instrument: Waters ACQUITY SQD UPLC system; column: Waters Acquity UPLC HSS T3 1.8μ 30 mm×2 mm; mobile phase A: 1 l of water+0.25 ml of 99% strength formic acid, mobile phase B: 1 l of acetonitrile+0.25 ml of 99% strength formic acid; gradient 0.0 min 90% A→1.2 min 5% A→2.0 min 5% A oven: 50° C.; flow rate: 0.60 ml/min; UV detection: 208-400 nm.

### Method 4 (LC-MS):

Instrument: Waters Acquity UPLC-MS SQD 3001; column: Acquity UPLC BEH C18 1.7μ 50 mm×2.1 mm; mobile phase A: water+0.1% formic acid, mobile phase B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.

### Method 5 (LC-MS):

Instrument: Waters Acquity UPLC-MS SQD 3001; column: Acquity UPLC BEH C18 1.7μ 50 mm×2.1 mm; mobile phase A: water+0.2% ammonia, mobile phase B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.

### Method 6 (HPLC):

System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile, gradient: A 95%/B 5%→A 55%/B 45%; flow rate: 150 ml/min; UV detection: 254 nm.

### Method 7 (HPLC):

System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile; gradient: A 90%/B 10%→A 50%/B 50%; flow rate: 150 ml/min; UV detection: 254 nm.

### Method 8 (HPLC):

System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile; gradient: A 85%/B 15%→A 45%/B 55%; flow rate: 150 ml/min; UV detection: 254 nm.

### Method 9 (HPLC):

System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile; gradient: A 80%/B 20%→A 40%/B 60%; flow rate: 150 ml/min; UV detection: 254 nm.

### Method 10 (HPLC):

Instrument: Waters autopurification system SQD; column: Waters XBridge C18 5μ 100 mm×30 mm; mobile phase A: water+0.1% formic acid (99%), mobile phase B: acetonitrile; gradient: 0-8.0 min 1-100% B, 8.0-10.0 min 100% B; flow rate 50.0 ml/min; temperature: RT; injection: 2500 μl; DAD scan: 210-400 nm.

### Method 11 (HPLC):

Instrument: Waters autopurification system SQD; column: Waters XBridge C18 5μ 100 mm×30 mm; mobile phase A: water+0.2% ammonia (32%), mobile phase B: acetonitrile; gradient: 0-8.0 min 1-100% B, 8.0-10.0 min 100% B; flow rate 50.0 ml/min; temperature: RT; injection: 2500 μl; DAD scan: 210-400 nm.

### Method 12 (LC-MS):

MS instrument: Waters (Micromass) QM; HPLC instrument: Agilent 1100 series; column: Agient ZORBAX Extend-C18 3.0 mm×50 mm 3.5 micron; mobile phase A: 1 l of water+0.01 mol of ammonium carbonate, mobile phase B: 1 l of acetonitrile; gradient 0.0 min 98% A→0.2 min 98% A→3.0 min 5% A→4.5 min 5% A; oven: 40° C.; flow rate: 1.75 ml/min; UV detection: 210 nm.

### Method 13 (LC-MS):

Instrument: Waters ACQUITY SQD UPLC system; column: Waters Acquity UPLC HSS T3 1.8μ 50 mm×1 mm; mobile phase A: 1 l of water+0.25 ml of 99% strength formic acid, mobile phase B: 1 l of acetonitrile+0.25 ml of 99% strength formic acid; gradient 0.0 min 95% A→6.0 min 5% A→7.5 min 5% A; oven: 50° C.; flow rate: 0.35 ml/min; UV detection: 210-400 nm.

### Method 14 (LC-MS):

MS instrument: Waters (Micromass) Quattro Micro; HPLC instrument: Agilent 1100 series; column: YMC-Triart C18 3μ 50 mm×3 mm; mobile phase A: 1 l of water+0.01 mol of ammonium carbonate, mobile phase B: 1 l of acetonitrile; gradient: 0.0 min 10 0% A→2.75 min 5% A→4.5 min 5% A; oven: 40° C.; flow rate: 1.25 ml/min; UV detection: 210 nm.

### Method 15 (HPLC):

System: Labomatic HD-3000 HPLC gradient pump, Labomatic Labocol Vario-2000 fraction collector; column: Chromatorex C-18 125 mm×30 mm; mobile phase A: 0.1% ammonia in water, mobile phase B: acetonitrile, gradient: A 90%/B 10%→A 50%/B 50%; flow rate: 150 ml/min; UV detection: 254 nm.

### Microwave:

The microwave reactor used was an instrument of the Biotage™ Initiator type.

When compounds according to the invention are purified by preparative HPLC by the above-described methods in which the eluents contain additives, for example trifluoroacetic acid, formic acid or ammonia, the compounds according to the invention may be obtained in salt form, for example as trifluoroacetate, formate or ammonium salt, if the compounds according to the invention contain a sufficiently basic or acidic functionality Such a salt can be converted to the corresponding free base or acid by various methods known to the person skilled in the art. Weaker salts can be converted to the corresponding chlorides by addition of a little hydrochloride.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

If the starting compounds and examples contain an L-phenylalanine derivative as the central unit, the corresponding stereocentre is described as the (S) configuration. In the absence of further information, there was no check in individual cases as to whether partial epimerization of the stereocentre took place in the coupling of the L-phenylalanine intermediate with the amine H₂N—R¹. Thus, a mixture of the inventive compounds of (S) enantiomer and (R) enantiomer may be present. The main component is the (S) enantiomer depicted in each case.

### Starting Materials

### Example 1A

### Methyl N-[(trans-4-{[(tert-butoxycarbonyl)amino]methyl}cyclohexyl)carbonyl]-4-iodo-L-phenylalaninate

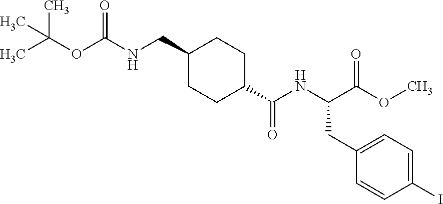

Methyl 4-iodo-L-phenylalaninate hydrochloride (5.7 g, 16.7 mmol), trans-4-{[(tert-butoxycarbonyl)amino]methyl}cyclohexanecarboxylic acid (4.4 g, 16.7 mmol) and N,N-diisopropylethylamine (11.7 ml, 67 mmol) were suspended in 90 ml of ethyl acetate. The solution was cooled to 0° C. Subsequently, 2,4,6-tripropyl-1,3,5,2,4,6-trioxatriphosphinane 2,4,6-trioxide (50% in ethyl acetate, 26.6 g, 42 mmol) was added dropwise, and the mixture was stirred at 0° C. for 30 minutes and at RT overnight. The mixture was quenched with water and extracted three times with ethyl acetate. The combined organic phases were washed once with saturated aqueous ammonium chloride solution and once with saturated aqueous sodium chloride solution, dried over magnesium sulphate, filtered and concentrated to dryness. The residue was recrystallized from acetonitrile. This gave 5.6 g (73% of theory) of the title compound.

¹H-NMR (300 MHz, DMSO-d₆): δ ppm 0.68-0.86 (m, 2H), 1.02-1.27 (m, 3H), 1.33 (s, 9H), 1.45-1.55 (m, 1H), 1.62 (m, 3H), 1.92-2.04 (m, 1H), 2.70 (t, 2H), 2.79 (dd, 1H), 2.94 (dd, 1H), 3.56 (s, 3H), 4.27-4.44 (m, 1H), 6.69-6.79 (m, 1H), 6.98 (d, 2H), 7.59 (d, 2H), 8.10 (d, 1H).

LC-MS (Method 4): Rₜ=1.32 min; MS (ESIpos): m/z=545.2 [M+H]⁺.

### Example 2A

### N-[(trans-4-{[(tert-Butoxycarbonyl)amino]methyl}cyclohexyl)carbonyl]-4-iodo-L-phenylalanine

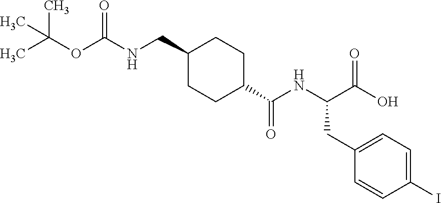

Methyl 4-iodo-N-[(trans-4-{[(tert-butoxycarbonyl)amino]methyl}cyclohexyl)carbonyl]-L-phenylalaninate (3.8 g, 7.0 mmol) was dissolved in 55 ml of tetrahydrofuran, the mixture was cooled to 0° C. and 5.3 ml of 2N aqueous sodium hydroxide solution were added. The mixture was allowed to come to RT and stirred at RT overnight. Subsequently, the tetrahydrofuran was drawn off and the aqueous phase was washed twice with tert-butyl methyl ether. The aqueous phase was then adjusted to pH 3 with 1N hydrochloric acid and the precipitated solid was filtered off with suction. The aqueous phase was extracted three times with dichloromethane and the organic phase was concentrated. The residue from the organic phase was combined with the solid and dried under high vacuum. This gave 3.8 g (100% of theory) of the title compound.

¹H-NMR (400 MHz, DMSO-d₆): δ ppm 0.72-0.85 (m, 2H), 1.08-1.27 (m, 3H), 1.33 (s, 9H), 1.63 (m, 4H), 1.87-1.96 (m, 1H), 2.70 (t, 2H), 2.83 (dd, 1H), 2.95 (dd, 1H), 3.83 (m, 1H), 6.69-6.75 (m, 1H), 6.84 (d, 2H), 6.93 (d, 1H), 7.47 (d, 2H).

LC-MS (Method 4): Rₜ=1.20 min; MS (ESIpos): m/z=531.1 [M+H]⁺.

### Example 3A

### N-alpha-[(trans-4-{[(tert-Butoxycarbonyl)amino]methyl}cyclohexyl)carbonyl]-N-[4-(5-chloro-4H-1,2,4-triazol-3-yl)phenyl]-4-iodo-L-phenylalaninamide

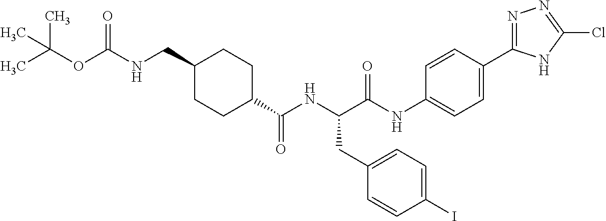

N-[(trans-4-{[(tert-Butoxycarbonyl)amino]methyl}cyclohexyl)carbonyl]-4-iodo-L-phenylalanine (2.4 g, 4.5 mmol), 4-(5-chloro-4H-1,2,4-triazol-3-yl)aniline (70%, 1.4 g, 5.0 mmol) and triethylamine (1.6 ml, 11 mmol) were suspended in 44 ml of ethyl acetate, and 2,4,6-tripropyl-1,3,5,2,4,6-trioxatriphosphinane 2,4,6-trioxide (50% in ethyl acetate, 5.3 ml, 9.0 mmol) was added. The mixture was then heated under reflux for 2 h and stirred at RT for a further 24 h. Water was added to the reaction mixture and the precipitated solid was filtered off with suction, washed with a little ethyl acetate and water and dried under high vacuum. This gave 2.5 g (78% of theory) of the title compound.

¹H-NMR (400 MHz, DMSO-d₆): δ ppm 0.78 (m, 2H), 1.05-1.27 (m, 4H), 1.33 (s, 9H), 1.48-1.55 (m, 1H), 1.64 (m, 3H), 2.00-2.10 (m, 1H), 2.71 (t, 2H), 2.79 (dd, 1H), 2.95 (dd, 1H), 4.55-4.65 (m, 1H), 6.68-6.77 (m, 1H), 7.07 (d, 2H), 7.60 (d, 2H), 7.70 (d, 2H), 7.86 (d, 2H), 8.06 (d, 1H), 10.33 (s, 1H).

LC-MS (Method 4): Rₜ=1.31 min; MS (ESIpos): m/z=707.3 [M+H]⁺.

### Example 4A

### Methyl 4-bromo-N-[(trans-4-{[(tert-butoxycarbonyl)amino]methyl}cyclohexyl)carbonyl]-L-phenylalaninate

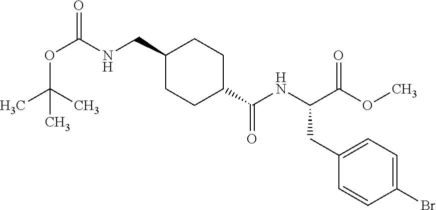

A solution of methyl 4-bromo-L-phenylalaninate (250 g, 874 mmol) and trans-4-{[(tert-butoxycarbonyl)amino]methyl}cyclohexanecarboxylic acid (225 g, 874 mmol) in ethyl acetate (5012 ml) was admixed with N,N-diisopropylethylamine (381 ml, 2186 mmol). The suspension was admixed dropwise with a 2,4,6-tripropyl-1,3,5,2,4,6-trioxatriphosphinane 2,4,6-trioxide solution (50% in dimethylformamide, 766 ml, 1312 mmol) and then the mixture was stirred at RT for 3 h.

The reaction mixture was then stirred into water and extracted three times with ethyl acetate. The organic phase was washed with saturated aqueous sodium hydrogencarbonate solution, saturated aqueous ammonium chloride solution, and saturated aqueous sodium chloride solution. The solution was dried over sodium sulphate and the solvent was removed. This gave 420 g (97% of theory) of the title compound.

¹H-NMR (400 MHz, DMSO-d₆): δ=0.68-0.92 (m, 2H), 1.04-1.32 (m, 4H), 1.37 (s, 9H), 1.48-1.73 (m, 4H), 2.03 (m, 1H), 2.74 (m, 2H), 2.78-2.90 (m, 1H), 2.94-3.05 (m, 1H), 4.36-4.50 (m, 1H), 6.72-6.85 (m, 1H), 7.17 (d, 2H), 7.46 (d, 2H), 8.15 (d, 1H).

LC-MS (Method 1): Rₜ=1.14 min; MS (ESIpos): m/z=497 [M+H]⁺.

### Example 5A

### 4-Bromo-N-[(trans-4-{[(tert-butoxycarbonyl)amino]methyl}cyclohexyl)carbonyl]-L-phenylalanine

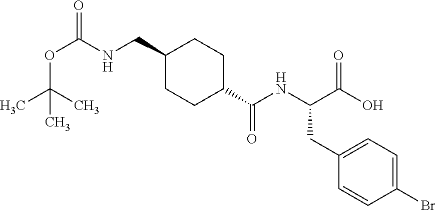

A solution of methyl 4-bromo-N-[(trans-4-{[(tert-butoxycarbonyl)amino]methyl}-cyclohexyl)carbonyl]-L-phenylalaninate in tetrahydrofuran (3000 ml) was admixed with a solution of lithium hydroxide (72 g, 3015 mmol) in water (600 ml). The suspension was stirred at RT for 16 h. The reaction mixture was acidified with 1N hydrochloric acid solution and admixed with ethyl acetate. The organic phase was washed with saturated aqueous sodium chloride solution and dried over sodium sulphate, and the solvent was removed. This gave 284 g (97% of theory) of the title compound.

¹H-NMR (400 MHz, DMSO-d₆): δ=0.71-0.90 (m, 2H), 1.22 (d, 4H), 1.37 (s, 9H), 1.45-1.73 (m, 5H), 2.03 (m, 1H), 2.67-2.88 (m, 3H), 2.95-3.09 (m, 1H), 4.38 (m, 1H), 6.77 (s, 1H), 7.17 (d, 2H), 7.46 (d, 2H), 7.99 (d, 1H), 12.65 (br. s, 1H).

LC-MS (Method 1): Rₜ=1.03 min; MS (ESIneg): m/z=481 [M−H]⁻.

### Example 6A

### 4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide

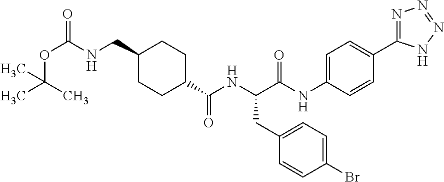

A solution of 4-bromo-N-[(trans-4-{[(tert-butoxycarbonyl)amino]methyl}cyclohexyl)carbonyl]-L-phenylalanine (11 g, 22 mmol) and 4-(1H-tetrazol-5-yl)aniline (4 g, 24 mmol) in dimethylformamide (161 ml) was admixed with N,N-diisopropylethylamine (9.6 ml, 55 mmol). The suspension was admixed dropwise at 0° C. with a 2,4,6-tripropyl-1,3,5,2,4,6-trioxatriphosphinane 2,4,6-trioxide solution (50% in dimethylformamide, 16.9 g, 27 mmol) and then the mixture was stirred at RT for 16 h. The reaction mixture was stirred into ethyl acetate (13 000 ml) and extracted three times with water (1570 ml each time). The organic phase was dried with sodium sulphate and the solvent was removed. The crude product was stirred with acetonitrile and filtered off with suction. This gave 11.4 g (78% of theory) of the title compound.

¹H-NMR (400 MHz, DMSO-d₆): δ=0.67-0.90 (m, 2H), 1.24 (m, 4H), 1.37 (s, 9H), 1.51-1.74 (m, 4H), 2.02-2.17 (m, 1H), 2.71-2.79 (m, 2H), 2.79-2.89 (m, 1H), 2.99-3.06 (m, 1H), 3.06-3.16 (m, 1H), 3.51-3.67 (m, 1H), 4.55-4.74 (m, 1H), 6.01-6.02 (m, 1H), 6.69-6.84 (m, 1H), 7.21-7.32 (m, 2H), 7.43-7.55 (m, 2H), 7.64-7.76 (m, 2H), 7.88-7.99 (m, 2H), 8.03-8.14 (m, 1H), 10.25 (s, 1H).

LC-MS (Method 1): Rₜ=1.07 min; MS (ESIneg): m/z=624 [M−H]⁻.

### Example 7A

### 4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide

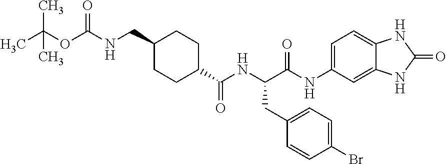

A solution of 4-bromo-N-[(trans-4-{[(tert-butoxycarbonyl)amino]methyl}cyclohexyl)carbonyl]-L-phenylalanine (1500 mg, 3 mmol) and 5-amino-1,3-dihydro-2H-benzimidazol-2-one (555 mg, 24 mmol) in ethyl acetate (21 ml) was admixed with N,N-diisopropylethylamine (1.4 ml, 7.8 mmol). A 2,4,6-tripropyl-1,3,5,2,4,6-trioxatriphosphinane 2,4,6-trioxide solution (50% in dimethylformamide, 2.2 ml, 3.7 mmol) and, until dissolution, dimethylformamide were added to the suspension, and the mixture was then stirred at RT for 16 h. The reaction mixture was stirred into ethyl acetate, and washed twice with water and once with saturated aqueous sodium chloride solution. The organic phase was dried with sodium sulphate and the solvent was removed. The crude product was stirred with acetonitrile and filtered off with suction. The residue was separated twice by preparative HPLC (mobile phase: gradient of acetonitrile/water with 0.1% trifluoroacetic acid). The crude product was stirred with methanol and filtered off with suction. This gave 202 mg (11% of theory) of the title compound.

¹H-NMR (400 MHz, DMSO-d₆): δ=0.69-0.89 (m, 2H), 1.04-1.29 (m, 3H), 1.37 (s, 9H), 1.67 (m, 4H), 2.04-2.17 (m, 1H), 2.75 (m, 3H), 2.94-3.07 (m, 1H), 4.54-4.75 (m, 1H), 6.68-6.83 (m, 1H), 6.96 (dd, 1H), 7.25 (d, 2H), 7.39-7.56 (m, 3H), 7.84 (s, 1H), 8.09 (d, 1H), 10.20 (s, 1H), 11.08 (br. s, 1H).

LC-MS (Method 1): Rₜ=1.00 min; MS (ESIpos): m/z=614 [M+H]⁺.

### Example 8A

### 4-Bromo-N-alpha-[(trans-4-{[(tert-butoxycarbonyl)amino]methyl}cyclohexyl)carbonyl]-N-1H-indazol-6-yl-L-phenylalaninamide

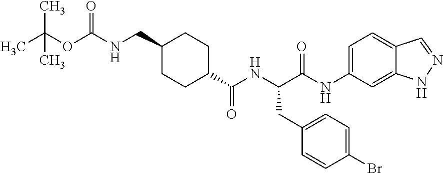

N,N-Diisopropylethylamine (1.8 ml, 10 mmol) was added to a solution of 4-bromo-N-[(trans-4-{[(tert-butoxycarbonyl)amino]methyl}cyclohexyl)carbonyl]-L-phenylalanine (2000 mg, 4 mmol) and 6-aminoindazole (606 mg, 5 mmol) in dimethylformamide (30 ml). The suspension was admixed with a 2,4,6-tripropyl-1,3,5,2,4,6-trioxatriphosphinane 2,4,6-trioxide solution (50% in dimethylformamide, 3.2 mg, 5 mmol) and with dimethylformamide until dissolution, and then the mixture was stirred at RT for 16 h. The reaction mixture was stirred into ethyl acetate (2500 ml), and washed three times with water (300 ml) and once with saturated aqueous sodium chloride solution. The organic phase was dried with sodium sulphate and the solvent was removed. The crude product was stirred with acetonitrile and filtered off with suction. This gave 1400 mg (54% of theory) of the title compound.

¹H-NMR (400 MHz, DMSO-d₆): δ=0.68-0.98 (m, 2H), 1.05-1.31 (m, 4H), 1.39 (s, 9H), 1.46-1.76 (m, 4H), 1.98-2.15 (m, 1H), 2.65-3.07 (m, 4H), 4.56-4.71 (m, 1H), 6.71-6.83 (m, 1H), 7.25 (d, 2H), 7.47 (d, 2H), 7.72-7.84 (m, 4H), 8.10-8.20 (m, 1H), 10.45 (s, 1H), 12.86 (br. s, 1H).

LC-MS (Method 1): Rₜ=1.09 min; MS (ESIpos): m/z=598 [M+H]⁺.

### Example 9A

### Ethyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-fluorobiphenyl-3-carboxylate

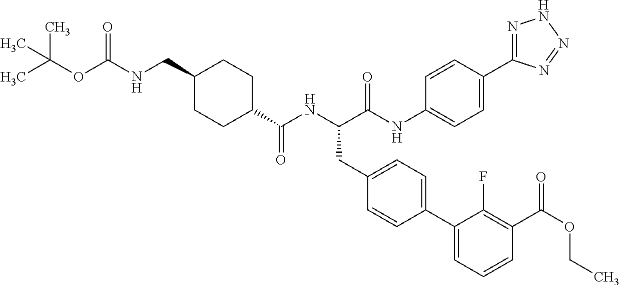

1000 mg (1.60 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 184 mg (0.16 mmol) of tetrakis(triphenylphosphine)palladium(0) and 677 mg (3.19 mmol) of [3-(ethoxycarbonyl)-2-fluorophenyl]boronic acid were taken up in 12 ml of 1,2-dimethoxyethane and 5 ml of ethanol After the addition of 3 ml of 2N aqueous sodium carbonate solution, the mixture was stirred under reflux for 4 h and at RT for 16 h. The reaction mixture was filtered through kieselguhr, and the solvent was removed. The residue of the filtrate was separated by column chromatography on silica gel (mobile phase: ethyl acetate→ethyl acetate/methanol 8:2). This gave 977 mg (82% of theory) of the title compound.

¹H-NMR (400 MHz, DMSO-d₆): δ=0.74-0.92 (m, 2H), 1.09-1.42 (m, 16H), 1.50-1.77 (m, 4H), 2.05-2.18 (m, 1H), 2.70-2.78 (m, 2H), 2.88-2.99 (m, 2H), 3.08-3.18 (m, 1H), 4.28-4.39 (m, 2H), 4.67-4.76 (m, 1H), 6.72-6.84 (m, 1H), 7.34-7.51 (m, 4H), 7.52-7.66 (m, 1H), 7.68-7.74 (m, 1H), 7.76-7.87 (m, 3H), 7.94-8.02 (m, 2H), 8.19 (d, 1H), 10.43 (s, 1H).

LC-MS (Method 1): Rₜ=1.15 min; MS (ESIneg): m/z=712 [M−H]⁻.

### Example 10A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-fluorobiphenyl-3-carboxylic acid

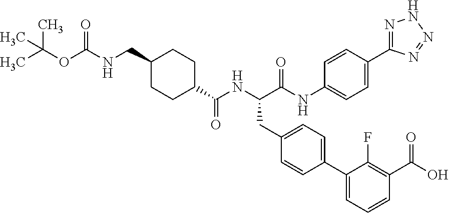

915 mg (1.28 mmol) of ethyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-fluorobiphenyl-3-carboxylate were dissolved in 22.5 ml of tetrahydrofuran and 7.5 ml of water, 538 mg (12.82 mmol) of lithium hydroxide monohydrate were added and the mixture was stirred at RT for 16 h. 50 ml of water and 1N hydrochloric acid to pH 4 were added to the reaction mixture. The mixture was extracted three times with 100 ml of dichloromethane and the organic phase was dried over sodium sulphate, filtered and concentrated. The residue was stirred with 10 ml of acetonitrile in an ultrasonic bath, filtered and concentrated to dryness. This gave 604 mg (66% of theory) of the title compound.

¹H-NMR (400 MHz, DMSO-d₆): δ=0.73-0.93 (m, 2H), 1.09-1.32 (m, 3H), 1.36 (s, 9H), 1.50-1.76 (m, 4H), 2.06-2.18 (m, 1H), 2.69-2.80 (m, 2H), 2.90-3.00 (m, 1H), 3.07-3.17 (m, 1H), 4.67-4.79 (m, 1H), 6.74-6.83 (m, 1H), 7.36 (t, 1H), 7.40-7.51 (m, 3H), 7.52-7.72 (m, 2H), 7.78-7.87 (m, 3H), 8.00 (d, 2H), 8.16-8.25 (m, 1H), 10.48 (s, 1H), 13.3 (br. s, 1H), 16.7 (br. s, 1H).

LC-MS (Method 1): Rₜ=0.98 min; MS (ESIneg): m/z=684 [M−H]⁻.

### Example 11A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-methylbiphenyl-3-carboxylate

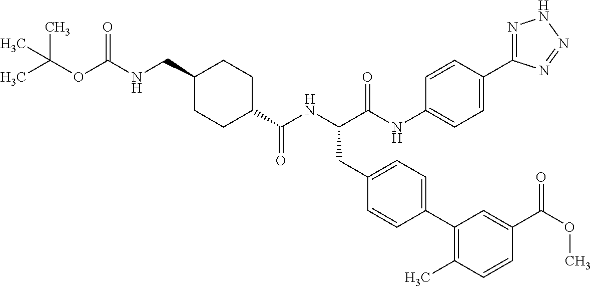

1000 mg (1.60 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 184 mg (0.16 mmol) of tetrakis(triphenylphosphine)palladium(0) and 881 mg (3.19 mmol) of methyl 4-methyl-3-(4,4,5,5-tetramethyl-1,3,2-dioxaborolan-2-yl)benzoate were taken up in 12 ml of 1,2-dimethoxyethane and 5 ml of ethanol. After addition of 3 ml of 2N aqueous sodium carbonate solution, the reaction mixture was stirred at reflux for 4 h and at RT for 16 h, the salts were then filtered off over kieselguhr and the filtrate was separated by column chromatography on silica gel (mobile phase: ethyl acetate→ethyl acetate/methanol 8:2). This gave 1024 mg (88% of theory) of the title compound.

¹H-NMR (400 MHz, DMSO-d₆): δ=0.73-0.92 (m, 2H), 1.09-1.31 (m, 3H), 1.36 (s, 9H), 1.48-1.57 (m, 1H), 1.59-1.73 (m, 3H), 2.06-2.16 (m, 1H), 2.24 (s, 3H), 2.69-2.79 (m, 2H), 2.89-3.00 (m, 1H), 3.08-3.18 (m, 1H), 3.83 (s, 3H), 4.70-4.81 (m, 1H), 6.74-6.82 (m, 1H), 7.28 (d, 2H), 7.35-7.48 (m, 3H), 7.68-7.73 (m, 1H), 7.74-7.81 (m, 2H), 7.81-7.86 (m, 1H), 7.93-8.02 (m, 2H), 8.18 (d, 1H), 10.39 (s, 1H), 16.7 (br. s, 1H).

LC-MS (Method 1): Rₜ=1.14 min; MS (ESIneg): m/z=694 [M−H]⁻.

### Example 12A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-6-methylbiphenyl-3-carboxylic acid

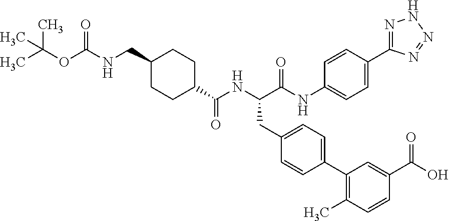

965 mg (1.39 mmol) of methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-methylbiphenyl-3-carboxylate were dissolved in 22.5 ml of tetrahydrofuran and 7.5 ml of water, 582 mg (13.87 mmol) of lithium hydroxide monohydrate were added and the mixture was stirred at RT for 16 h. Two thirds of the solvent were removed from the reaction mixture. 20 ml of water and 1N hydrochloric acid to pH 4 were added to the remaining suspension. The precipitate was filtered off and washed with a little water and the crude product was dried under high vacuum. This gave 650 mg (63% of theory, 92% pure) of the title compound.

¹H-NMR (400 MHz, DMSO-d₆): δ=0.72-0.92 (m, 2H), 1.04-1.30 (m, 3H), 1.36 (s, 9H), 1.47-1.57 (m, 1H), 1.59-1.76 (m, 3H), 2.04-2.17 (m, 1H), 2.24 (s, 3H), 2.69-2.79 (m, 2H), 2.87-3.00 (m, 1H), 3.06-3.18 (m, 1H), 4.68-4.82 (m, 1H), 6.71-6.84 (m, 1H), 7.28 (d, J=8.07 Hz, 2H), 7.34-7.45 (m, 3H), 7.50-7.66 (m, 1H), 7.68-7.78 (m, 3H), 7.79-7.85 (m, 1H), 7.96 (d, J=8.56 Hz, 2H), 8.13-8.21 (m, 1H), 10.33 (s, 1H).

LC-MS (Method 1): Rₜ=1.10 min; MS (ESIneg): m/z=680 [M−H]⁻.

### Example 13A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-3-carboxylate

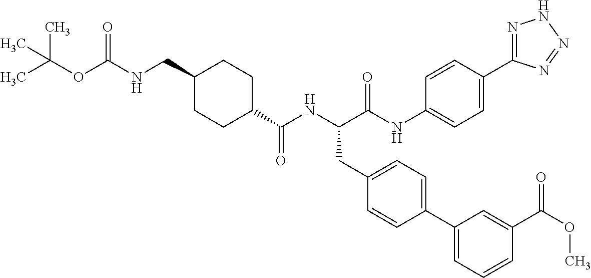

1000 mg (1.60 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 184 mg (0.16 mmol) of tetrakis(triphenylphosphine)palladium(0) and 574 mg (3.19 mmol) of [3-(methoxycarbonyl)phenyl]boronic acid were taken up in 12 ml of 1,2-dimethoxyethane and 5 ml of ethanol. After addition of 3 ml of 2N aqueous sodium carbonate solution, the reaction mixture was stirred at reflux for a total of 8 h and at RT for 16 h, the salts were then filtered off over kieselguhr and the filtrate was separated by column chromatography on silica gel (mobile phase: ethyl acetate→ethyl acetate/methanol 8:2). The product fraction was concentrated and the solid was briefly boiled with 8 ml of acetonitrile and agitated in an ultrasonic bath for 1 min. The solid was filtered off, washed with 3 ml of acetonitrile and dried under high vacuum. This gave 466 mg (40% of theory, 94% pure) of the title compound.

¹H-NMR (400 MHz, DMSO-d₆): δ=0.73-0.92 (m, 2H), 1.08-1.30 (m, 3H), 1.36 (s, 9H), 1.52-1.76 (m, 4H), 2.06-2.17 (m, 1H), 2.70-2.78 (m, 2H), 2.88-2.99 (m, 1H), 3.07-3.15 (m, 1H), 3.88 (s, 3H), 4.67-4.77 (m, 1H), 6.74-6.82 (m, 1H), 7.44 (d, 2H), 7.57-7.69 (m, 3H), 7.80 (d, 2H), 7.90-8.02 (m, 4H), 8.13-8.22 (m, 2H), 10.44 (s, 1H).

LC-MS (Method 1): Rₜ=1.11 min; MS (ESIneg): m/z=680 [M−H]⁻.

### Example 14A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]biphenyl-3-carboxylic acid

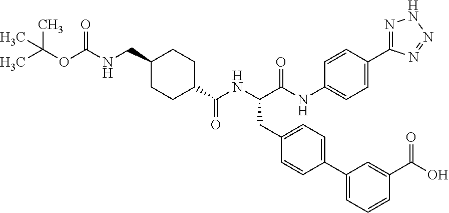

465 mg (0.53 mmol, 78% pure) of methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-3-carboxylate were dissolved in 15 ml of tetrahydrofuran and 5 ml of water, 223 mg (5.31 mmol) of lithium hydroxide monohydrate were added and the mixture was stirred at RT for 16 h. 150 ml of water and 1N hydrochloric acid to pH 4 were added to the reaction mixture. The precipitate was filtered off and washed with a little water and the crude product was dried under high vacuum. This gave 418 mg (78% of theory, 66% pure) of the title compound.

¹H-NMR (400 MHz, DMSO-d₆): δ=0.72-0.92 (m, 2H), 1.09-1.31 (m, 3H), 1.36 (s, 9H), 1.53-1.77 (m, 4H), 2.07-2.17 (m, 1H), 2.69-2.79 (m, 2H), 2.88-2.99 (m, 1H), 3.05-3.16 (m, 1H), 4.67-4.78 (m, 1H), 6.72-6.82 (m, 1H), 7.43 (d, 2H), 7.55-7.68 (m, 3H), 7.82 (d, 2H), 7.87-7.94 (m, 2H), 8.00 (d, 2H), 8.13-8.23 (m, 2H), 10.49 (s, 1H), 13.1 (br. s, 1H), 16.8 (br. s, 1H).

LC-MS (Method 1): Rₜ=0.99 min; MS (ESIneg): m/z=666 [M−H]⁻.

### Example 15A

### tert-Butyl [(trans-4-{[(2S)-3-[5′-(dimethylsulphamoyl)-2′-methylbiphenyl-4-yl]-1-oxo-1-{[4-(1H-tetrazol-5-yl)phenyl]amino}propan-2-yl]carbamoyl}cyclohexyl)methyl]carbamate

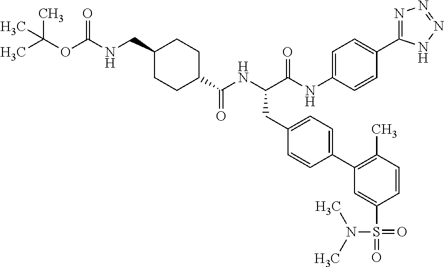

4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [5-(dimethylsulphamoyl)-2-methylphenyl]boronic acid (87 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 57 mg (32% of theory) of the title compound.

LC-MS (Method 5): Rₜ=0.89 min; MS (ESIpos): m/z=745.5 [M+H]⁺.

### Example 16A

### tert-Butyl [(trans-4-{[(2S)-3-[2′-methyl-5′-(morpholin-4-ylsulphonyl)biphenyl-4-yl]-1-oxo-1-{[4-(1H-tetrazol-5-yl)phenyl]amino}propan-2-yl]carbamoyl}cyclohexyl)methyl]carbamate

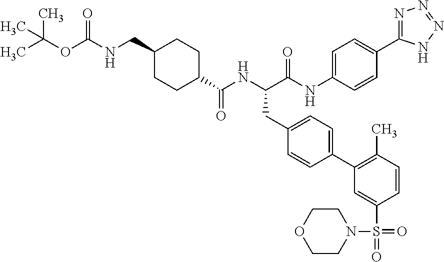

4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [2-methyl-5-(morpholin-4-ylsulphonyl)phenyl]boronic acid (102 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 100 mg (53% of theory) of the title compound.

LC-MS (Method 5): Rₜ=0.88 min; MS (ESIpos): m/z=787.5 [M+H]⁺.

### Example 17A

### tert-Butyl {[trans-4-({(2S)-1-{[4-(5-chloro-4H-1,2,4-triazol-3-yl)phenyl]amino}-3-[5′-(dimethylsulphamoyl)-2′-methylbiphenyl-4-yl]-1-oxopropan-2-yl}carbamoyl)cyclohexyl]methyl}carbamate

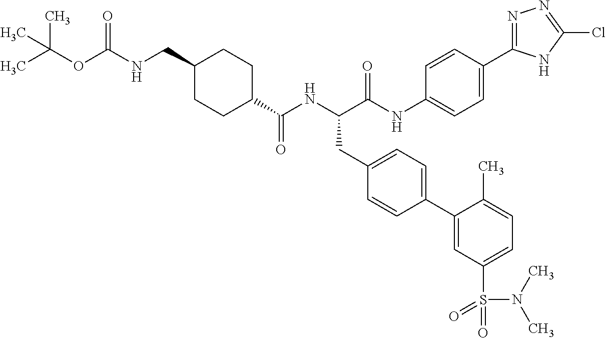

N-alpha-[(trans-4-{[(tert-Butoxycarbonyl)amino]methyl}cyclohexyl)carbonyl]-N-[4-(5-chloro-4H-1,2,4-triazol-3-yl)phenyl]-4-iodo-L-phenylalaninamide (150 mg, 0.21 mmol) and [5-(dimethylsulphamoyl)-2-methylphenyl]boronic acid (79 mg, 0.32 mmol) were dissolved in dimethyl sulphoxide (2 ml), and tetrakis(triphenylphosphine)palladium(0) (24 mg, 21 μmol), sodium carbonate (67 mg, 0.64 mmol) and water (0.32 ml, 18 mmol) were added. The reaction mixture was stirred at 110° C. in a microwave (Biotage Initiator) for 90 min, cooled, filtered and purified by chromatography via HPLC (Method 11). This gave 34 mg (21% of theory) of the title compound.

LC-MS (Method 5): Rₜ=0.88 min; MS (ESIpos): m/z=778.5 [M+H]⁺.

### Example 18A

### tert-Butyl [(trans-4-{[(2S)-1-oxo-3-(3′-sulphamoylbiphenyl-4-yl)-1-{[4-(1H-tetrazol-5-yl)phenyl]-amino}propan-2-yl]carbamoyl}cyclohexyl)methyl]carbamate

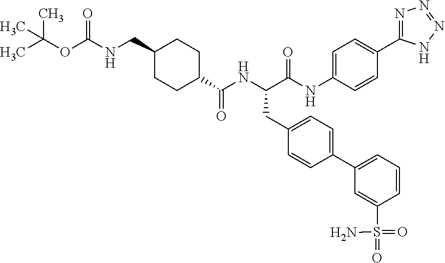

4-Bromo-N-alpha-[(trans-4-{[(tert-butoxycarbonyl)amino]methyl}cyclohexyl) carbonyl]-N-[4-(1H-tetrazol-5-yl)phenyl]-L-phenylalaninamide (150 mg, 0.24 mmol) and (3-sulphamoylphenyl)boronic acid (72 mg, 0.36 mmol) were dissolved in dimethyl sulphoxide (2 ml), and tetrakis(triphenylphosphine)palladium(0) (28 mg, 24 μmol), sodium carbonate (76 mg, 0.72 mmol) and water (0.36 ml, 20 mmol) were added. The reaction mixture was stirred at 110° C. in a microwave (Biotage Initiator) for 120 min, cooled, filtered and purified by chromatography via HPLC (Method 11). This gave 91 mg (54% of theory) of the title compound.

LC-MS (Method 5): Rₜ=0.76 min; MS (ESIpos): m/z=703.4 [M+H]⁺.

### Example 19A

### tert-Butyl [(trans-4-{[(2S)-3-{3′-[(4-methylpiperazin-1-yl)carbonyl]biphenyl-4-yl}-1-oxo-1-{[4-(1H-tetrazol-5-yl)phenyl]amino}propan-2-yl]carbamoyl}cyclohexyl)methyl]carbamate formate

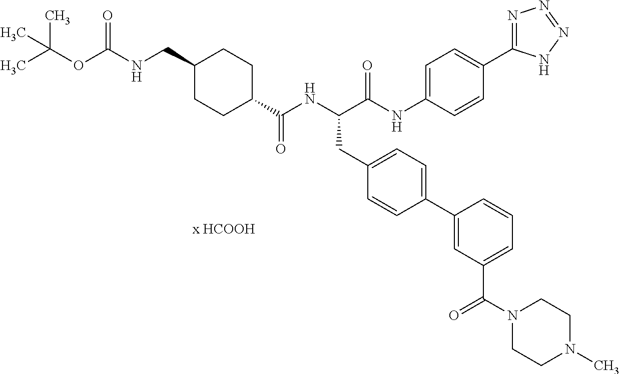 [SMILES: CN1CCN(C(=O)c2cccc(-c3ccc(C[C@H](NC(=O)[C@H]4CC[C@H](CNC(=O)OC(C)(C)C)CC4)C(=O)Nc4ccc(-c5nnn[nH]5)cc4)cc3)c2)CC1]

27.7 mg (23.9 μmol) of tetrakis(triphenylphosphine)palladium(0), 76.1 mg (0.72 mmol) of sodium carbonate and 0.36 ml (20.0 mmol) of water were added to a solution of 150 mg (0.24 mmol) of 4-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 102.2 mg (0.36 mmol) of {3-[(4-methylpiperazin-1-yl)carbonyl]phenyl}boronic acid hydrochloride in 1.8 ml of DMSO. The mixture was treated in the microwave at 110° C. for 1.5 h. The mixture was filtered and purified by preparative HPLC (Method 10). 52 mg (29% of theory) of the title compound were obtained.

LC-MS (Method 4): Rₜ=0.92 min; MS (ESIpos): m/z=750 [M+H−HCOOH]⁺.

### Example 20A

### tert-Butyl [trans-4-({(2S)-3-[2′-methyl-5′-(pyrrolidin-1-ylsulphonyl)biphenyl-4-yl]-1-oxo-1-[(2-oxo-2,3-dihydro-1H-benzimidazol-5-yl)amino]propan-2-yl 1 carbamoyl)cyclohexyl]methyl}carbamate

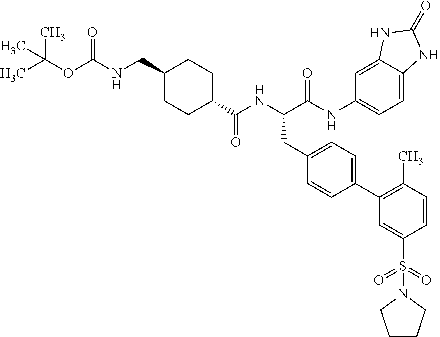 [SMILES: Cc1ccc(S(=O)(=O)N2CCCC2)cc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

28.2 mg (24.4 μmol) of tetrakis(triphenylphosphine)palladium(0), 77.6 mg (0.7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 100.5 mg (0.37 mmol) of [2-methyl-5-(pyrrolidin-1-ylsulphonyl)phenyl]boronic acid in 1.8 ml of DMSO. The mixture was treated in the microwave at 110° C. for 90 min. The mixture was filtered and purified by preparative HPLC (Method 10). 74 mg (40% of theory) of the title compound were obtained.

LC-MS (Method 4): Rₜ=1.22 min; MS (ESIpos): m/z=759 [M+H]⁺.

### Example 21A

### tert-Butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-methylbiphenyl-3-yl}carbonyl)amino]piperidine-1-carboxylate

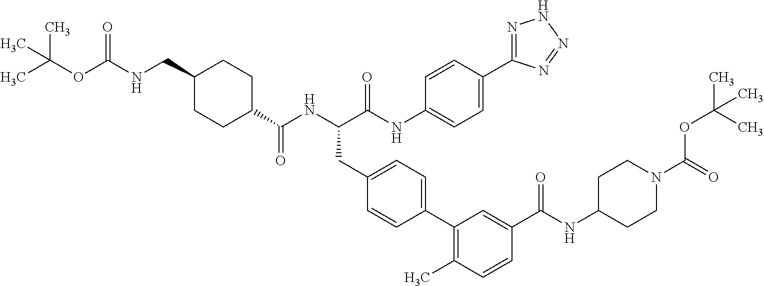

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-6-methylbiphenyl-3-carboxylic acid and 35 mg (0.18 mmol) of tert-butyl 4-aminopiperidine-1-carboxylate in 5 ml of tetrahydrofuran, and the mixture was stirred at RT for 16 h. The reaction mixture was separated directly by preparative HPLC (mobile phase: gradient of acetonitrile/water with 0.1% trifluoroacetic acid). This gave 69 mg of a mixture of the title compound and the partially deprotected title compound, which was used directly in the next stage.

LC-MS (Method 1): Rₜ=1.18 min; MS (ESIneg): m/z=862 [M−H]⁻.

### Example 22A

### tert-Butyl [(trans-4-{[(2S)-3-(5′-{[2-(diethylamino)ethyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate

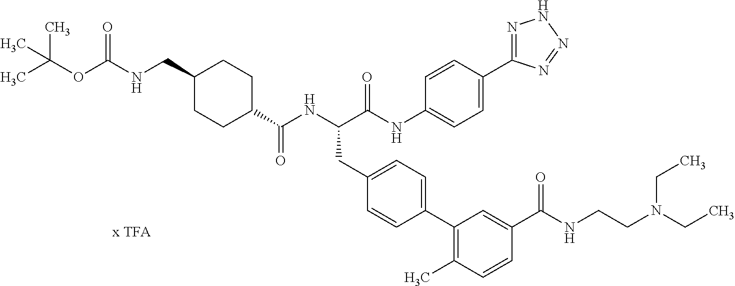

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-6-methylbiphenyl-3-carboxylic acid and 20 mg (0.18 mmol) of diethylaminoethylamine in 5 ml of tetrahydrofuran, and the mixture was stirred at RT for 40 h. The reaction mixture was separated directly by preparative HPLC (mobile phase: gradient of acetonitrile/water with 0.1% trifluoroacetic acid). This gave 64 mg of a mixture of the title compound and the partially deprotected title compound, which was used directly in the next stage.

LC-MS (Method 1): Rₜ=0.84 min; MS (ESIneg): m/z=778 [M−H-TFA]⁻.

### Example 23A

### tert-Butyl (3R)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-6-methylbiphenyl-3-yl}carbonyl)amino]pyrrolidine-1-carboxylate

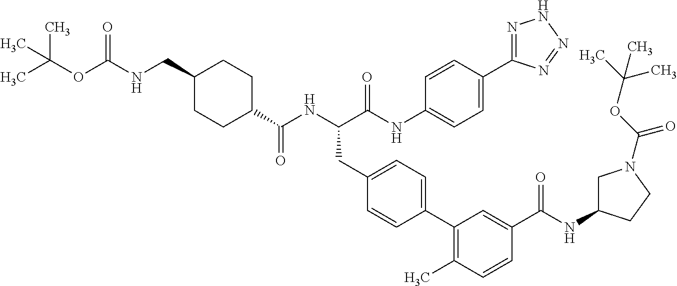

A solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-6-methylbiphenyl-3-carboxylic acid and 33 mg (0.18 mmol) of (R)-(+)-1-(tert-butoxycarbonyl)-3-aminopyrrolidine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 40 h. The reaction mixture was separated directly by preparative HPLC (mobile phase: gradient of acetonitrile/water with 0.1% trifluoroacetic acid). This gave 65 mg of a mixture of the title compound and the partially deprotected title compound, which was used directly in the next stage.

LC-MS (Method 1): Rₜ=1.16 min; MS (ESIneg): m/z=848 [M−H]⁻.

### Example 24A

### tert-Butyl 4-({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-6-methylbiphenyl-3-yl}carbonyl)piperazine-1-carboxylate

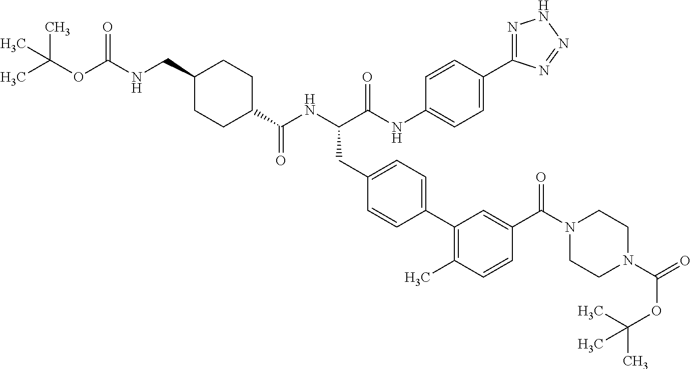

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-methylbiphenyl-3-carboxylic acid and 33 mg (0.18 mmol) of 1-(tert-butoxycarbonyl)piperazine in 5 ml of tetrahydrofuran, and the mixture was stirred at RT for 16 h. The reaction mixture was separated directly by preparative HPLC (mobile phase: gradient of acetonitrile/water with 0.1% trifluoroacetic acid). This gave 79 mg of a mixture of the title compound and the partially deprotected title compound, which was used directly in the next stage.

LC-MS (Method 1): Rₜ=1.16 min; MS (ESIneg): m/z=848 [M−H]⁻.

### Example 25A

### tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]biphenyl-3-yl}carbonyl)-amino]piperidine-1-carboxylate

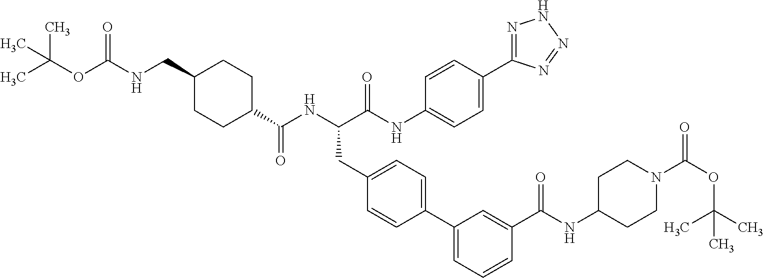

68 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]biphenyl-3-carboxylic acid and 36 mg (0.18 mmol) of tert-butyl 4-aminopiperidine-1-carboxylate in 5 ml of tetrahydrofuran, and the mixture was stirred at RT for 16 h. The reaction mixture was separated directly by preparative HPLC (mobile phase: gradient of acetonitrile/water with 0.1% trifluoroacetic acid). This gave 90 mg of a mixture of the title compound and the partially deprotected title compound, which was used directly in the next stage.

LC-MS (Method 1): Rₜ=1.15 min; MS (ESIneg): m/z=848 [M−H]⁻.

### Example 26A

### tert-Butyl [(trans-4-{[(2S)-3-(3′-{[2-(diethylamino)ethyl]carbamoyl biphenyl-4-yl)-1-oxo-1-{[4-(2H-tetrazol-5-yl)phenyl]amino}propan-2-yl]carbamoyl}cyclohexyl)methyl]carbamate trifluoroacetate

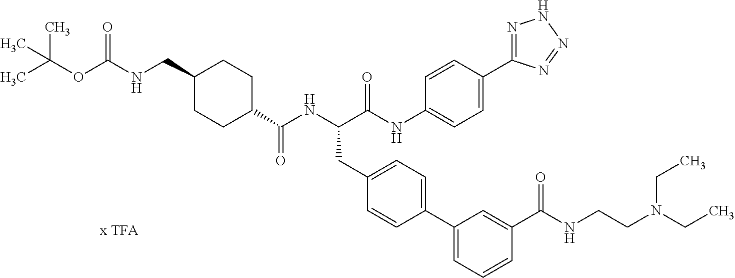

68 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-[4-(2H-tetrazol-5-yl)phenyl]amino}propyl]biphenyl-3-carboxylic acid and 21 mg (0.18 mmol) of diethylaminoethylamine in 5 ml of tetrahydrofuran, and the mixture was stirred at RT for 40 h. The reaction mixture was separated directly by preparative HPLC (mobile phase: gradient of acetonitrile/water with 0.1% trifluoroacetic acid). This gave 66 mg of a mixture of the title compound and the partially deprotected title compound, which was used directly in the next stage.

LC-MS (Method 1): Rₜ=0.83 min; MS (ESIneg): m/z=764 [M−H-TFA]⁻.

### Example 27A

### tert-Butyl 4-[({4′-[(2S)-2-{[(trans-4-{[(tert-butoxycarbonyl)amino]methyl}cyclohexyl) carbonyl]-amino}-3-oxo-3-{[4-(2H-tetrazol-5-yl)phenyl]amino}propyl]-2-fluorobiphenyl-3-yl}carbonyl)-amino]piperidine-1-carboxylate

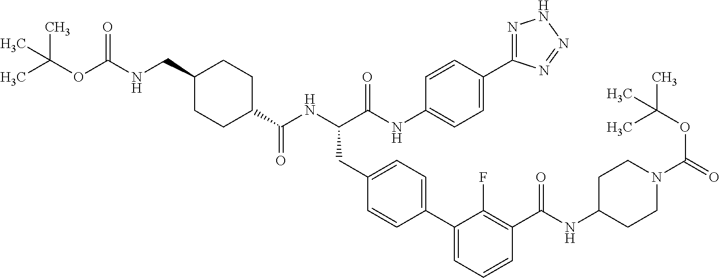

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-2-fluorobiphenyl-3-carboxylic acid and 35 mg (0.18 mmol) of tert-butyl 4-aminopiperidine-1-carboxylate in 5 ml of tetrahydrofuran, and the mixture was stirred at RT for 16 h. The reaction mixture was separated directly by preparative HPLC (mobile phase: gradient of acetonitrile/water with 0.1% trifluoroacetic acid). This gave 67 mg of a mixture of the title compound and the partially deprotected title compound, which was used directly in the next stage.

LC-MS (Method 1): Rₜ=1.13 min; MS (ESIneg): m/z=866 [M−H]⁻.

### Example 28A

### tert-Butyl [(trans-4-{[(2S)-3-(3′-{[2-(diethylamino)ethyl]carbamoyl}-2′-fluorobiphenyl-4-yl)-1-oxo-1-{[4-(2H-tetrazol-5-yl)phenyl]amino}propan-2-yl]carbamoyl}cyclohexyl)methyl]carbamate trifluoroacetate

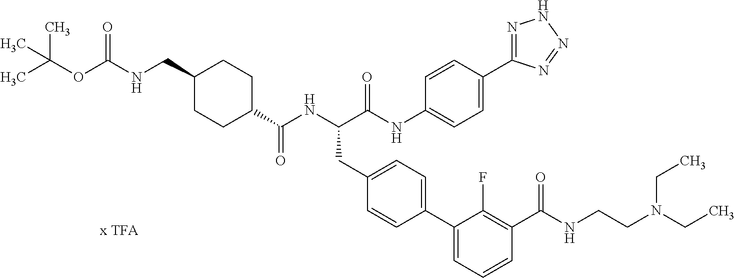

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-2-fluorobiphenyl-3-carboxylic acid and 20 mg (0.18 mmol) of diethylaminoethylamine in 5 ml of tetrahydrofuran, and the mixture was stirred at RT for 16 h. The reaction mixture was separated directly by preparative HPLC (mobile phase: gradient of acetonitrile/water with 0.1% trifluoroacetic acid). This gave 83 mg of a mixture of the title compound and the partially deprotected title compound, which was used directly in the next stage.

LC-MS (Method 1): Rₜ=0.87 min; MS (ESIneg): m/z=782 [M−H-TFA]⁻.

### Example 29A

### tert-Butyl (3R)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-fluorobiphenyl-3-yl}carbonyl)amino]pyrrolidine-1-carboxylate

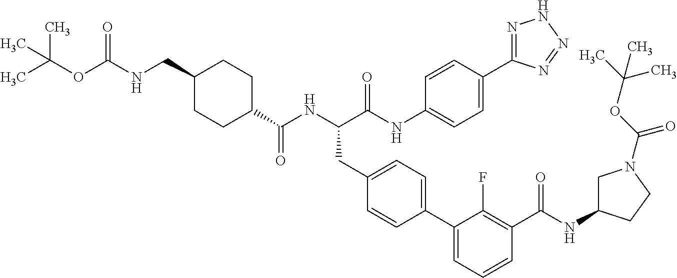

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-2-fluorobiphenyl-3-carboxylic acid and 33 mg (0.18 mmol) of (R)-(+)-1-(tert-butoxycabonyl)-3-aminopyrrolidine in 5 ml of tetrahydrofuran, and the mixture was stirred at RT for 16 h. The reaction mixture was separated directly by preparative HPLC (mobile phase: gradient of acetonitrile/water with 0.1% trifluoroacetic acid). This gave 71 mg of a mixture of the title compound and the partially deprotected title compound, which was used directly in the next stage.

LC-MS (Method 1): Rₜ=1.11 min; MS (ESIneg): m/z=852 [M−H]⁻.

### Example 30A

### tert-Butyl 4-({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-fluorobiphenyl-3-yl}carbonyl)piperazine-1-carboxylate

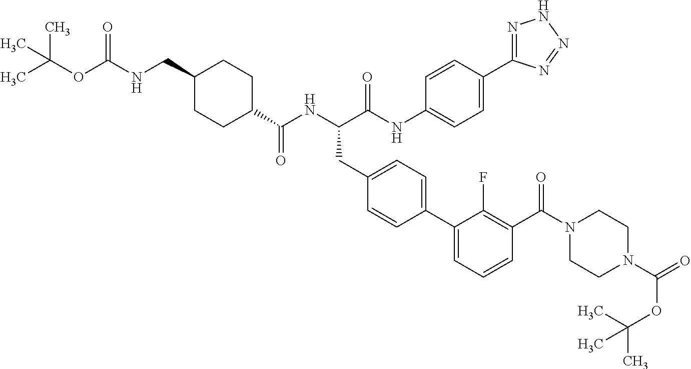

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-2-fluorobiphenyl-3-carboxylic acid and 33 mg (0.18 mmol) of 1-(tert-butoxycarbonyl)piperazine in 5 ml of tetrahydrofuran, and the mixture was stirred at RT for 16 h. The reaction mixture was separated directly by preparative HPLC (mobile phase: gradient of acetonitrile/water with 0.1% trifluoroacetic acid). This gave 63 mg of a mixture of the title compound and the partially deprotected title compound, which was used directly in the next stage.

LC-MS (Method 1): Rₜ=1.12 min; MS (ESIneg): m/z=852 [M−H]⁻.

### Example 31A

### tert-Butyl [(trans-4-{[(2S)-3-(5′-{[3-(diethylamino)propyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate

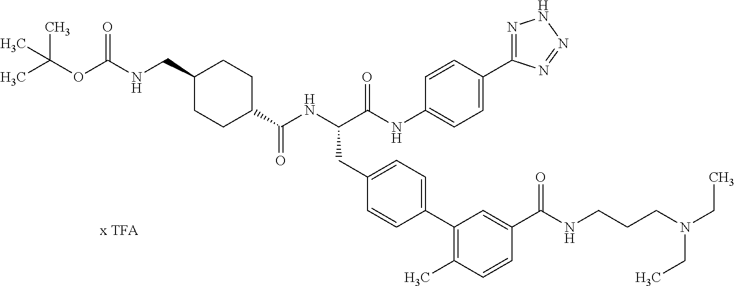

67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added to a solution of 100 mg (0.15 mmol) of 4′-[(2S)-2-{[(trans-4-{[(tert-butoxycarbonyl)amino]methyl}cyclohexyl) carbonyl]amino}-3-oxo-3-{[4-(2H-tetrazol-5-yl)phenyl]amino}propyl]-6-methylbiphenyl-3-carboxylic acid and 23 mg (0.18 mmol) of diethylaminopropylamine in 5 ml of tetrahydrofuran, and the mixture was stirred at RT for 16 h. After addition of 0.4 ml of N,N-dimethylformamide, the mixture was stirred at RT for a further 3 d. The reaction mixture was separated twice by preparative HPLC (mobile phase: gradient of acetonitrile/water with 0.1% trifluoroacetic acid). This gave 77 mg of a mixture of the title compound and the partially deprotected title compound, which was used directly in the next stage.

LC-MS (Method 1): Rₜ=0.89 min; MS (ESIneg): m/z=793 [M−H-TFA]⁻.

### Example 32A

### tert-Butyl [(trans-4-{[(2S)-3-[3′-(morpholin-4-ylcarbonyl)biphenyl-4-yl]-1-oxo-1-{[4-(2H-tetrazol-5-yl)phenyl]amino}propan-2-yl]carbamoyl}cyclohexyl)methyl]carbamate

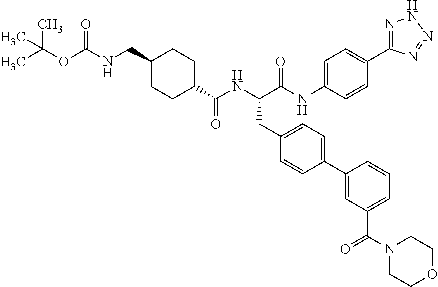

0.36 ml (0.72 mmol) of a 2M sodium carbonate solution in water was added to a solution of 150 mg (0.24 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide and 84 mg (0.36 mmol) of [3-(morpholin-4-ylcarbonyl)phenyl]boronic acid in 2.5 ml DMF, and the mixture was degassed with argon for 5 min 17.5 mg (0.02 mmol) of 1,1′-bis(diphenylphosphino)ferrocenepalladium(II) chloride were added and the mixture was stirred at 120° C. in a preheated oil bath for 30 min. The reaction solution was separated by preparative HPLC (mobile phase: gradient of acetonitrile/water with 0.1% trifluoroacetic acid). The product-containing fractions were combined and concentrated on a rotary evaporator. The residue was dried under high vacuum. 109 mg (56% of theory, 91% purity) of the title compound were obtained.

¹H-NMR (400 MHz, DMSO-d₆) δ=0.83 (m, 2H), 1.05-1.44 (m, 13H), 1.68 (m, 4H), 2.03-2.20 (m, 1H), 2.75 (m, 2H), 2.87-2.99 (m, 1H), 3.04-3.16 (m, 1H), 3.25-3.45 (m, 3H), 4.62-4.77 (m, 1H), 6.78 (m, 1H), 7.28-7.88 (m, 12H), 8.00 (d, 2H), 8.20 (d, 1H), 10.49 (s, 1H).

LC-MS (Method 1): Rₜ=0.97 min; MS (ESIneg): m/z=735 [M−H]⁻.

### Example 33A

### tert-Butyl 4-({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-4-methylbiphenyl-3-yl}carbamoyl)piperidine-1-carboxylate

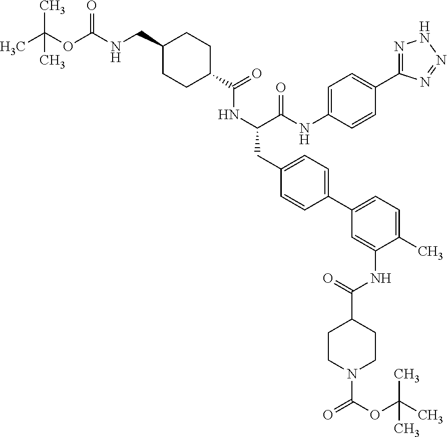 [SMILES: Cc1ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1NC(=O)C1CCN(C(=O)OC(C)(C)C)CC1]

64 μl (0.37 mmol) of N,N-diisopropylethylamine and 69.9 mg (0.18 mmol) of N-[(dimethylamino) (3H-[1,2,3]triazolo[4,5-b]pyridin-3-yloxy)methylidene]-N-methylmethanaminium hexafluorophosphate were added to a solution of 80 mg (0.12 mmol) of tert-butyl [(trans-4-{[(2S)-3-(3′-amino-4′-methylbiphenyl-4-yl)-1-oxo-1-{[4-(2H-tetrazol-5-yl)phenyl]amino}propan-2-yl]carbamoyl}cyclohexyl)methyl]carbamate and 56.2 mg (0.25 mmol) of 1-(tert-butoxycarbonyl)piperidine-4-carboxylic acid in 1 ml of DMF, and the mixture was stirred at RT for 16 h. The contents of the flask were diluted with water and acetonitrile and separated by preparative HPLC (mobile phase: gradient of acetonitrile/water with 0.1% trifluoroacetic acid). The product-containing fractions were combined and concentrated on a rotary evaporator. The residue was dried under high vacuum. 18 mg (15% of theory) of the title compound were obtained.

LC-MS (Method 1): Rₜ=1.16 min; MS (ESIpos): m/z=864 [M+H]⁺.

### Example 34A

### tert-Butyl [(trans-4-{[(2S)-3-(5′-{[2-(diethylamino)ethyl]carbamoyl}-2′-ethylbiphenyl-4-yl)-1-oxo-1-{[4-(2H-tetrazol-5-yl)phenyl]amino}propan-2-yl]carbamoyl}cyclohexyl)methyl]carbamate trifluoroacetate

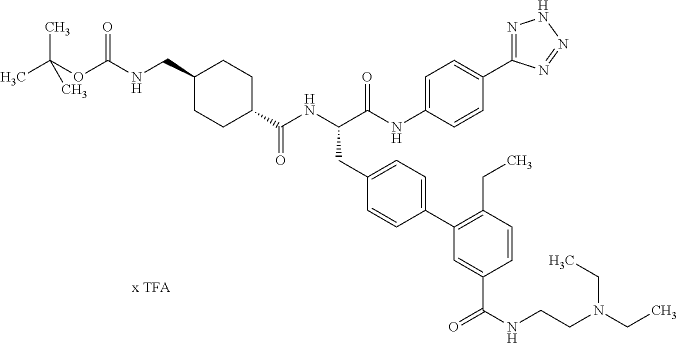 [SMILES: CCc1ccc(C(=O)NCCN(CC)CC)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

75 al (0.43 mmol) of N,N-diisopropylethylamine and 65.6 mg (0.17 mmol) of N-[(dimethylamino) (3H-[1,2,3]triazolo[4,5-b]pyridin-3-yloxy)methylidene]-N-methylmethanaminium hexafluorophosphate were added to a solution of 100 mg (0.14 mmol) of 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-ethylbiphenyl-3-carboxylic acid and 20 mg (0.17 mmol) of N,N-diethylethane-1,2-diamine in 3 ml of DMF, and the mixture was stirred at RT for 16 h. The contents of the flask were diluted with water and acetonitrile and separated by preparative HPLC (mobile phase: gradient of acetonitrile/water with 0.1% trifluoroacetic acid). The product-containing fractions were combined and concentrated on a rotary evaporator. The residue was dried under high vacuum. 63 mg (50% of theory, 94% purity) of the title compound were obtained.

LC-MS (Method 1): Rₜ=0.85 min; MS (ESIpos): m/z=794 [M+H]⁺.

### Example 35A

### tert-Butyl [(trans-4-{[(2S)-1-oxo-3-(3′-sulphamoylbiphenyl-4-yl)-1-{[4-(1H-tetrazol-5-yl)phenyl]-amino}propan-2-yl]carbamoyl}cyclohexyl)methyl]carbamate

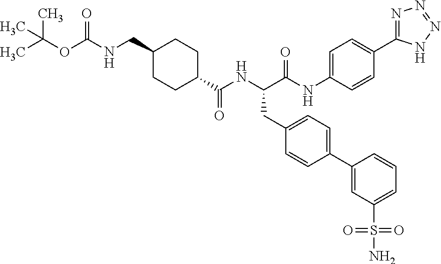 [SMILES: CC(C)(C)OC(=O)NC[C@H]1CC[C@H](C(=O)N[C@@H](Cc2ccc(-c3cccc(S(N)(=O)=O)c3)cc2)C(=O)Nc2ccc(-c3nnn[nH]3)cc2)CC1]

27.7 mg (23.9 μmol) of tetrakis(triphenylphosphine)palladium(0), 76.1 mg (0.72 mmol) of sodium carbonate and 0.36 ml (20.0 mmol) of water were added to a solution of 150 mg (0.24 mmol) of 4-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 72.2 mg (0.36 mmol) of (3-sulphamoylphenyl)boronic acid in 1.8 ml of DMSO. The mixture was treated in the microwave at 110° C. for 2 h. The mixture was filtered and purified by preparative HPLC (basic). 91 mg (54% of theory) of the title compound were obtained.

LC-MS (Method 5): Rₜ=0.76 min; MS (ESIpos): m/z=703 [M+H]⁺.

### Example 36A

### tert-Butyl [(trans-4-{[(2S)-3-{3-′-[2-(morpholin-4-yl)ethoxy]biphenyl-4-yl}-1-oxo-1-{[4-(2H-tetrazol-5-yl)phenyl]amino}propan-2-yl]carbamoyl}cyclohexyl)methyl]carbamate trifluoroacetate

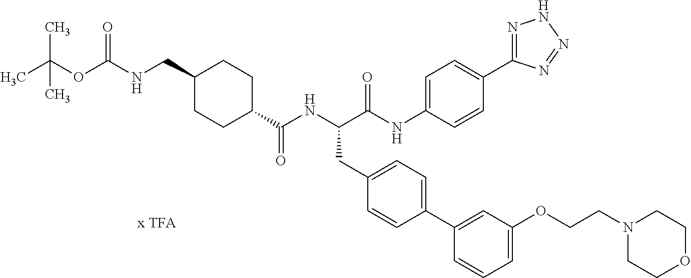 [SMILES: CC(C)(C)OC(=O)NC[C@H]1CC[C@H](C(=O)N[C@@H](Cc2ccc(-c3cccc(OCCN4CCOCC4)c3)cc2)C(=O)Nc2ccc(-c3nn[nH]n3)cc2)CC1]

18.4 mg (0.016 mmol) of tetrakis(triphenylphosphine)palladium(0) and 1.2 ml of 2M sodium carbonate solution in water were added to a solution of 100 mg (0.16 mmol) of 4-bromo-N-alpha-[(trans-4-{[(tert-butoxycarbonyl)amino]methyl}cyclohexyl) carbonyl]-N-[4-(2H-tetrazol-5-yl)phenyl]-L-phenylalaninamide and 133.0 mg (0.4 mmol) of 4-{2-[3-(4,4,5,5-tetramethyl-1,3,2-dioxaborolan-2-yl)phenoxy]ethyl}morpholine in 1.5 ml of 1,2-dimethoxyethane and 0.5 ml of ethanol. The mixture was stirred at 100° C. for 3 h. The mixture was filtered through kieselguhr and the filtrate was separated by preparative HPLC (mobile phase: gradient of acetonitrile/water with 0.1% trifluoroacetic acid). The product-containing fractions were combined and dried under high vacuum. 112 mg (63% of theory, 77% purity) of the title compound were obtained.

LC-MS (Method 1): Rₜ=0.84 min; MS (ESIpos): m/z=753 [M+H-TFA]⁺.

### Example 37A

### tert-Butyl [(trans-4-{[(2S)-3-(3′-amino-4′-methylbiphenyl-4-yl)-1-oxo-1-{[4-(2H-tetrazol-5-yl)phenyl]amino}propan-2-yl]carbamoyl}cyclohexyl)methyl]carbamate

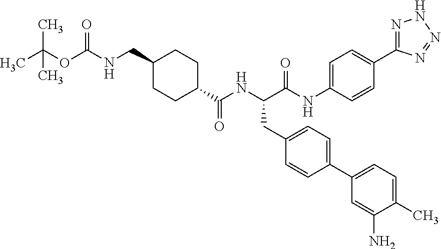 [SMILES: Cc1ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1N]

65.17 mg (0.08 mmol) of [1,1-bis(diphenylphosphino)ferrocene]dichloropalladium dichloromethane complex and 1.6 ml of 2M sodium carbonate solution in water were added to a solution of 1000 mg (1.60 mmol) of 4-bromo-N-alpha-[(trans-4-{[(tert-butoxycarbonyl)amino]methyl}cyclohexyl) carbonyl]-N-[4-(2H-tetrazol-5-yl)phenyl]-L-phenylalaninamide and 289.1 mg (1.92 mmol) of (3-amino-4-methylphenyl)boronic acid in 16 ml of 1,2-dimethoxyethane. The mixture was stirred at reflux for 2 h. The mixture was filtered, concentrated and recrystallized from hot acetonitrile. The precipitate was filtered off with suction, washed with a little cold acetonitrile and dried under high vacuum. 404 mg (39% of theory) of the title compound were obtained.

LC-MS (Method 1): Rₜ=1.03 min; MS (ESIpos): m/z=653 [M+H]⁺.

### Example 38A

### Ethyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-ethylbiphenyl-3-carboxylate

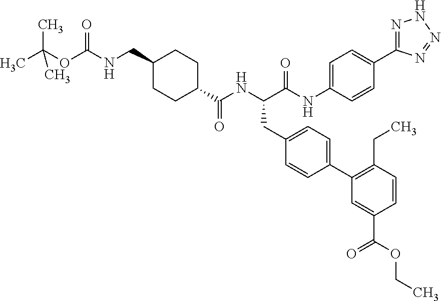 [SMILES: CCOC(=O)c1ccc(CC)c(-c2ccc(C[C@H](NC(=O)[C@H]3CC[C@H](CNC(=O)OC(C)(C)C)CC3)C(=O)Nc3ccc(-c4nn[nH]n4)cc3)cc2)c1]

182.4 mg (0.48 mmol) of bis(pinacolato)diborane, 172.4 mg (0.67 mmol) of ethyl 3-bromo-4-ethylbenzoate and 140.9 mg (1.44 mmol) of potassium acetate were initially charged in 4 ml of toluene under argon, 19.6 mg (24 μmol) of [1,1-bis(diphenylphosphino)ferrocene]dichloropalladium dichloromethane complex were added and the mixture was stirred at 110° C. for 3 h. 300 mg (0.48 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 19.6 mg (24 μmol) of 1,1-bis-(diphenylphosphino)ferrocene]dichloropalladium dichloromethane complex, 1.5 ml of ethanol and 0.48 ml (0.96 mmol) of 2M sodium carbonate solution in water were added to the mixture. The mixture was stirred at 110° C. for 6 h and then at RT overnight. The mixture was separated by preparative HPLC (mobile phase: gradient of acetonitrile/water with 0.1% trifluoroacetic acid). The product-containing fractions were combined and concentrated on a rotary evaporator. The residue was dried under high vacuum. 107 mg (31% of theory) of the title compound were obtained.

LC-MS (Method 1): Rₜ=1.23 min; MS (ESIpos): m/z=724 [M+H]⁺.

### Example 39A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-6-ethylbiphenyl-3-carboxylic acid

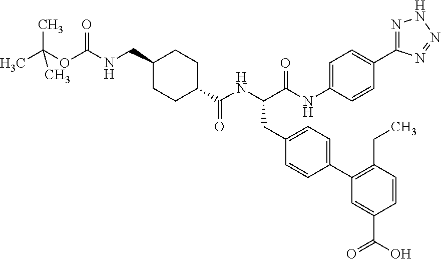 [SMILES: CCc1ccc(C(=O)O)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

At RT, 60.9 mg (1.45 mmol) of lithium hydroxide were added to a solution of 105 mg (0145 μmol) of ethyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-ethylbiphenyl-3-carboxy late, 3 ml of tetrahydrofuran and 1 ml of water. The reaction mixture was stirred at RT for 4 days, the pH was then adjusted to 5-6 using 1M hydrochloric acid solution and the mixture was extracted with ethyl acetate. The organic phase was dried over sodium sulphate and concentrated. 102 mg (80% of theory, 79% purity) of the title compound were obtained.

LC-MS (Method 1): Rₜ=1.05 min; MS (ESIneg): m/z=696 [M+H]⁺.

### Example 40A

### tert-Butyl [(trans-4-{[(2S)-3-(5′-cyano-2′-methylbiphenyl-4-yl)-1-oxo-1-{[4-(2H-tetrazol-5-yl)phenyl]amino}propan-2-yl]carbamoyl}cyclohexyl)methyl]carbamate

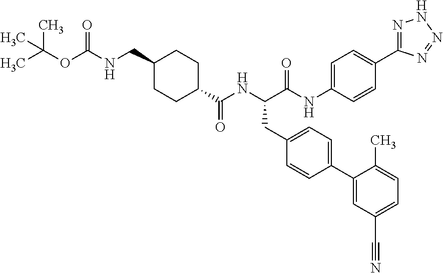 [SMILES: Cc1ccc(C#N)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

0.36 ml (0.72 mmol) of a 2M sodium carbonate solution in water was added to a solution of 150 mg (0.24 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide and 87.3 mg (0.36 mmol) of (5-cyano-2-methylphenyl)boronic acid in 2.5 ml DMF, and the mixture was degassed with argon for 5 min 17.5 mg (0.02 mmol) of 1,1′-bis(diphenylphosphino)ferrocenepalladium(II) chloride were added and the mixture was stirred at 120° C. in a preheated oil bath for 2 h. The mixture was filtered through kieselguhr and washed through with ethyl acetate. The filtrate was diluted with ethyl acetate and water, acidified with 10% strength citric acid and once more washed with ethyl acetate. The organic phase of the filtrate was dried over magnesium sulphate and concentrated under reduced pressure. The residue was suspended in dichloromethane, the resulting solid was precipitated and filtered, washed with acetonitrile and dried under high vacuum. 86 mg (51% of theory) of the title compound were obtained.

LC-MS (Method 1): Rₜ=1.11 min; MS (ESIpos): m/z=663 [M+H]⁺.

### Example 41A

### tert-Butyl [(trans-4-{[(2S)-3-[4′-methoxy-3′-(morpholin-4-ylsulphonyl)biphenyl-4-yl]-1-oxo-1-{[4-(1H-tetrazol-5-yl)phenyl]amino}propan-2-yl]carbamoyl}cyclohexyl)methyl]carbamate

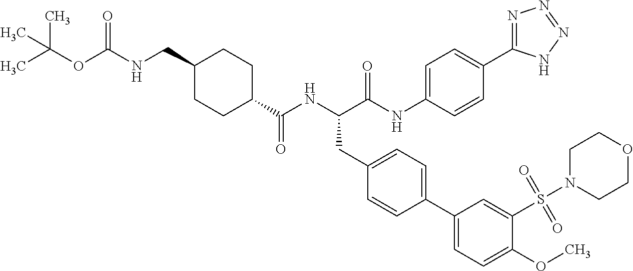 [SMILES: COc1ccc(-c2ccc(C[C@H](NC(=O)[C@H]3CC[C@H](CNC(=O)OC(C)(C)C)CC3)C(=O)Nc3ccc(-c4nnn[nH]4)cc3)cc2)cc1S(=O)(=O)N1CCOCC1]

27.7 mg (23.9 μmol) of tetrakis(triphenylphosphine)palladium(0), 76.1 mg (0.72 mmol) of sodium carbonate and 0.36 ml (20.0 mmol) of water were added to a solution of 150 mg (0.24 mmol) of 4-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 108.2 mg (0.36 mmol) of [4-methoxy-3-(morpholin-4-ylsulphonyl)phenyl]boronic acid in 1.8 ml of DMSO. The mixture was treated in the microwave at 110° C. for 2.5 h. The mixture was filtered and purified by preparative HPLC (Method 10). 86 mg (45% of theory) of the title compound were obtained.

LC-MS (Method 4): Rₜ=1.20 min; MS (ESIpos): m/z=803 [M+H]⁺.

### Example 42A

### tert-Butyl {[trans-4-({(2S)-3-[5′-(dimethylsulphamoyl)-2′-methylbiphenyl-4-yl]-1-oxo-1-[(2-oxo-2,3-dihydro-1H-benzimidazol-5-yl)amino]propan-2-yl}carbamoyl)cyclohexyl]methyl}carbamate

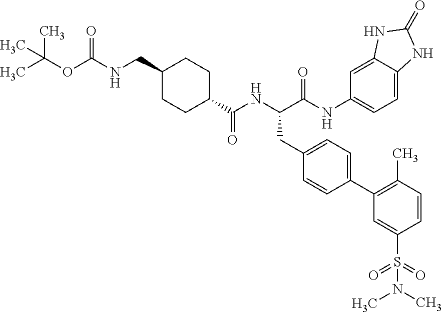 [SMILES: Cc1ccc(S(=O)(=O)N(C)C)cc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

28.2 mg (24.4 μmol) of tetrakis(triphenylphosphine)palladium(0), 77.6 mg (0.7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 90.8 mg (0.37 mmol) of [5-(dimethylsulphamoyl)-2-methylphenyl]boronic acid in 1.8 ml of DMSO. The mixture was treated in the microwave at 110° C. for 90 min. The mixture was filtered and purified by preparative HPLC (Method 10). 68 mg (38% of theory) of the title compound were obtained.

LC-MS (Method 4): Rₜ=1.18 min; MS (ESIpos): m/z=733 [M+H]⁺.

### Example 43A

### tert-Butyl {[trans-4-({(2S)-3-[2′-methyl-5′-(morpholin-4-ylsulphonyl)biphenyl-4-yl]-1-oxo-1-[(2-oxo-2,3-dihydro-1H-benzimidazol-5-yl)amino]propan-2-yl}carbamoyl)cyclohexyl]methyl}carbamate

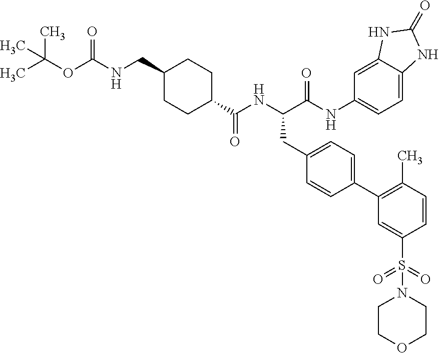 [SMILES: Cc1ccc(S(=O)(=O)N2CCOCC2)cc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

28.2 mg (24.4 μmol) of tetrakis(triphenylphosphine)palladium(0), 77.6 mg (0.7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 106.5 mg (0.37 mmol) of [2-methyl-5-(morpholin-4-ylsulphonyl)phenyl]boronic acid in 1.8 ml of DMSO. The mixture was treated in the microwave at 110° C. for 90 min. The mixture was filtered and purified by preparative HPLC (Method 10). 79 mg (42% of theory) of the title compound were obtained.

LC-MS (Method 4): Rₜ=1.17 min; MS (ESIpos): m/z=775 [M+H]⁺.

### Example 44A

### tert-Butyl [(trans-4-{[(2S)-3-(3′-{[2-(morpholin-4-yl)ethyl]carbamoyl}biphenyl-4-yl)-1-oxo-1-{[4-(1H-tetrazol-5-yl)phenyl]amino}propan-2-yl]carbamoyl}cyclohexyl)methyl]carbamate formate

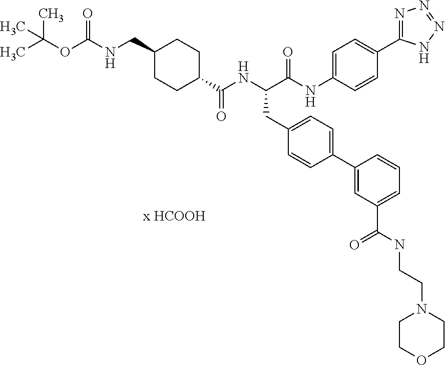 [SMILES: CC(C)(C)OC(=O)NC[C@H]1CC[C@H](C(=O)N[C@@H](Cc2ccc(-c3cccc(C(=O)NCCN4CCOCC4)c3)cc2)C(=O)Nc2ccc(-c3nnn[nH]3)cc2)CC1]

27.7 mg (23.9 μmol) of tetrakis(triphenylphosphine)palladium(0), 76.1 mg (0.72 mmol) of sodium carbonate and 0.36 ml (20.0 mmol) of water were added to a solution of 150 mg (0.24 mmol) of 4-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 123.0 mg (0.36 mmol) of (3-{[2-(morpholin-4-yl)ethyl]carbamoyl}phenyl)boronic acid hydrochloride in 1.8 ml of DMSO. The mixture was treated in the microwave at 110° C. for 2.5 h. The mixture was filtered and purified by preparative HPLC (Method 10). 60 mg (32% of theory) of the title compound were obtained.

LC-MS (Method 4): Rₜ=0.94 min; MS (ESIpos): m/z=780 [M+H-HCOOH]⁺.

### Example 45A

### tert-Butyl [(trans-4-{[(2S)-3-[5′-(dimethylamino)-2′-methylbiphenyl-4-yl]-1-oxo-1-{[4-(1H-tetrazol-5-yl)phenyl]amino}propan-2-yl]carbamoyl}cyclohexyl)methyl]carbamate formate

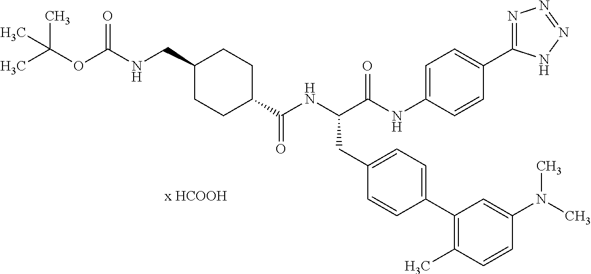 [SMILES: Cc1ccc(N(C)C)cc1-c1ccc(C[C@H](NC(=O)[C@H]2CC[C@H](CNC(=O)OC(C)(C)C)CC2)C(=O)Nc2ccc(-c3nnn[nH]3)cc2)cc1]

27.7 mg (23.9 μmol) of tetrakis(triphenylphosphine)palladium(0), 76.1 mg (0.72 mmol) of sodium carbonate and 0.36 ml (20.0 mmol) of water were added to a solution of 150 mg (0.24 mmol) of 4-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 64.3 mg (0.36 mmol) of [5-(dimethylamino)-2-methylphenyl]boronic acid in 1.8 ml of DMSO. The mixture was treated in the microwave at 110° C. for 1.5 h. The mixture was filtered and purified by preparative HPLC (Method 11). 30 mg (18% of theory) of the title compound were obtained.

LC-MS (Method 5): Rₜ=0.94 min; MS (ESIpos): m/z=681 [M+H-HCOOH]⁺.

### Example 46A

### tert-Butyl [(trans-4-{[(2S)-3-{4′-methyl-3′-[(tetrahydro-2H-pyran-4-ylcarbonyl)amino]biphenyl-4-yl}-1-oxo-1-{[4-(2H-tetrazol-5-yl)phenyl]amino}propan-2-yl]carbamoyl}cyclohexyl)methyl]-carbamate

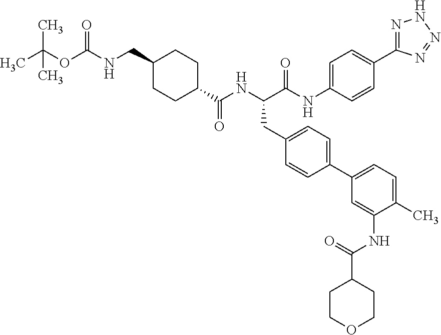 [SMILES: Cc1ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1NC(=O)C1CCOCC1]

66 μl (0.38 mmol) of N,N-diisopropylethylamine and 72.5 mg (0.19 mmol) of N-[(dimethylamino) (3H-[1,2,3]triazolo[4,5-b]pyridin-3-yloxy)methylidene]-N-methylmethanaminium hexafluorophosphate were added to a solution of 83 mg (0.13 mmol) of tert-butyl [(trans-4-{[(2S)-3-(3′-amino-4′-methylbiphenyl-4-yl)-1-oxo-1-{[4-(2H-tetrazol-5-yl)phenyl]amino}propan-2-yl]carbamoyl}cyclohexyl)methyl]carbamate and 33 mg (0.25 mmol) of tetrahydro-2H-pyran-4-carboxylic acid in 1 ml of DMF, and the mixture was stirred at RT for 16 h. The contents of the flask were diluted with water and acetonitrile and separated by preparative HPLC (mobile phase: gradient of acetonitrile/water with 0.1% trifluoroacetic acid). The product-containing fractions were combined and concentrated on a rotary evaporator. The residue was dried under high vacuum. This gave 24 mg (22% of theory) of the title compound.

LC-MS (Method 1): Rₜ=1.01 min; MS (ESIpos): m/z=765 [M+H]⁺.

### Example 47A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5-fluoro-6-methylbiphenyl-3-carboxylic acid

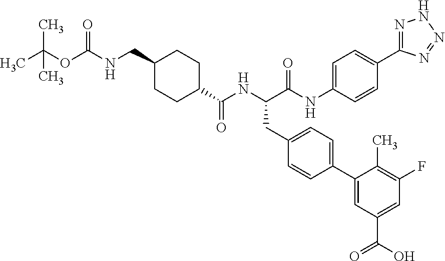

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (1.00 g, 1.60 mmol) from Example 6A, methyl 3-fluoro-4-methyl-5-(4,4,5,5-tetramethyl-1,3,2-dioxaborolan-2-yl)benzoate (704 mg, 2.39 mmol) and [2′-(azanidyl-kappa-N-)biphenyl-2-yl-kappaC2][di-(3s,5s,7s)-adamantan-1-yl(butyl)phosphoranyl](methanesulphonatato-kappa-O-)palladium (116 mg, 0.16 mmol) were initially charged under argon, 12.0 ml of methanol and 6.38 ml (6.38 mmol) of 1M aqueous potassium phosphate solution were then added and the reaction mixture was shaken at 65° C. overnight. At RT, the mixture was then diluted with 10 ml of water and filtered. The filtrate was acidified with 1M hydrochloric acid, and the solid formed was filtered off, washed with water and dried under high vacuum. Without further purification, the crude product obtained in this manner (1.07 g, mixture of title compound and corresponding methyl ester about 1:4) was taken up in 5.0 ml of THF, 6.0 ml of 1M aqueous lithium hydroxide solution were added and the mixture was stirred at RT overnight. The mixture was then diluted with water and acidified with 1M hydrochloric acid. The solid formed was filtered off, washed with water and dried under high vacuum. The solid was then triturated with methyl tert-butyl ether/ethyl acetate (2:1), filtered and dried under high vacuum. This gave 666 mg (90% pure) of the title compound which was reacted without further purification.

LC-MS (Method 1): Rₜ=1.02 min; MS (ESIpos): m/z=700 [M+H]⁺.

¹H-NMR (400 MHz, DMSO-d₆): δ=ppm 13.20 (br. s., 1H), 10.44 (s, 1H), 8.18 (d, 1H), 8.00 (d, 2H), 7.82 (d, 2H), 7.66-7.54 (m, 2H), 7.42 (d, 2H), 7.31 (d, 2H), 6.81-6.70 (m, 1H), 4.82-4.71 (m, 1H), 3.20-3.10 (m, 1H), 3.01-2.91 (m, 1H), 2.79-2.71 (m, 2H), 2.20-2.09 (m, 4H), 1.73-1.59 (m, 3H), 1.54 (d, 1H), 1.42-1.32 (m, 6H), 1.25 (d, 1H), 0.82 (d, 1H).

### Example 48A

### tert-Butyl [(trans-4-{[(2S)-3-[3′-fluoro-5′-(isopropylcarbamoyl)-2′-methylbiphenyl-4-yl]-1-oxo-1-{[4-(2H-tetrazol-5-yl)phenyl]amino}propan-2-yl]carbamoyl}cyclohexyl)methyl]carbamate

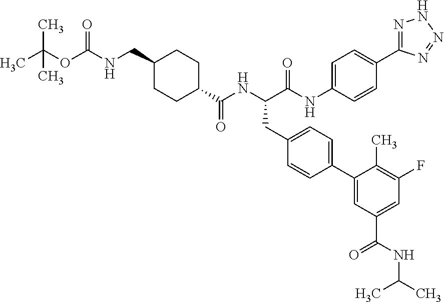 [SMILES: Cc1c(F)cc(C(=O)NC(C)C)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5-fluoro-6-methylbiphenyl-3-carboxylic acid (200 mg, 0.25 mmol) was initially charged in 3.5 ml of dimethylformamide, and isopropylamine (30 mg, 0.51 mmol), N,N-diisopropylethylamine (0.13 ml, 0.77 mmol) and 1-[bis(dimethylamino)methylene]-1H-1,2,3-triazolo[4,5-b]pyridinium 3-oxide hexafluorophosphate (147 mg, 0.39 mmol) were added. The reaction mixture was stirred at RT for another 1 h and allowed to stand at RT for 3 days. The reaction mixture was then diluted with water and acidified with 1M hydrochloric acid. The solid formed was filtered off, washed with in each case 10 ml of water and methyl tert-butyl ether and dried under high vacuum. The crude product was purified by preparative HPLC (mobile phase: acetonitrile/water with 0.1% formic acid (gradient)). This gave 71 mg (37% of theory) of the title compound.

LC-MS (Method 1): Rₜ=1.06 min; MS (ESIpos): m/z=741 [M+H]⁺.

¹H-NMR (400 MHz, DMSO-d₆): δ=ppm 16.67 (br. s., 1H), 10.43 (s, 1H), 8.29 (d, 1H), 8.17 (d, 1H), 7.99 (d, 2H), 7.82 (d, 2H), 7.64-7.54 (m, 2H), 7.41 (d, 2H), 7.31 (d, 2H), 6.81-6.72 (m, 1H), 4.81-4.69 (m, 1H), 4.16-4.00 (m, 1H), 3.20-3.07 (m, 1H), 3.01-2.89 (m, 1H), 2.79-2.72 (m, 2H), 2.18-2.05 (m, 4H), 1.74-1.49 (m, 4H), 1.37 (s, 9H), 1.29-1.20 (m, 2H), 1.15 (d, 7H), 0.91-0.74 (m, 2H).

### Example 49A

### tert-Butyl 4-[({4′-[(2S)-2-{[(trans-4-{[(tert-butoxycarbonyl)amino]methyl}cyclohexyl)-carbonyl]amino}-3-oxo-3-[4-(2H-tetrazol-5-yl)phenyl]amino}propyl]-5-fluoro-6-methylbiphenyl-3-yl}carbonyl)amino]piperidine-1-carboxylate

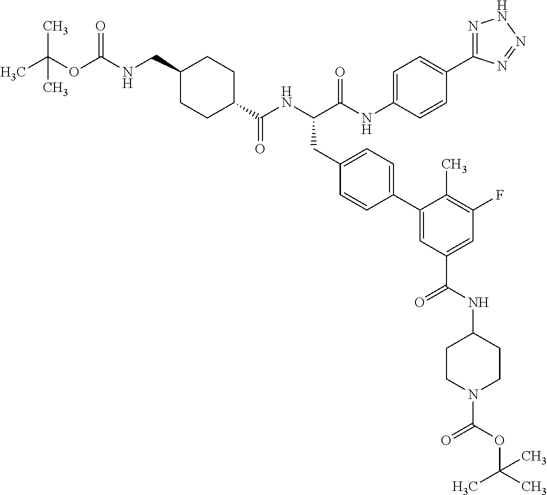 [SMILES: Cc1c(F)cc(C(=O)NC2CCN(C(=O)OC(C)(C)C)CC2)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5-fluoro-6-methylbiphenyl-3-carboxylic acid (200 mg, 0.25 mmol) was initially charged in 3.1 ml of dimethylformamide, and tert-butyl-4-aminopiperidine-1-carboxylate (92 mg, 0.46 mmol), N,N-diisopropylethylamine (0.12 ml, 0.69 mmol) and 1-[bis(dimethylamino)methylene]-1H-1,2,3-triazolo[4,5-b]pyridinium 3-oxide hexafluorophosphate (130 mg, 0.34 mmol) were added. The reaction mixture was stirred at RT for another 1 h and allowed to stand at RT for 3 days. The reaction mixture was then diluted with water and acidified with 1M hydrochloric acid. The solid formed was filtered off, washed with in each case 10 ml of water and methyl tert-butyl ether and dried under high vacuum. The crude product was purified by preparative HPLC (mobile phase: acetonitrile/water with 0.1% formic acid (gradient)). This gave 108 mg (53% of theory) of the title compound.

LC-MS (Method 1): Rₜ=1.15 min; MS (ESIpos): m/z=882 [M+H]⁺.

¹H-NMR (400 MHz, DMSO-d₆): δ=ppm 16.68 (br. s., 1H), 10.42 (s, 1H), 8.33 (d, 1H), 8.17 (d, 1H), 7.99 (d, 2H), 7.82 (d, 2H), 7.65-7.55 (m, 2H), 7.41 (d, 2H), 7.30 (d, 2H), 6.79-6.72 (m, 1H), 4.81-4.70 (m, 1H), 4.03-3.85 (m, 3H), 3.18-3.09 (m, 1H), 3.00-2.90 (m, 1H), 2.89-2.71 (m, 3H), 2.16-2.06 (m, 3H), 1.81-1.59 (m, 4H), 1.59-1.49 (m, 1H), 1.30-1.07 (m, 3H), 0.82 (br. s., 2H).

### Example 50A

### tert-Butyl [(trans-4-{[(2S)-3-{5′-[(trans-4-hydroxycyclohexyl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate

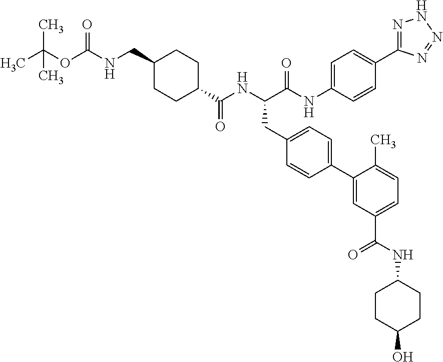 [SMILES: Cc1ccc(C(=O)N[C@H]2CC[C@H](O)CC2)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-6-methylbiphenyl-3-carboxylic acid (120 mg, 0.18 mmol) from Example 12A and trans-4-aminocyclohexanol (41 mg, 0.35 mmol) were initially charged in 3.0 ml of dimethylformamide, N,N-diisopropylethylamine (0.09 ml, 0.53 mmol) and 1-[bis(dimethylamino)methylene]-1H-1,2,3-triazolo[4,5-b]pyridinium 3-oxide hexafluorophosphate (100 mg, 0.26 mmol) were added and the mixture was allowed to stand at RT overnight. The reaction mixture was diluted with acetonitrile/water and filtered, and the filtrate was purified by preparative HPLC (mobile phase: acetonitrile/water with 0.1% formic acid (gradient)). This gave 136 mg (99% of theory) of the title compound.

LC-MS (Method 1): Rₜ=0.97 min; MS (ESIpos): m/z=779 [M+H]⁺.

¹H-NMR (400 MHz, DMSO-d₆): δ=ppm 10.42 (s, 1H), 8.20-8.10 (m, 2H), 7.99 (d, 2H), 7.82 (d, 2H), 7.74-7.70 (m, 1H), 7.66-7.64 (m, 1H), 7.39 (d, 2H), 7.33 (d, 1H), 7.28 (d, 2H), 6.80-6.72 (m, 1H), 4.78-4.69 (m, 1H), 4.58-4.46 (m, 1H), 3.77-3.65 (m, 1H), 3.16-3.08 (m, 1H), 2.99-2.90 (m, 1H), 2.80-2.71 (m, 2H), 2.20 (s, 3H), 2.16-2.06 (m, 1H), 1.88-1.74 (m, 4H), 1.73-1.51 (m, 4H), 1.37 (s, 9H), 1.34-1.11 (m, 6H), 0.90-0.75 (m, 2H).

### Example 51A

### tert-Butyl [(trans-4-{[(2S)-3-[2′-methyl-5′-(morpholin-4-ylcarbonyl)biphenyl-4-yl]-1-oxo-1-{[4-(2H-tetrazol-5-yl)phenyl]amino}propan-2-yl]carbamoyl}cyclohexyl)methyl]carbamate

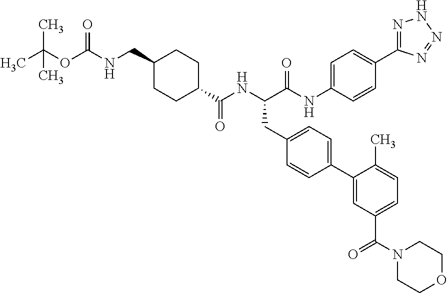 [SMILES: Cc1ccc(C(=O)N2CCOCC2)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-6-methylbiphenyl-3-carboxylic acid from Example 12A (130 mg, 0.19 mmol) and morpholine (0.03 ml, 0.38 mmol) were initially charged in 3.0 ml of dimethylformamide, N,N-diisopropylethylamine (0.10 ml, 0.57 mmol) and 1-[bis(dimethylamino)methylene]-1H-1,2,3-triazolo[4,5-b]pyridinium 3-oxide hexafluorophosphate (108 mg, 0.28 mmol) were added and the mixture was allowed to stand at RT overnight. The reaction mixture was diluted with acetonitrile/water and filtered, and the filtrate was purified by preparative HPLC (mobile phase: acetonitrile/water with 0.1% formic acid (gradient)). This gave 56 mg of the title compound as a crude product, which was converted without further purification.

LC-MS (Method 1): Rₜ=0.96 min; MS (ESIpos): m/z=752 [M+H]⁺.

### Example 52A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5,6-dimethylbiphenyl-3-carboxylic acid

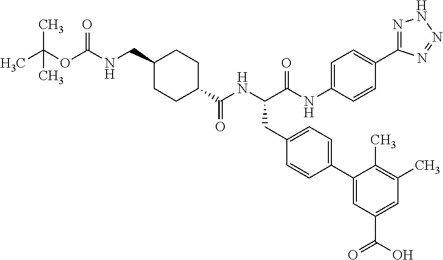 [SMILES: Cc1cc(C(=O)O)cc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1C]

3,4-Dimethyl-5-(4,4,5,5-tetramethyl-1,3,2-dioxaborolan-2-yl)benzoic acid (617 mg, 2.23 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (1.00 g, 1.60 mmol) from Example 6A and 507 mg (4.79 mmol) of sodium carbonate were initially charged in a mixture of 15.0 ml of DMF and 1.5 ml of water under argon. Subsequently, 1,1-bis(diphenylphosphino)ferrocenedichloropalladium(II) (130 mg, 0.16 mmol) was added and the mixture was stirred at 150° C. in a microwave for 1 h. Thereafter, the reaction mixture was diluted with 50 ml of water, acidified with 1M hydrochloric acid and extracted twice with 50 ml each time of diethyl ether. The combined organic phases were washed once each with water and saturated aqueous sodium chloride solution, dried over magnesium sulphate, filtered and concentrated. The residue was purified by preparative HPLC (mobile phase: acetonitrile/water with 0.1% formic acid (gradient)). This gave 299 mg of the title compound as a crude product, which was converted further without further purification.

LC-MS (Method 1): Rₜ=1.04 min; MS (ESIpos): m/z=696 [M+H]⁺.

### Example 53A

### tert-Butyl [(trans-4-{[(2S)-3-{2′,3′-dimethyl-5′-[(1-methylpiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]-carbamate

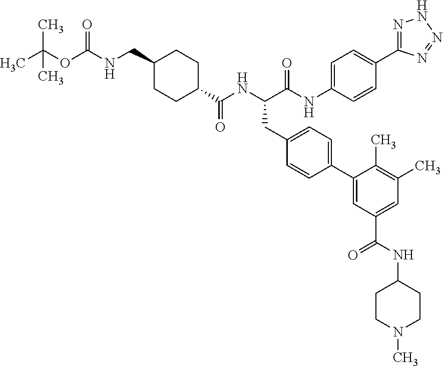 [SMILES: Cc1cc(C(=O)NC2CCN(C)CC2)cc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1C]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5,6-dimethylbiphenyl-3-carboxylic acid (150 mg, 0.22 mmol) and 4-amino-N-methylpiperidine (49 mg, 0.43 mmol) were initially charged in 3.0 ml of dimethylformamide, N,N-diisopropylethylamine (0.15 ml, 0.86 mmol) and 1-[bis(dimethylamino)methylene]-1H-1,2,3-triazolo[4,5-b]pyridinium 3-oxide hexafluorophosphate (123 mg, 0.32 mmol) were added and the mixture was allowed to stand at RT overnight. The reaction mixture was diluted with acetonitrile/water and filtered, and the filtrate was purified by preparative HPLC (mobile phase: acetonitrile/water with 0.1% formic acid (gradient)). This gave 80 mg (46% of theory) of the title compound.

LC-MS (Method 1): Rₜ=0.85 min; MS (ESIpos): m/z=792 [M+H]⁺.

¹H-NMR (400 MHz, DMSO-d₆): δ=ppm 10.15 (s, 1H), 8.29 (d, 1H), 8.18-8.09 (m, 1H), 7.92 (d, 2H), 7.68-7.59 (m, 3H), 7.49 (s, 1H), 7.37 (d, 2H), 7.22 (d, 2H), 6.82-6.73 (m, 1H), 4.79-4.69 (m, 1H), 3.99-3.85 (m, 1H), 3.30-3.15 (m, 4H), 3.15-3.08 (m, 2H), 2.99-2.90 (m, 1H), 2.82-2.70 (m, 4H), 2.59 (s, 3H), 2.33 (s, 3H), 2.18-2.12 (m, 1H), 2.09 (s, 3H), 1.90 (d, 2H), 1.76-1.54 (m, 6H), 1.37 (s, 9H), 1.33-1.10 (m, 3H), 0.91-0.75 (m, 2H).

### Example 54A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-4-fluorobiphenyl-3-carboxylic acid

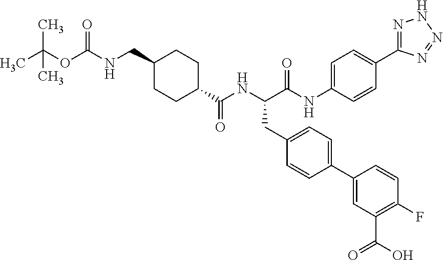 [SMILES: CC(C)(C)OC(=O)NC[C@H]1CC[C@H](C(=O)N[C@@H](Cc2ccc(-c3ccc(F)c(C(=O)O)c3)cc2)C(=O)Nc2ccc(-c3nn[nH]n3)cc2)CC1]

2-Fluoro-5-(4,4,5,5-tetramethyl-1,3,2-dioxaborolan-2-yl)benzoic acid (64 mg, 0.24 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (100 mg, 0.16 mmol) from Example 6A and 51 mg (0.48 mmol) of sodium carbonate were initially charged in a mixture of 2.0 ml of DMF and 0.2 ml of water under argon. Subsequently, 1,1-bis(diphenylphosphino)ferrocenedichloropalladium(II) (12 mg, 0.02 mmol) was added and the mixture was stirred at 150° C. in a microwave for 1 h. Thereafter, the reaction mixture was diluted with 20 ml of water, acidified with 1M hydrochloric acid and extracted twice with 20 ml each time of ethyl acetate. The combined organic phases were washed once each with water and saturated aqueous sodium chloride solution, dried over magnesium sulphate, filtered and concentrated. The residue was purified by preparative HPLC (mobile phase: acetonitrile/water with 0.1% formic acid (gradient)). This gave 38 mg of the title compound as a crude product, which was converted further without further purification.

LC-MS (Method 1): Rₜ=0.97 min; MS (ESIpos): m/z=686 [M+H]⁺.

### Example 55A

### tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-4-fluorobiphenyl-3-yl}carbonyl)-amino]piperidine-1-carboxylate

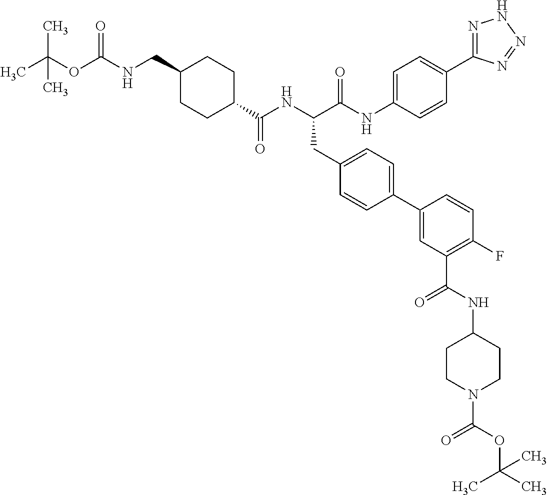 [SMILES: CC(C)(C)OC(=O)NC[C@H]1CC[C@H](C(=O)N[C@@H](Cc2ccc(-c3ccc(F)c(C(=O)NC4CCN(C(=O)OC(C)(C)C)CC4)c3)cc2)C(=O)Nc2ccc(-c3nn[nH]n3)cc2)CC1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-4-fluorobiphenyl-3-carboxylic acid (300 mg, 0.22 mmol, 50% pure) was initially charged in 4.0 ml of dimethylformamide, and tert-butyl 4-aminopiperidine-1-carboxylate (88 mg, 0.44 mmol) was then added. N,N-Diisopropylethylamine (0.15 ml, 0.88 mmol) and 1-[bis(dimethylamino)methylene]-1H-1,2,3-triazolo[4,5-b]pyridinium 3-oxide hexafluorophosphate (125 mg, 0.33 mmol) were then added, and the mixture was stirred at RT overnight. The reaction mixture was diluted with acetonitrile and purified by preparative HPLC (mobile phase: acetonitrile/water with 0.1% formic acid (gradient)). This gave 112 mg (56% of theory) of the title compound.

LC-MS (Method 1): Rₜ=1.17 min; MS (ESIpos): m/z=868 [M+H]⁺.

¹H-NMR (400 MHz, DMSO-d₆): δ=ppm 16.64 (br. s., 1H), 10.45 (s, 1H), 8.34 (d, 1H), 8.15 (d, 1H), 7.99 (d, 2H), 7.87-7.72 (m, 4H), 7.61 (d, 2H), 7.41 (d, 2H), 7.34 (t, 1H), 6.80-6.70 (m, 1H), 4.76-4.65 (m, 1H), 4.02-3.83 (m, 3H), 3.14-3.05 (m, 1H), 2.98-2.81 (m, 3H), 2.78-2.70 (m, 2H), 2.17-2.06 (m, 1H), 1.86-1.76 (m, 2H), 1.75-1.53 (m, 4H), 1.40 (s, 8H), 1.36 (s, 9H), 1.31-1.09 (m, 3H), 0.91-0.74 (m, 2H).

### Example 56A

### tert-Butyl [(trans-4-{[(2S)-3-{2′-methyl-5′-[(2-oxopiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]carbamate

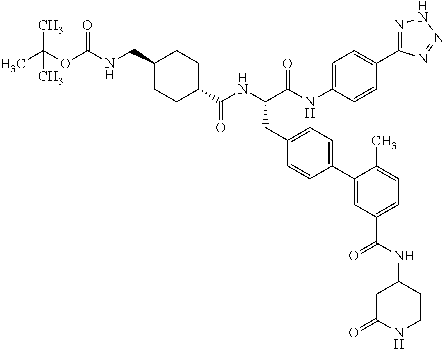 [SMILES: Cc1ccc(C(=O)NC2CCNC(=O)C2)cc1-c1ccc(C[C@H](NC(=O)[C@H]2CC[C@H](CNC(=O)OC(C)(C)C)CC2)C(=O)Nc2ccc(-c3nn[nH]n3)cc2)cc1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-6-methylbiphenyl-3-carboxylic acid (120 mg, 0.15 mmol, 88% pure) from Example 12A was initially charged in 3.0 ml of dimethylformamide, and 4-aminopiperidin-2-one (44 mg, 0.39 mmol) was then added. N,N-Diisopropylethylamine (0.08 ml, 0.46 mmol) and 1-[bis(dimethylamino)methylene]-1H-1,2,3-triazolo[4,5-b]pyridinium 3-oxide hexafluorophosphate (88 mg, 0.23 mmol) were then added, and the mixture was stirred at RT overnight. The reaction mixture was diluted with water/acetonitrile and filtered, and the filtrate was purified by preparative HPLC (mobile phase: acetonitrile/water with 0.1% formic acid (gradient)). This gave 81 mg (68% of theory) of the title compound.

LC-MS (Method 1): Rₜ=0.91 min; MS (ESIpos): m/z=778 [M+H]⁺.

¹H-NMR (400 MHz, DMSO-d₆): δ=ppm 16.73 (br. s., 1H), 10.45 (s, 1H), 8.41 (d, 1H), 8.20 (d, 1H), 7.99 (d, 2H), 7.82 (d, 2H), 7.77-7.71 (m, 1H), 7.70-7.66 (m, 1H), 7.59-7.52 (m, 1H), 7.45-7.32 (m, 3H), 7.29 (d, 2H), 6.84-6.73 (m, 1H), 4.79-4.68 (m, 1H), 4.25-4.11 (m, 1H), 3.24-3.06 (m, 3H), 3.00-2.88 (m, 1H), 2.79-2.70 (m, 2H), 2.48-2.40 (m, 1H), 2.29-2.22 (m, 1H), 2.18-2.07 (m, 1H), 1.96-1.87 (m, 1H), 1.75-1.50 (m, 5H), 1.37 (s, 9H), 1.31-1.08 (m, 3H), 0.91-0.75 (m, 2H).

### Example 57A

### tert-Butyl [(trans-4-{[(2S)-3-(3′-{[4-(dimethylamino)cyclohexyl]carbamoyl}-5′-methylbiphenyl-4-yl)-1-oxo-1-{[4-(2H-tetrazol-5-yl)phenyl]amino}propan-2-yl]carbamoyl}cyclohexyl)methyl]carbamate

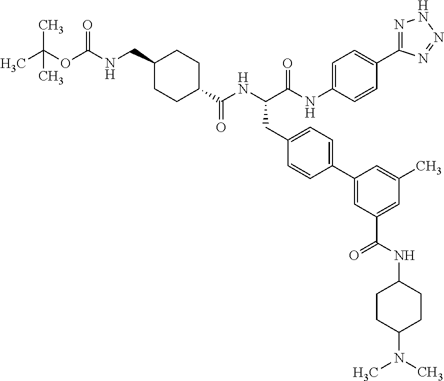 [SMILES: Cc1cc(C(=O)NC2CCC(N(C)C)CC2)cc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5-methylbiphenyl-3-carboxylic acid (100 mg, 0.15 mmol) was initially charged in 2.0 ml of dimethylformamide, and N,N-dimethylcyclohexane-1,4-diamine (42 mg, 0.29 mmol) was then added. N,N-Diisopropylethylamine (0.10 ml, 0.59 mmol) and 1-[bis(dimethylamino)methylene]-1H-1,2,3-triazolo[4,5-b]pyridinium 3-oxide be xafluorophosphate (84 mg, 0.22 mmol) were then added, and the mixture was allowed to stand at RT overnight. The reaction mixture was diluted with acetonitrile and purified by preparative HPLC (mobile phase: acetonitrile/water with 0.1% formic acid (gradient)). This gave 38 mg (30% of theory) of the title compound.

LC-MS (Method 1): Rₜ=0.83 min; MS (ESIpos): m/z=806 [M+H]⁺.

¹H-NMR (400 MHz, DMSO-d₆): δ=ppm 10.16 (s, 1H), 8.34-8.28 (m, 1H), 8.19-8.06 (m, 2H), 7.91 (d, 2H), 7.83 (d, 1H), 7.67-7.56 (m, 6H), 7.40 (d, 2H), 6.79-6.72 (m, 1H), 4.76-4.66 (m, 1H), 4.07-3.73 (m, 1H), 3.50-3.30 (m, 2H), 3.15-3.06 (m, 2H), 2.99-2.88 (m, 1H), 2.78-2.72 (m, 2H), 2.68 (s, 3H), 2.58 (s, 2H), 2.41 (d, 3H), 2.19-2.09 (m, 1H), 2.04-1.89 (m, 3H), 1.83-1.43 (m, 8H), 1.36 (s, 9H), 1.29-1.12 (m, 3H), 0.91-0.75 (m, 2H).

### Example 58A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5-methylbiphenyl-3-carboxylic acid

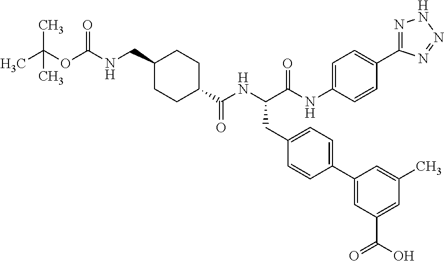 [SMILES: Cc1cc(C(=O)O)cc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1]

3-(Dihydroxyboryl)-5-methylbenzoic acid (402 mg, 2.23 mmol), 4-bromo-N-alpha-[(trans-4-{[(left-butoxycarbonyl)amino]methyl}cyclohexyl)carbonyl]-N-[4-(2H-tetrazol-5-yl)phenyl]-L-phenylalaninamide (1.00 g, 1.60 mmol) from Example 6A and 507 mg (4.79 mmol) of sodium carbonate were initially charged in a mixture of 10.0 ml of DMF and 1.67 ml of water under argon. Subsequently, 1,1-bis(diphenylphosphino)ferrocenedichloropalladium(II) (117 mg, 0.16 mmol) was added and the mixture was stirred at 140° C. in a microwave for 1 h. Thereafter, the reaction mixture was diluted with 50 ml of water, acidified with 1M hydrochloric acid and extracted twice with 50 ml each time of ethyl acetate. The combined organic phases were washed once each with water and saturated aqueous sodium chloride solution, dried over magnesium sulphate, filtered and concentrated. The solid which had formed was filtered off, washed with ethyl acetate and dried under high vacuum. This gave 268 mg of the title compound as a crude product, which was converted further without further purification.

LC-MS (Method 1): Rₜ=1.00 min; MS (ESIpos): m/z=682 [M+H]⁺.

¹H-NMR (400 MHz, DMSO-d₆): δ=ppm 10.44 (s, 1H), 8.14 (d, 1H), 7.99 (d, 2H), 7.82 (d, 2H), 7.64-7.56 (m, 3H), 7.49-7.43 (m, 1H), 7.38 (d, 2H), 7.34 (d, 1H), 6.79-6.71 (m, 1H), 4.74-4.66 (m, 1H), 3.75-3.59 (m, 4H), 3.58-3.40 (m, 2H), 3.23-3.12 (m, 2H), 3.12-3.05 (m, 1H), 2.97-2.88 (m, 1H), 2.79-2.70 (m, 2H), 2.25 (s, 3H), 2.17-2.08 (m, 1H), 1.74-1.55 (m, 4H), 1.36 (s, 9H), 1.30-1.11 (m, 4H), 0.89-0.77 (m, 2H).

### Example 59A

### tert-Butyl [(trans-4-{[(2S)-3-[4′-methyl-3′-(morpholin-4-ylcarbonyl)biphenyl-4-yl]-1-oxo-1-{[4-(2H-tetrazol-5-yl)phenyl]amino}propan-2-yl]carbamoyl}cyclohexyl)methyl]carbamate

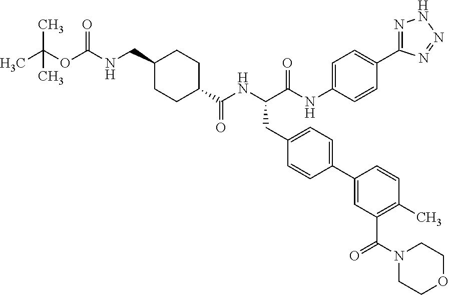 [SMILES: Cc1ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1C(=O)N1CCOCC1]

4′-[(2S)-2-{[(trans-4-{[(tert-Butoxycarbonyl)amino]methyl}cyclohexyl) carbonyl]amino}-3-oxo-3-{[4-(2H-tetrazol-5-yl)phenyl]amino}propyl]-4-methylbiphenyl-3-carboxylic acid from Example 12A (100 mg, 0.15 mmol) and morpholine (26 mg, 0.29 mmol) were initially charged in 3.0 ml of dimethylformamide, N,N-diisopropylethylamine (0.08 ml, 0.44 mmol) and 1-[bis(dimethylamino)methylene]-1H-1,2,3-triazolo[4,5-b]pyridinium 3-oxide hexafluorophosphate (84 mg, 0.22 mmol) were added and the mixture was stirred at RT overnight. The reaction mixture was diluted with acetonitrile/water and filtered, and the filtrate was purified by preparative HPLC (mobile phase: acetonitrile/water with 0.1% formic acid (gradient)). This gave 81 mg (73% of theory) of the title compound.

LC-MS (Method 1): Rₜ=0.99 min; MS (ESIpos): m/z=751 [M+H]⁺.

### Example 60A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-4-methylbiphenyl-3-carboxylate

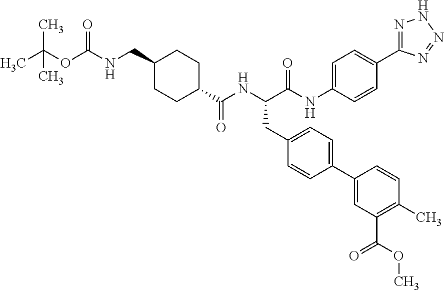 [SMILES: COC(=O)c1cc(-c2ccc(C[C@H](NC(=O)[C@H]3CC[C@H](CNC(=O)OC(C)(C)C)CC3)C(=O)Nc3ccc(-c4nn[nH]n4)cc3)cc2)ccc1C]

Under argon, 4-bromo-N-alpha-[(trans-4-{[(tert-butoxycarbonyl)amino]methyl}cyclohexyl) carbonyl]-N-[4-(2H-tetrazol-5-yl)phenyl]-L-phenylalaninamide (1.00 g, 1.60 mmol) from Example 6A, [3-(methoxycarbonyl)-4-methylphenyl]boronic acid (433 mg, 2.23 mmol) and sodium carbonate (507 mg, 4.79 mmol) were initially charged in 12 ml of DMF/water (10:2). Subsequently, 1, 1-bis(diphenylphosphino)ferrocenedichloropalladium(II) (117 mg, 0.16 mmol) was added and the mixture was stirred at 140° C. in a microwave for 1 h. At RT, the reaction mixture was then diluted with 50 ml of water, acidified with 1M hydrochloric acid and extracted twice with 70 ml each time of ethyl acetate. The combined organic phases were washed once each with water and saturated aqueous sodium chloride solution, dried over sodium sulphate and filtered, and the filtrate was concentrated. The residue obtained was taken up in ethyl acetate, and the precipitate formed was filtered off, washed with ethyl acetate and dried under high vacuum. The crude product obtained in this manner (82% pure) was reacted further without further purification.

LC-MS (Method 1): Rₜ=1.14 min; MS (ESIpos): m/z=696 [M+H]⁺.

### Example 61A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-4-methylbiphenyl-3-carboxylic acid

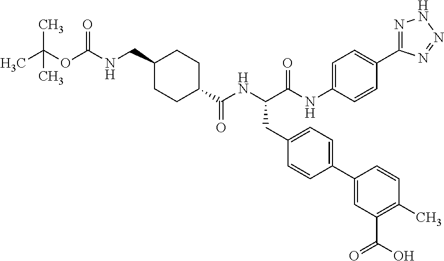 [SMILES: Cc1ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1C(=O)O]

Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-4-methylbiphenyl-3-carboxylate (608 mg, 0.72 mmol, 82% pure) was initially charged in 6.0 ml of THF, and lithium hydroxide (171 mg, 7.15 mmol) and 2.0 ml of water were then added. The mixture was stirred at RT for 2 days and at 45° C. for 6 h. The reaction mixture was then acidified with 1M hydrochloric acid, and the solid formed was filtered off, washed with water and dried under high vacuum. This gave 549 mg (84% pure) of the title compound which was reacted further without further purification.

LC-MS (Method 1): Rₜ=1.01 min; MS (ESIpos): m/z=682 [M+H]⁺.

### Example 62A

### tert-Butyl [(trans-4-{[(2S)-3-[5′-(dimethylcarbamoyl)-2′,4′-dimethylbiphenyl-4-yl]-1-oxo-1-{[4-(2H-tetrazol-5-yl)phenyl]amino}propan-2-yl]carbamoyl}cyclohexyl)methyl]carbamate

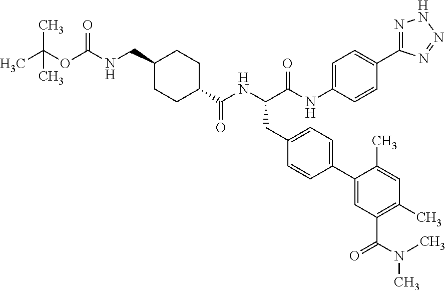 [SMILES: Cc1cc(C)c(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1C(=O)N(C)C]

4′-[(2S)-2-{[(trans-4-{[(tert-Butoxycarbonyl)amino]methyl}cyclohexyl)carbonyl]amino}-3-oxo-3-[4-(2H-tetrazol-5-yl)phenyl]amino 1 propyl]-4,6-dimethylbiphenyl-3-carboxylic acid (95 mg, 0.14 mmol) was initially charged in 2.0 ml of dimethylformamide, and N,N-dimethylamine (40% in water, 31 mg, 0.27 mmol) was then added. N,N-Diisopropylethylamine (0.10 ml, 0.55 mmol) and 1-[bis(dimethylamino)methylene]-1H-1,2,3-triazolo[4,5-b]pyridinium 3-oxide hexafluorophosphate (78 mg, 0.21 mmol) were then added, and the mixture was stirred at RT for 1 h and allowed to stand overnight. The reaction mixture was diluted with acetonitrile and purified by preparative HPLC (mobile phase: acetonitrile/water with 0.1% formic acid (gradient)). This gave 80 mg (78% of theory) of the title compound.

¹H-NMR (400 MHz, DMSO-d₆): δ=ppm 10.40 (s, 1H), 8.14 (d, 1H), 7.98 (d, 2H), 7.81 (d, 2H), 7.35 (d, 2H), 7.23 (d, 2H), 7.16 (s, 1H), 6.89 (s, 1H), 6.78-6.72 (m, 1H), 4.78-4.69 (m, 1H), 3.14-3.06 (m, 1H), 2.98 (s, 2H), 2.97-2.90 (m, 2H), 2.78 (s, 3H), 2.77-2.72 (m, 2H), 2.18 (s, 3H), 2.14-2.06 (m, 1H), 1.73-1.59 (m, 3H), 1.58-1.51 (m, 1H), 1.37 (s, 9H), 1.30-1.19 (m, 2H), 1.19-1.08 (m, 1H), 0.89-0.75 (m, 2H).

LC-MS (Method 1): Rₜ=1.05 min; MS (ESIpos): m/z=723 [M+H]⁺.

### Example 63A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-4,6-dimethylbiphenyl-3-carboxylic acid

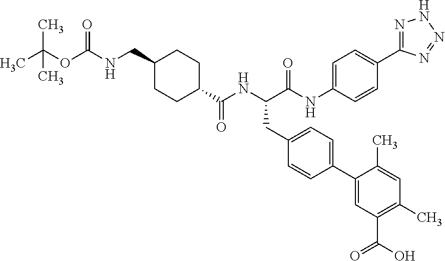 [SMILES: Cc1cc(C)c(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1C(=O)O]

Methyl 4′-[(2S)-2-{[(trans-4-{[(tert-butoxycarbonyl)amino]methyl}cyclohexyl)carbonyl]amino}-3-oxo-3-[4-(2H-tetrazol-5-yl)phenyl]amino 1 propyl]-4,6-dimethylbiphenyl-3-carboxylate (323 mg, 0.46 mmol) was initially charged in 2.4 ml of THF, and a solution of lithium hydroxide (109 mg, 4.55 mmol) in 1.5 ml of water was added. The mixture was allowed to stand at RT overnight and stirred at 60° C. for 90 min A further 10 eq. of lithium hydroxide were then added, and the mixture was stirred at 60° C. for 10 h. A further 10 eq. of lithium hydroxide were then added, and the mixture was stirred at 60° C. for 2 h. The resulting suspension was diluted with water and acidified with 1M hydrochloric acid. The solid present was filtered off, washed with ethyl acetate and dried under high vacuum. This gave 204 mg (60% of theory) of the title compound.

¹H-NMR (400 MHz, DMSO-d₆): δ=ppm 12.70 (br. s., 1H), 10.44 (s, 1H), 8.17 (d, 1H), 7.99 (d, 2H), 7.81 (d, 2H), 7.63 (s, 1H), 7.37 (d, 2H), 7.27-7.19 (m, 3H), 6.82-6.74 (m, 1H), 4.80-4.69 (m, 1H), 3.16-3.06 (m, 1H), 2.98-2.88 (m, 1H), 2.78-2.71 (m, 2H), 2.54 (s, 3H), 2.18 (s, 3H), 2.16-2.06 (m, 1H), 1.73-1.58 (m, 3H), 1.58-1.48 (m, 1H), 1.37 (s, 9H), 1.31-1.06 (m, 3H), 0.89-0.73 (m, 2H).

LC-MS (Method 1): Rₜ=1.04 min; MS (ESIpos): m/z=696 [M+H]⁺.

### Example 64A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-4,6-dimethylbiphenyl-3-carboxylate

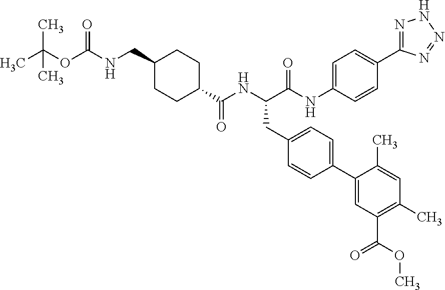 [SMILES: COC(=O)c1cc(-c2ccc(C[C@H](NC(=O)[C@H]3CC[C@H](CNC(=O)OC(C)(C)C)CC3)C(=O)Nc3ccc(-c4nn[nH]n4)cc3)cc2)c(C)cc1C]

Under argon, 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (1.00 g, 1.60 mmol) from Example 6A, methyl 2,4-dimethyl-5-(4,4,5,5-tetramethyl-1,3,2-dioxaborolan-2-yl)benzoate (1.39 g, 2.39 mmol, 50% pure, synthesis described in WO2014/008197) and sodium carbonate (507 mg, 4.79 mmol) were initially charged in 12 ml of DMF/water (10:2). Subsequently, 1,1-bis(diphenylphosphino)ferrocenedichloropalladium(II) (117 mg, 0.16 mmol) was added and the mixture was stirred at 100° C. in a microwave for 1 h. At RT, the reaction mixture was then diluted with 50 ml of water, acidified with 1M hydrochloric acid and extracted twice with 50 ml each time of ethyl acetate. The combined organic phases were washed once each with water and saturated aqueous sodium chloride solution, dried over magnesium sulphate and filtered, and the filtrate was concentrated. The residue obtained was purified by preparative HPLC (mobile phase: acetonitrile/water with 0.1% formic acid (gradient)). This gave 331 mg (28% of theory) of the title compound.

¹H-NMR (400 MHz, DMSO-d₆): δ=ppm 10.40 (s, 1H), 8.14 (d, 1H), 7.99 (d, 2H), 7.81 (d, 2H), 7.62 (s, 1H), 7.37 (d, 2H), 7.26-7.21 (m, 3H), 6.80-6.72 (m, 1H), 4.79-4.69 (m, 1H), 3.79 (s, 3H), 3.16-3.07 (m, 1H), 2.98-2.88 (m, 1H), 2.78-2.72 (m, 2H), 2.54 (s, 3H), 2.19 (s, 3H), 2.16-2.07 (m, 1H), 1.73-1.58 (m, 3H), 1.57-1.49 (m, 1H), 1.34 (br. s., 1H), 1.31-1.18 (m, 2H), 1.18-1.04 (m, 1H), 0.90-0.74 (m, 2H)

LC-MS (Method 1): Rₜ=1.16 min; MS (ESIpos): m/z=696 [M+H]⁺.

### Example 65A

### tert-Butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-5-methylbiphenyl-3-yl}carbonyl)amino]piperidine-1-carboxylate

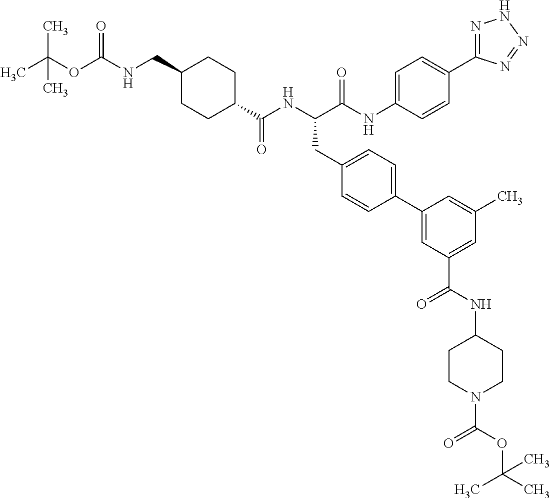 [SMILES: Cc1cc(C(=O)NC2CCN(C(=O)OC(C)(C)C)CC2)cc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-5-methylbiphenyl-3-carboxylic acid (100 mg, 0.15 mmol) was initially charged in 2.0 ml of dimethylformamide, and tert-butyl 4-aminopiperidine-1-carboxylate (59 mg, 0.29 mmol) was then added. N,N-Diisopropylethylamine (0.10 ml, 0.59 mmol) and 1-[bis(dimethylamino)methylene]-1H-1,2,3-triazolo[4,5-b]pyridinium 3-oxide hexafluorophosphate (84 mg, 0.22 mmol) were then added, and the mixture was stirred at RT for 6 h and allowed to stand at RT for 2 days. The reaction mixture was diluted with acetonitrile and purified by preparative HPLC (mobile phase: acetonitrile/water with 0.1% formic acid (gradient)). This gave 66 mg (49% of theory) of the title compound.

¹H-NMR (400 MHz, DMSO-d₆): δ=ppm 16.71 (br. s., 1H), 10.47 (s, 1H), 8.32 (d, 1H), 8.18 (d, 1H), 7.99 (d, 2H), 7.89-7.78 (m, 3H), 7.69-7.57 (m, 4H), 7.42 (d, 2H), 6.81-6.71 (m, 1H), 4.76-4.66 (m, 1H), 4.06-3.86 (m, 3H), 3.16-3.04 (m, 1H), 2.99-2.69 (m, 5H), 2.41 (s, 3H), 2.18-2.06 (m, 1H), 1.84-1.75 (m, 2H), 1.74-1.52 (m, 4H), 1.41 (s, 9H), 1.36 (s, 9H), 1.30-1.10 (m, 3H), 0.91-0.74 (m, 2H).

LC-MS (Method 1): Rₜ=1.18 min; MS (ESIpos): m/z=864 [M+H]⁺.

### Example 66A

### 3-{5-[4-({(2S)-2-{[(trans-4-{[(tert-Butoxycarbonyl)amino]methyl}cyclohexyl)carbonyl]amino}-3-[2′-methyl-5′-(methylcarbamoyl)biphenyl-4-yl]propanoyl}amino)phenyl]-4H-1,2,4-triazol-3-yl}-2,2,3,3-tetrafluoropropanoic acid

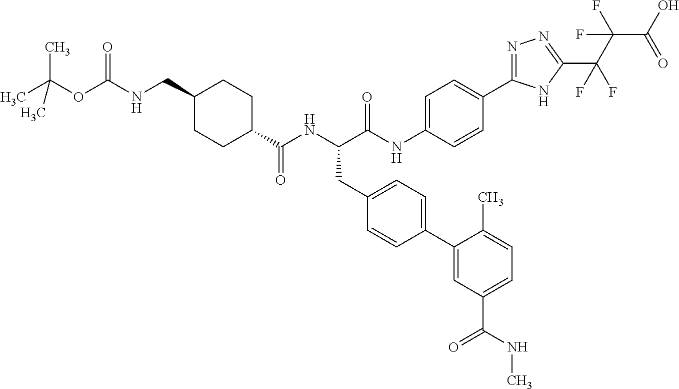 [SMILES: CNC(=O)c1ccc(C)c(-c2ccc(C[C@H](NC(=O)[C@H]3CC[C@H](CNC(=O)OC(C)(C)C)CC3)C(=O)Nc3ccc(-c4nnc(C(F)(F)C(F)(F)C(=O)O)[nH]4)cc3)cc2)c1]

3-{5-[4-({(2S)-2-{[(trans-4-{[(tert-Butoxycarbonyl)amino]methyl}cyclohexyl)carbonyl]amino}-3-[2′-methyl-5′-(methylcarbamoyl)biphenyl-4-yl]propanoyl}amino)phenyl]-4H-1,2,4-triazol-3-yl}-2,2,3,3-tetrafluoropropanoic acid (70 mg, 0.09 mmol) and methylamine (0.08 ml, 0.17 mmol, 2M in THF) were initially charged in 1.0 ml of dimethylformamide. N,N-Diisopropylethylamine (0.04 ml, 0.26 mmol) and 1-[bis(dimethylamino)methylene]-1H-1,2,3-triazolo[4,5-b]pyridinium 3-oxide hexafluorophosphate (48 mg, 0.13 mmol) were then added, and the mixture was shaken at RT overnight. The reaction mixture was diluted with acetonitrile and purified by preparative HPLC (mobile phase: acetonitrile/water with 0.1% formic acid (gradient)). This gave 6 g of crude product (90% pure), which was reacted further without further purification.

LC-MS (Method 1): Rₜ=0.95 min; MS (ESIpos): m/z=838 [M+H-HCl]⁺.

### Example 67A

### (2S)-2-{[(trans-4-{[(tert-Butoxycarbonyl)amino]methyl}cyclohexyl)carbonyl]amino}-3-[5′-(methoxycarbonyl)-2′-methylbiphenyl-4-yl]propanoic acid

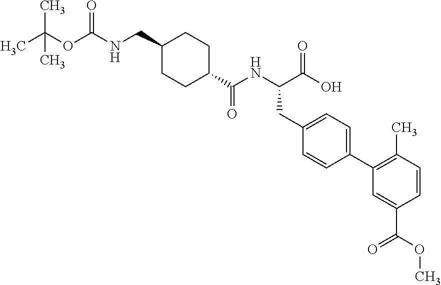 [SMILES: COC(=O)c1ccc(C)c(-c2ccc(C[C@H](NC(=O)[C@H]3CC[C@H](CNC(=O)OC(C)(C)C)CC3)C(=O)O)cc2)c1]

4-Bromo-N-[(trans-4-{[(tert-butoxycarbonyl)amino]methyl}cyclohexyl)carbonyl]-L-phenylalanine (43.4 g, 90 mmol) and methyl 4-methyl-3-(4,4,5,5-tetramethyl-1,3,2-dioxaborolan-2-yl)benzoate (33.6 g, 117 mmol) were dissolved in DMF (400 ml), and an aqueous 2N sodium carbonate solution (135 ml) was added. The reaction solution was degassed with argon for 5 min, after which the catalyst 1,1′-bis(diphenylphosphino)ferrocenepalladium(II) chloride (6.6 g, 9.0 mmol) was added. The mixture was stirred at 120° C. (oil bath temperature) for 30 min. The contents of the flask were filtered through kieselguhr and washed through with ethyl acetate. The solvent was removed on a rotary evaporator and the residue was partitioned between ethyl acetate and 10% strength aqueous citric acid solution. The mixture was extracted with ethyl acetate, the combined organic phases were washed with saturated aqueous sodium chloride solution and dried over magnesium sulphate, and the solvent was removed on a rotary evaporator. The residue was suspended in diethyl ether and the solid was filtered off with suction, washed with diethyl ether and dried under high vacuum. This gave 38.1 g (77% of theory) of the title compound.

¹H-NMR (400 MHz, DMSO-d₆): δ=ppm 0.71-0.89 (m, 2H), 1.03-1.29 (m, 3H), 1.36 (s, 9H), 1.45-1.54 (m, 1H), 1.57-1.73 (m, 3H), 1.98-2.10 (m, 1H), 2.27 (s, 3H), 2.74 (m, 2H), 2.85-2.95 (m, 1H), 3.14 (dd, 1H), 3.84 (s, 3H), 4.50 (br. s., 1H), 6.71-6.82 (m, 1H), 7.23-7.35 (m, 4H), 7.45 (d, 1H), 7.71 (d, 1H), 7.85 (dd, 1H), 8.03 (d, 1H), 12.68 (br. s, 1H).

LC-MS (Method 1): Rₜ=1.13 min; MS (ESIpos): m/z=553 [M+H]⁺.

### Example 68A

### 3-{5-[4-({(2S)-2-{[(trans-4-{[(tert-Butoxycarbonyl)amino]methyl}cyclohexyl)carbonyl]amino}-3-[5′-(methoxycarbonyl)-2′-methylbiphenyl-4-yl]propanoyl}amino)phenyl]-1H-1,2,4-triazol-3-yl}-2,2,3,3-tetrafluoropropanoic acid

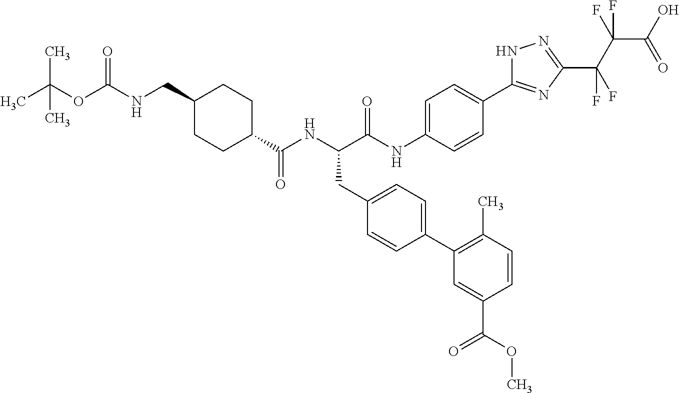 [SMILES: COC(=O)c1ccc(C)c(-c2ccc(C[C@H](NC(=O)[C@H]3CC[C@H](CNC(=O)OC(C)(C)C)CC3)C(=O)Nc3ccc(-c4nc(C(F)(F)C(F)(F)C(=O)O)n[nH]4)cc3)cc2)c1]

N,N-Diisopropylamine (0.79 ml, 4.5 mmol) was added to a solution of (2S)-2-{[(trans-4-{[(tert-butoxycarbonyl)amino]methyl}cyclohexyl)carbonyl]amino}-3-[5′-(methoxycarbonyl)-2′-methylbiphenyl-4-yl]propanoic acid (500 mg, 0.91 mmol) and 3-[5-(4-aminophenyl)-1H-1,2,4-triazol-3-yl]-2,2,3,3-tetrafluoropropanoic acid (682 mg, 1.81 mmol) in DMF (7.4 ml), and HATU (516 mg, 1.36 mmol) was added. The reaction mixture was stirred at RT overnight (about 16 h), and the solvent was then removed under high vacuum. The residue was dissolved in DMSO, a little water and acetonitrile was added and the solution was filtered through a Millipore filter and then purified by preparative HPLC (mobile phase: gradient of acetonitrile/water with 0.1% trifluoroacetic acid). This gave 466 mg (25% of theory) of the title compound.

LC-MS (Method 1): Rₜ=1.09 min; MS (ESIpos): m/z=839 [M+H]⁺.

### Example 69A

### 4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxopropyl]-6-methylbiphenyl-3-carboxylic acid

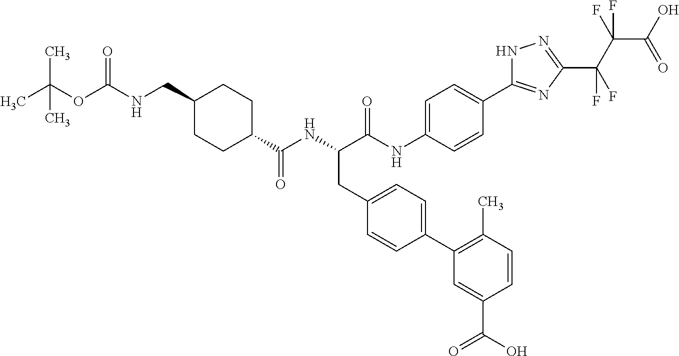 [SMILES: Cc1ccc(C(=O)O)cc1-c1ccc(C[C@H](NC(=O)[C@H]2CC[C@H](CNC(=O)OC(C)(C)C)CC2)C(=O)Nc2ccc(-c3nc(C(F)(F)C(F)(F)C(=O)O)n[nH]3)cc2)cc1]

3-{5-[4-({(2S)-2-{[(trans-4-{[(tert-Butoxycarbonyl)amino]methyl}cyclohexyl)carbonyl]-amino}-3-[5′-(methoxycarbonyl)-2′-methylbiphenyl-4-yl]propanoyl}amino)phenyl]-1H-1,2,4-triazol-3-yl}-2,2,3,3-tetrafluoropropanoic acid (463 mg, 0.49 mmol) was dissolved in tetrahydrofuran/water 3:1 (8 ml), lithium hydroxide monohydrate (62 mg, 1.48 mmol) was added and the mixture was stirred at RT overnight. The mixture was added to ethyl acetate/water and acidified slightly (pH 4-5) with 0.5N hydrochloric acid. The mixture was extracted three times with ethyl acetate. The combined organic phases were washed with saturated aqueous sodium chloride solution, dried over sodium sulphate and filtered, and the solvent was removed under reduced pressure. The residue was dissolved in DMSO, a little water and acetonitrile was added and the solution was filtered through a Millipore filter and then purified by preparative HPLC (mobile phase: gradient of acetonitrile/water with 0.1% trifluoroacetic acid). This gave 423 mg (95% of theory) of the title compound.

LC-MS (Method 1): Rt=1.00 min; MS (ESIpos): m/z=825 [M+H]⁺.

### Example 70A

### 3-[5-(4-{[(2S)-2-{[(trans-4-{[(tert-Butoxycarbonyl)amino]methyl}cyclohexyl)carbonyl]amino}-3-{2′-methyl-5′-[(1-methylpiperidin-4-yl)carbamoyl]biphenyl-4-yl}propanoyl]amino}phenyl)-1H-1,2,4-triazol-3-yl]-2,2,3,3-tetrafluoropropanoic acid

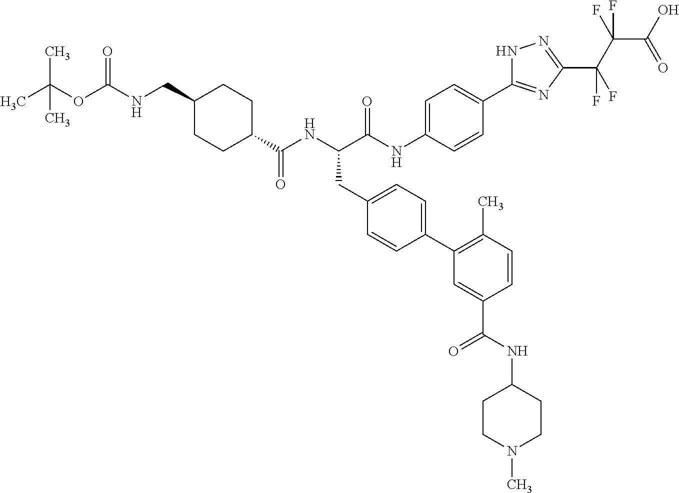 [SMILES: Cc1ccc(C(=O)NC2CCN(C)CC2)cc1-c1ccc(C[C@H](NC(=O)[C@H]2CC[C@H](CNC(=O)OC(C)(C)C)CC2)C(=O)Nc2ccc(-c3nc(C(F)(F)C(F)(F)C(=O)O)n[nH]3)cc2)cc1]

N,N-Diisopropylamine (0.14 ml, 0.80 mmol) was added to a solution of 4′-[(2S)-2-{[(trans-4-{[(tert-butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxopropyl]-6-methylbiphenyl-3-carboxylic acid (220 mg, 0.27 mmol) and 1-methylpiperidine-4-amine (61 mg, 0.53 mmol) in DMF (2.75 ml), and HATU (152 mg, 0.40 mmol) was added. The reaction mixture was stirred at RT overnight. The mixture was diluted with water/acetonitrile and filtered through a Millipore filter, then purified by preparative HPLC (mobile phase: gradient of acetonitrile/water with 0.1% trifluoroacetic acid). This gave 87 mg (29% of theory) of the title compound.

LC-MS (Method 1): Rₜ=0.86 min; MS (ESIpos): m/z=921 [M+H]⁺.

### Example 71A

### N-Isopropyl-4-methyl-3-(4,4,5,5-tetramethyl-1,3,2-dioxaborolan-2-yl)benzamide

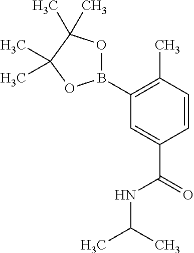 [SMILES: Cc1ccc(C(=O)NC(C)C)cc1B1OC(C)(C)C(C)(C)O1]

Under argon, N,N-diisopropylamine (1.7 ml, 9.78 mmol) was added to a solution of 4-methyl-3-(4,4,5,5-tetramethyl-1,3,2-dioxaborolan-2-yl)benzoic acid (1.17 g, 4.45 mmol) and isopropylamine (0.38 ml, 4.45 mmol) in DMF (45 ml), and a solution of HATU (2.03 g, 5.33 mmol) in DMF (15 ml) was added. The reaction mixture was stirred at RT with LC/MS monitoring until the reaction had gone to completion. The solvent was removed on a rotary evaporator, water was added to the residue and the solid was then filtered off with suction. This gave 1.2 g (83% of theory) of the title compound.

LC-MS (Method 1): Rₜ=1.14 min; MS (ESIpos): m/z=304 [M+H]⁺.

### Example 72A

### (2S)-2-{[(trans-4-{[(tert-Butoxycarbonyl)amino]methyl}cyclohexyl)carbonyl]amino}-3-[5′-(isopropylcarbamoyl)-2′-methylbiphenyl-4-yl]propanoic acid

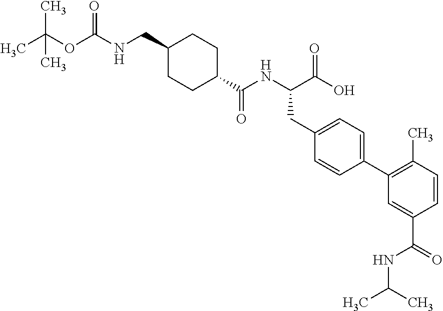 [SMILES: Cc1ccc(C(=O)NC(C)C)cc1-c1ccc(C[C@H](NC(=O)[C@H]2CC[C@H](CNC(=O)OC(C)(C)C)CC2)C(=O)O)cc1]

Aqueous 2N sodium carbonate solution (8.7 ml) was added to a solution of 4-bromo-N-[(trans-4-{[(tert-butoxycarbonyl)amino]methyl}cyclohexyl)carbonyl]-L-phenylalanine (1.64 g, 3.39 mmol) and N-isopropyl-4-methyl-3-(4,4,5,5-tetramethyl-1,3,2-dioxaborolan-2-yl)benzamide (1.33 g, 4.07 mmol) in DMF (34 ml). The solution was degassed with argon for 25 min, after which the catalyst 1,1′-bis(diphenylphosphino)ferrocenepalladium(II) chloride dichloromethane complex (277 mg, 0.34 mmol) was added. The reaction mixture was stirred at 120° C. for 30 min. The mixture was filtered through kieselguhr and the residue was washed with ethyl acetate. The solvent was removed on a rotary evaporator and the residue was dried under high vacuum. The residue was dissolved in ethyl acetate, washed once with 10% strength aqueous citric acid solution and once with saturated aqueous sodium chloride solution and then dried with magnesium sulphate. The suspension was filtered and the solvent was removed on a rotary evaporator. The residue was purified by flash chromatography (mobile phase: dichloromethane/methanol, gradient 10:0 to 10:0.5). This gave 1.72 g (81% of theory) of the title compound.

¹H-NMR (400 MHz, DMSO-d₆): δ=ppm 0.65-0.93 (m, 2H), 1.14 (d, 7H), 1.19-1.31 (m, 2H), 1.36 (s, 9H), 1.48-1.56 (m, 1H), 1.58-1.73 (m, 3H), 2.00-2.11 (m, 1H), 2.24 (s, 3H), 2.70-2.76 (m, 3H), 2.90-2.93 (m, 1H), 3.07-3.19 (m, 1H), 4.04-4.15 (m, 1H), 4.42-4.52 (m, 1H), 6.71-6.83 (m, 1H), 7.31 (m, 5H), 7.67 (d, 1H), 7.71-7.78 (m, 1H), 8.04 (d, 1H), 8.20 (d, 1H), 12.68 (br. s, 1H)

LC-MS (Method 1): Rₜ=1.04 min; MS (ESIpos): m/z=580 [M+H]⁺.

### Example 73A

### tert-Butyl 4-{2-[(4-nitrophenyl)carbonoimidoyl]hydrazino}-4-oxobutanoate

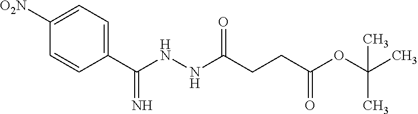 [SMILES: CC(C)(C)OC(=O)CCC(=O)NNC(=N)c1ccc([N+](=O)[O-])cc1]

Under argon, HATU (2.03 g, 5.33 mmol) was added to a solution of 4-nitrobenzenecarboximidohydrazide (1.72 g, 9.57 mmol) and 4-tert-butoxy-4-oxobutanoic acid (1.67 g, 9.57 mmol) in THF (35 ml), and the mixture was stirred at RT overnight. The solvent was removed on a rotary evaporator and the residue was dried under high vacuum. The residue was dissolved in a little methanol and filtered through a Millipore filter, then purified by preparative HPLC (mobile phase: gradient of acetonitrile/water with 0.1% trifluoroacetic acid). This gave 1.47 g (35% of theory) of the title compound.

LC-MS (Method 1): Rₜ=0.62 min; MS (ESIpos): m/z=337 [M+H]⁺.

### Example 74A

### tert-Butyl 3-[5-(4-nitrophenyl)-1H-1,2,4-triazol-3-yl]propanoate

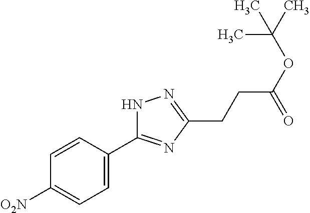 [SMILES: CC(C)(C)OC(=O)CCc1n[nH]c(-c2ccc([N+](=O)[O-])cc2)n1]

tert-Butyl 4{-2-[(4-nitrophenyl)carbonoimidoyl]hydrazino}-4-oxobutanoate (1.46 g, 4.34 mmol) was dissolved in 1-methylpyrrolidine (15 ml) and stirred at 120° C. for 3 days. The solvent was removed on a rotary evaporator and the residue was dried under high vacuum. The substance was recrystallized from methanol and the solid was filtered off with suction, washed with a little methanol and then dried under high vacuum. This gave 657.4 mg (44% of theory) of the title compound.

LC-MS (Method 1): Rt=0.62 min; MS (ESIpos): m/z=319 [M+H]⁺.

### Example 75A

### tert-Butyl 3-[5-(4-aminophenyl)-1H-1,2,4-triazol-3-yl]propanoate

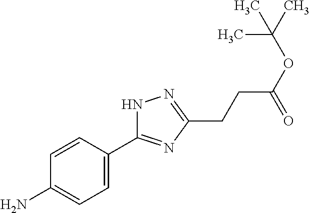 [SMILES: CC(C)(C)OC(=O)CCc1n[nH]c(-c2ccc(N)cc2)n1]

Tin(II) chloride dihydrate was added to a solution of tert-butyl 3-[5-(4-nitrophenyl)-1H-1,2,4-triazol-3-yl]propanoate (622 mg, 1.95 mmol) in ethanol (20 ml), and the reaction mixture was stirred at 70° C. for 1 h. The solution was cooled to RT and then added to ice-water, and the mixture was adjusted to pH 8 with solid sodium carbonate. The mixture was diluted with water and extracted three times with ethyl acetate. The organic phases were washed with saturated aqueous sodium chloride solution, dried over sodium sulphate and then filtered, and the solvent was then removed on a rotary evaporator. The residue was recrystallized from diethyl ether/pentane, filtered off with suction and washed with pentane, and then dried under high vacuum. This gave 537.7 mg (95% of theory) of the title compound.

LC-MS (Method 1): Rₜ=0.65 min; MS (ESIpos): m/z=289 [M+H]⁺.

### Example 76A

### tert-Butyl 3-{5-[4-((2S)-2-{[(trans-4-{[(tert-butoxycarbonyl)amino]methyl}cyclohexyl)-carbonyl]amino}-3-[5′-(isopropylcarbamoyl)-2′-methylbiphenyl-4-yl]propanoyl}amino)phenyl]-1H-1,2,4-triazol-3-yl propanoate

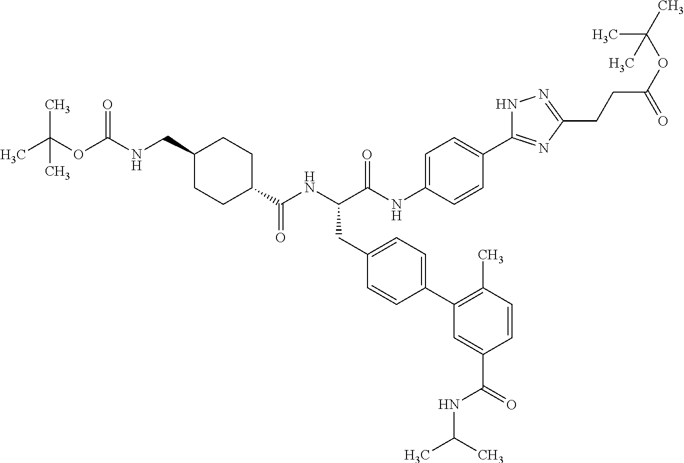 [SMILES: Cc1ccc(C(=O)NC(C)C)cc1-c1ccc(C[C@H](NC(=O)[C@H]2CC[C@H](CNC(=O)OC(C)(C)C)CC2)C(=O)Nc2ccc(-c3nc(CCC(=O)OC(C)(C)C)n[nH]3)cc2)cc1]

N,N-Diisopropylamine (0.9 ml, 0.52 mmol) was added to a solution of (2S)-2-{[(trans-4-{[(tert-butoxycarbonyl)amino]methyl}cyclohexyl)carbonyl]amino}-3-[5′-(isopropylcarbamoyl)-2′-methylbiphenyl-4-yl]propanoic acid (100 mg, 0.17 mmol) and tert-butyl 3-[5-(4-aminophenyl)-1H-1,2,4-triazol-3-yl]propanoate (99 mg, 0.35 mmol) in DMF (1 ml), and HATU (98 mg, 0.26 mmol) was added. The reaction mixture was stirred at RT overnight (about 16 h). The residue was diluted with water/acetonitrile and filtered through a Millipore filter, then purified by preparative HPLC (mobile phase: gradient of acetonitrile/water with 0.1% trifluoroacetic acid). This gave 49.3 mg (34% of theory) of the title compound.

LC-MS (Method 1): Rₜ=1.14 min; MS (ESIpos): m/z=850 [M+H]⁺.

### Example 77A

### Methyl 3-{5-[4-({4-bromo-N-[(trans-4-{[(tert-butoxycarbonyl)amino]methyl}cyclohexyl)carbonyl]-L-phenylalanyl}amino)phenyl]triazol-3-yl}-2,2,3,3-tetrafluoropropanoate

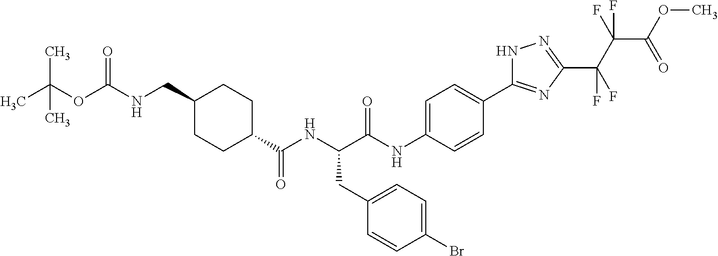 [SMILES: COC(=O)C(F)(F)C(F)(F)c1n[nH]c(-c2ccc(NC(=O)[C@H](Cc3ccc(Br)cc3)NC(=O)[C@H]3CC[C@H](CNC(=O)OC(C)(C)C)CC3)cc2)n1]

4-Bromo-N-[(trans-4-{[(tert-butoxycarbonyl)amino]methyl}cyclohexyl)carbonyl]-L-phenylalanine (1500 mg, 3.1 mmol) and methyl 3-[5-(4-aminophenyl)-1H-1,2,4-triazol-3-yl]-2,2,3,3-tetrafluoropropanoate (1185 mg, 3.7 mmol) were dissolved in 10 ml of pyridine and admixed with 2,4,6-tripropyl-1,3,5,2,4,6-trioxatriphosphinane 2,4,6-trioxide (50% in DMF, 7.2 ml, 12.4 mmol) and stirred at 85° C. for 5 h. Water was added, and the pyridine was removed under reduced pressure. Dilute ammonium chloride solution was added to the residue, and the mixture was extracted three times with ethyl acetate. The combined organic phases were washed with water and saturated aqueous sodium chloride solution, and dried over sodium sulphate and under reduced pressure. The residue was purified by chromatography (silica gel, mobile phase: dichloromethane/methanol=10/1). This gave 1675 mg (63% of theory) of the title compound.

¹H-NMR (400 MHz, DMSO-d₆): δ=ppm 0.82 (m, 2H), 1.05-1.30 (m, 3H), 1.36 (s, 9H), 1.53-1.60 (m, 1H), 1.68 (m, 3H), 2.03-2.14 (m, 1H), 2.70-2.78 (m, 2H), 2.80-2.91 (m, 1H), 2.97-3.09 (m, 1H), 3.95 (s, 2H), 4.57-4.72 (m, 1H), 6.72-6.82 (m, 1H), 7.26 (d, 2H), 7.48 (d, 2H), 7.73-7.81 (m, 2H), 7.96 (d, 2H), 8.15 (d, 1H), 10.44 (s, 1H), 15.19 (br. s, 1H).

LC-MS (Method 1): Rₜ=1.23 min; MS (ESIpos): m/z=785.4 [M+H]⁺.

### Example 78A

### Methyl 2,2,3,3-tetrafluoro-3-[5-(4-nitrophenyl)-1H-1,2,4-triazol-3-yl]propanoate

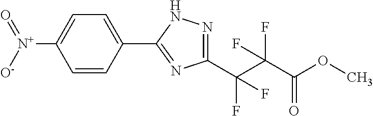 [SMILES: COC(=O)C(F)(F)C(F)(F)c1n[nH]c(-c2ccc([N+](=O)[O-])cc2)n1]

2,2,3,3-Tetrafluoro-3-[5-(4-nitrophenyl)-1H-1,2,4-triazol-3-yl]propanoic acid (30.3 g, 90.8 mmol) was dissolved in methanol (500 ml) and admixed with concentrated sulphuric acid (3 ml). The mixture was stirred at 65° C. for 22 h. Then concentrated sulphuric acid (5 ml) was added and the mixture was stirred once again at 65° C. for 22 h. Sodium hydrogencarbonate was added at RT to pH=7, the mixture was filtered and the solvent was removed under reduced pressure. The residue was stirred in petroleum ether and diethyl ether and then filtered. This gave 31.6 g (77% of theory) of the title compound.

LC-MS (Method 1): Rₜ=0.96 min; MS (ESIpos): m/z=349.1 [M+H]⁺.

### Example 79A

### Methyl 3-[5-(4-aminophenyl)-1H-1,2,4-triazol-3-yl]-2,2,3,3-tetrafluoropropanoate

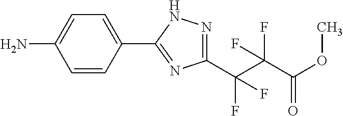 [SMILES: COC(=O)C(F)(F)C(F)(F)c1n[nH]c(-c2ccc(N)cc2)n1]

Methyl 2,2,3,3-tetrafluoro-3-[5-(4-nitrophenyl)-1H-1,2,4-triazol-3-yl]propanoate (24.0 g, 68.9 mmol) was initially charged in THF (370 ml) and admixed with palladium/charcoal (10%, 50% water-moist) under an argon atmosphere. Hydrogenation was effected with hydrogen (1 bar) at RT for 18 h. The mixture was filtered through kieselguhr and washed with dichloromethane/methanol 9:1. The filtrate was concentrated and the residue was dried under reduced pressure. This gave 21.7 g (99% of theory) of the title compound.

LC-MS (Method 1): Rₜ=0.78 min; MS (ESIpos): m/z=319.1 [M+H]⁺.

### Example 80A

### 3-{5-[4-({(2S)-2-{[(trans-4-{[(tert-Butoxycarbonyl)amino]methyl}cyclohexyl)carbonyl]amino}-3-[4′-methoxy-3′-(morpholin-4-ylsulphonyl)biphenyl-4-yl]propanoyl}amino)phenyl]-1H-1,2,4-triazol-3-yl}-2,2,3,3-tetrafluoropropanoic acid

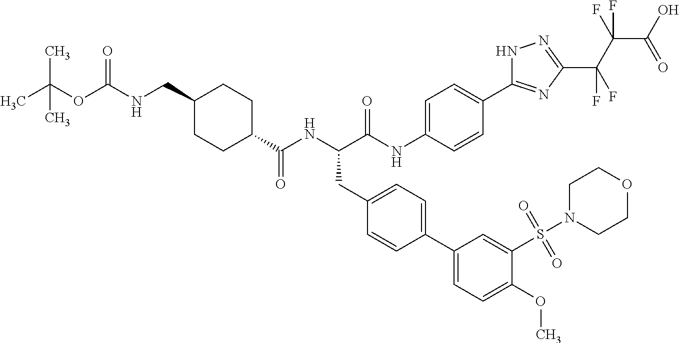 [SMILES: COc1ccc(-c2ccc(C[C@H](NC(=O)[C@H]3CC[C@H](CNC(=O)OC(C)(C)C)CC3)C(=O)Nc3ccc(-c4nc(C(F)(F)C(F)(F)C(=O)O)n[nH]4)cc3)cc2)cc1S(=O)(=O)N1CCOCC1]

Methyl 3-{5-[4-({4-bromo-N-[(trans-4-{[(tert-butoxycarbonyl)amino]methyl}cyclohexyl)-carbonyl]-L-phenylalanyl}amino)phenyl]-1H-1,2,4-triazol-3-yl}-2,2,3,3-tetrafluoropropanoate (125 mg, 0.16 mmol) and [4-methoxy-3-(morpholin-4-ylsulphonyl)phenyl]boric acid (96 mg, 0.32 mmol) were dissolved in 1.3 ml dimethylformamide and admixed with aqueous sodium carbonate solution (2M, 0.16 ml, 0.32 mmol) and degassed. After addition of 12 mg (0.02 mmol) of 1,1′-bis(diphenylphosphino)ferrocenepalladium(II) chloride, the reaction mixture was stirred at 120° C. for 30 min. The reaction solution was filtered through a Millipore syringe filter and purified via preparative HPLC (mobile phase: acetonitrile/water with 0.1% trifluoroacetic acid (gradient)). This gave 97 mg (62% of theory) of the title compound.

LC-MS (Method 1): Rₜ=0.96 min; MS (ESIpos): m/z=946.4 [M+H]⁺.

### Example 81A

### 4-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide

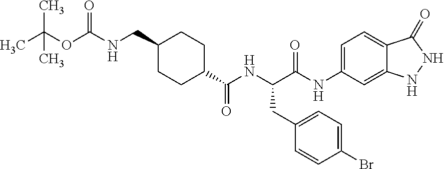

N,N-Diisopropylethylamine is added to a solution of 4-bromo-N-[(trans-4-{[(tert-butoxycarbonyl)amino]methyl}cyclohexyl)carbonyl]-L-phenylalanine and 6-amino-1,2-dihydro-3H-indazol-3-one in ethyl acetate. A 2,4,6-tripropyl-1,3,5,2,4,6-trioxatriphosphinane 2,4,6-trioxide solution (50% in dimethylformamide) is added to the reaction mixture, and the mixture is then stirred overnight at RT. The reaction mixture is worked up by methods known to those skilled in the art and the residue is separated by means of preparative HPLC. This gives the title compound.

### Example 82A

### tert-Butyl {[trans-4-({(2S)-3-[2′-methyl-5′-(pyrrolidin-1-ylsulphonyl)biphenyl-4-yl]-1-oxo-1-[(3-oxo-2,3-dihydro-1H-indazol-6-yl)amino]propan-2-yl}carbamoyl)cyclohexyl]methyl}carbamate

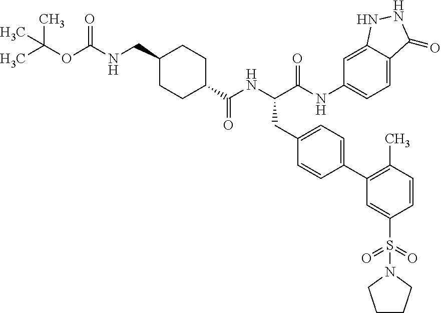 [SMILES: Cc1ccc(S(=O)(=O)N2CCCC2)cc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [2-methyl-5-(pyrrolidin-1-ylsulphonyl)phenyl]boronic acid in DMSO. The mixture is treated in the microwave at 110° C. for 1 to 3 hours. The mixture is filtered and purified by preparative HPLC. This gives the title compound.

### Example 83A

### tert-Butyl {[trans-4-({(2S)-3-[5′-(dimethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate

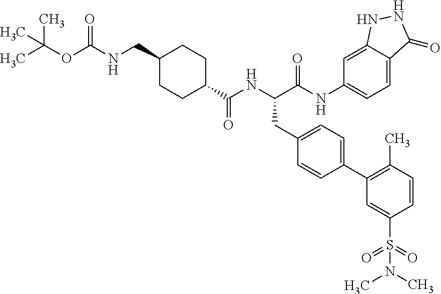 [SMILES: Cc1ccc(S(=O)(=O)N(C)C)cc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [5-(dimethyl sulphamoyl)-2-methylphenyl]boronic acid in DMSO. The mixture is treated in the microwave at 110° C. for 1 to 3 hours. The mixture is filtered and purified by preparative HPLC. This gives the title compound.

### Example 84A

### tert-Butyl {[trans-4-({(2S)-3-[2′-methyl-5′-(morpholin-4-ylsulphonyl)biphenyl-4-yl]-1-oxo-1-[(3-oxo-2,3-dihydro-1H-indazol-6-yl)amino]propan-2-yl}carbamoyl)cyclohexyl]methyl}carbamate

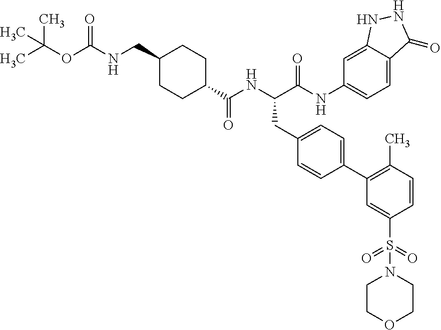 [SMILES: Cc1ccc(S(=O)(=O)N2CCOCC2)cc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [2-methyl-5-(morpholin-4-ylsulphonyl)phenyl]boronic acid in DMSO. The mixture is treated in the microwave at 110° C. for 1 to 3 hours. The mixture is filtered and purified by preparative HPLC. This gives the title compound.

### Working Examples

### Example 1

### trans-4-(Aminomethyl)-N-[(2S)-3-[5′-(dimethylsulphamoyl)-2′-methylbiphenyl-4-yl]-1-oxo-1-{[4-(1H-tetrazol-5-yl)phenyl]amino}propan-2-yl]cyclohexanecarboxamide hydrochloride

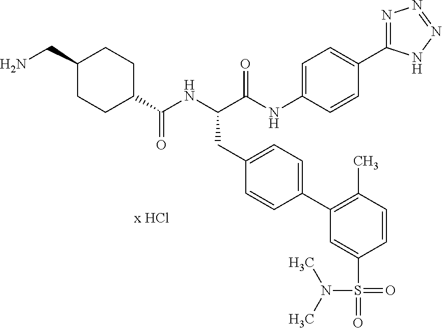

0.19 ml (0.77 mmol) of 4M hydrogen chloride in 1,4-dioxane was added to a suspension of 57 mg (0.077 mmol) of tert-butyl [(trans-4-{[(2S)-3-[5′-(dimethylsulphamoyl)-2′-methylbiphenyl-4-yl]-1-oxo-1-{[4-(1H-tetrazol-5-yl)phenyl]amino}propan-2-yl]carbamoyl}cyclohexyl)methyl]carbamate in 2.3 ml of 1,4-dioxane, and the mixture was stirred at RT overnight. After further addition of 0.06 ml (0.23 mmol) of 4M hydrogen chloride in 1,4-dioxane and stirring at RT for 48 h, 5 ml of acetonitrile were added to the reaction mixture and the precipitate formed was filtered off with suction, taken up in water and lyophilized. This gave 35 mg (63% of theory) of the title compound.

¹H-NMR (300 MHz, DMSO-d₆): δ ppm 0.74-1.00 (m, 2H), 1.02-1.31 (m, 2H), 1.34-1.47 (m, 1H), 1.48-1.58 (m, 1H), 1.63-1.80 (m, 3H), 2.04-2.17 (m, 1H), 2.24 (s, 3H), 2.55-2.64 (m, 8H), 2.92 (dd, 1H), 3.11 (dd, 1H), 4.68-4.79 (m, 1H), 7.28 (d, 2H), 7.38 (m, 3H), 7.54 (d, 1H), 7.57-7.63 (m, 1H), 7.70 (br. s, 3H), 7.79 (d, 2H), 7.97 (d, 2H), 8.24 (d, 1H), 10.49 (br. s, 1H).

LC-MS (Method 4): Rₜ=0.86 min; MS (ESIpos): m/z=645 [M+H-HCl]⁺.

### Example 2

### trans-4-(Aminomethyl)-N-[(2S)-3-[2′-methyl-5′-(morpholin-4-ylsulphonyl)biphenyl-4-yl]-1-oxo-1-{[4-(1H-tetrazol-5-yl)phenyl]amino}propan-2-yl]cyclohexanecarboxamide hydrochloride

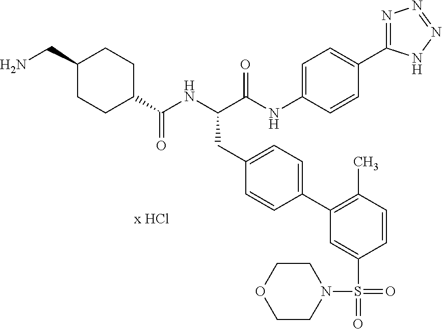

0.3 ml (1.3 mmol) of 4M hydrogen chloride in 1,4-dioxane was added to a suspension of 100 mg (0.13 mmol) of tert-butyl [(trans-4-{[(2S)-3-[2′-methyl-5′-(morpholin-4-ylsulphonyl)biphenyl-4-yl]-1-oxo-1-{[4-(1H-tetrazol-5-yl)phenyl]amino}propan-2-yl]carbamoyl}cyclohexyl)methyl]carbamate in 4.0 ml of 1,4-dioxane, and the mixture was stirred at RT overnight. After further addition of 0.1 ml (0.39 mmol) of 4M hydrogen chloride in 1,4-dioxane and stirring at RT for 48 h, 5 ml of acetonitrile were added to the reaction mixture and the precipitate formed was filtered off with suction, taken up in water and lyophilized. This gave 70 mg (71% of theory) of the title compound.

¹H-NMR (300 MHz, DMSO-d₆): δ ppm 0.78-0.97 (m, 2H), 1.04-1.31 (m, 2H), 1.34-1.47 (m, 1H), 1.48-1.58 (m, 1H), 1.63-1.77 (m, 3H), 2.05-2.18 (m, 1H), 2.24 (s, 3H), 2.56-2.64 (m, 2H), 2.79-2.86 (m, 4H), 2.92 (dd, 1H), 3.11 (dd, 1H), 3.55-3.63 (m, 4H), 4.66-4.78 (m, 1H), 7.25-7.32 (m, 2H), 7.35-7.41 (m, 3H), 7.53-7.61 (m, 2H), 7.63-7.73 (m, 3H), 7.79 (d, 2H), 7.97 (d, 2H), 8.24 (d, 1H), 10.47 (s, 1H).

LC-MS (Method 4): Rₜ=0.85 min; MS (ESIpos): m/z=687 [M+H-HCl]⁺.

### Example 3

### trans-4-(Aminomethyl)-N-{(2S)-1-{[4-(5-chloro-4H-1,2,4-triazol-3-yl)phenyl]amino}-3-[5′-(dimethyl sulphamoyl)-2′-methylbiphenyl-4-yl]-1-oxopropan-2-yl}cyclohexanecarboxamide hydrochloride

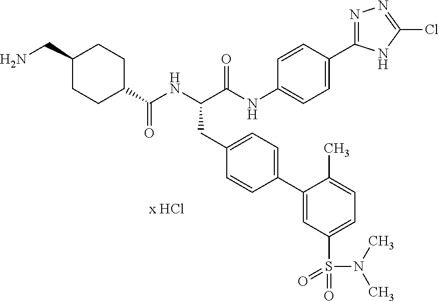

0.07 ml (0.3 mmol) of 4M hydrogen chloride in 1,4-dioxane was added to a suspension of 30 mg (0.04 mmol) of tert-butyl {[trans-4-({(2S)-1-{[4-(5-chloro-4H-1,2,4-triazol-3-yl)phenyl]amino}-3-[5′-(dimethyl sulphamoyl)-2′-methylbiphenyl-4-yl]-1-oxopropan-2-yl}carbamoyl)cyclohexyl]methyl}carbamate in 1.1 ml of 1,4-dioxane, and the mixture was stirred at RT overnight. After further addition of 0.1 ml (0.39 mmol) of 4M hydrogen chloride in 1,4-dioxane and 24 h of stirring at RT and 2 h at 40° C., 5 ml of acetonitrile were added to the reaction mixture and the precipitate formed was filtered off with suction and purified chromatographically by HPLC (Method 10). This gave 10 mg (33% of theory) of the title compound.

¹H-NMR (300 MHz, DMSO-d₆): δ ppm 0.77-0.94 (m, 2H), 1.03-1.28 (m, 2H), 1.28-1.43 (m, 1H), 1.47-1.58 (m, 1H), 1.62-1.78 (m, 3H), 2.04-2.16 (m, 1H), 2.24 (s, 3H), 2.52-2.56 (m, 2H), 2.58 (s, 6H), 2.91 (dd, 2H), 3.10 (dd, 1H), 4.65-4.78 (m, 1H), 7.27 (d, 2H), 7.34-7.41 (m, 3H), 7.53 (d, 1H), 7.57-7.67 (m, 3H), 7.82 (d, 2H), 8.16 (d, 1H), 8.32 (s, 1H), 10.23 (s, 1H).

LC-MS (Method 4): Rₜ=0.95 min; MS (ESIpos): m/z=680 [M+H-HCl]⁺.

### Example 4

### trans-4-(Aminomethyl)-N-[(2S)-1-oxo-3-(3′-sulphamoylbiphenyl-4-yl)-1-{[4-(1H-tetrazol-5-yl)phenyl]amino}propan-2-yl]cyclohexanecarboxamide hydrochloride

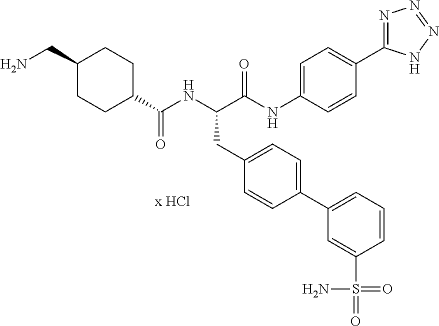

0.23 ml (0.9 mmol) of 4M hydrogen chloride in 1,4-dioxane was added to a suspension of 91 mg (0.13 mmol) of tert-butyl [(trans-4-{[(2S)-1-oxo-3-(3′-sulphamoylbiphenyl-4-yl)-1-{[4-(1H-tetrazol-5-yl)phenyl]amino}propan-2-yl]carbamoyl}cyclohexyl)methyl]carbamate in 4.0 ml of 1,4-dioxane, and the mixture was stirred at RT overnight. 5 ml of acetonitrile were then added to the reaction mixture and the resulting precipitate was filtered off with suction and dried under high vacuum. This gave 64 mg (76% of theory) of the title compound.

¹H-NMR (300 MHz, DMSO-d₆): δ ppm 0.76-0.98 (m, 2H), 1.05-1.31 (m, 2H), 1.35-1.50 (m, 1H), 1.51-1.61 (m, 1H), 1.64-1.77 (m, 3H), 2.06-2.17 (m, 1H), 2.55-2.64 (m, 2H), 2.91 (dd, 1H), 3.10 (dd, 1H), 7.37 (s, 1H), 7.43 (d, 2H), 7.56-7.65 (m, 3H), 7.78 (m, 7H), 7.99 (d, 2H), 8.06 (s, 1H), 8.25 (d, 1H), 10.56 (s, 1H).

LC-MS (Method 4): Rₜ=0.71 min; MS (ESIpos): m/z=603.5 [M+H-HCl]⁺.

### Example 5

trans-4-(Aminomethyl)-N-[(2S)-3-{3′-[(4-methylpiperazin-1-yl)carbonyl]biphenyl-4-yl}-1-oxo-1-{[4-(1H-tetrazol-5-yl)phenyl]amino}propan-2-yl]cyclohexanecarboxamide hydrochloride

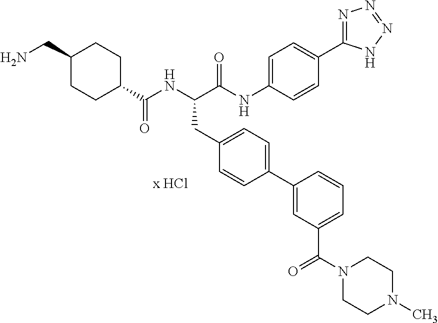 [SMILES: CN1CCN(C(=O)c2cccc(-c3ccc(C[C@H](NC(=O)[C@H]4CC[C@H](CN)CC4)C(=O)Nc4ccc(-c5nnn[nH]5)cc4)cc3)c2)CC1]

173 μl (0.69 mmol) of 4M hydrogen chloride in dioxane were added to a solution of 51.8 mg (69 μmol) of tert-butyl [(trans-4-{[(2S)-3-{3′-[(4-methylpiperazin-1-yl)carbonyl]biphenyl-4-yl}-1-oxo-1-{[4-(1H-tetrazol-5-yl)phenyl]amino}propan-2-yl]carbamoyl}cyclohexyl)methyl]carbamate formate in 3 ml of dioxane. The mixture was stirred at 35° C. overnight. Acetonitrile was added to the contents of the flask. The precipitated product was filtered off with suction, washed with acetonitrile and dried under high vacuum. 41 mg (85% of theory) of the title compound were obtained.

LC-MS (Method 5): Rₜ=0.58 min; MS (ESIpos): m/z=650 [M+H-HCl]⁺.

¹H-NMR (300 MHz, DMSO-d₆) δ ppm 0.77-0.98 (m, 2H), 1.04-1.30 (m, 2H), 1.32-1.62 (m, 2H), 1.64-1.79 (m, 3H), 2.05-2.19 (m, 1H), 2.58 (m, 2H), 2.73 (s, 3H), 2.84-2.97 (m, 1H), 3.04-3.14 (m, 3H), 4.62-4.76 (m, 1H), 7.40 (m, 3H), 7.47-7.55 (m, 1H), 7.60 (d, 2H), 7.66 (s, 1H), 7.70-7.88 (m, 5H), 7.99 (d, 2H), 8.26 (s, 1H), 10.57 (s, 1H).

### Example 6

### trans-4-(Aminomethyl)-N-[(2S)-3-{3′-[2-(morpholin-4-yl)ethoxy]biphenyl-4-yl}-1-oxo-1-{[4-(2H-tetrazol-5-yl)phenyl]amino}propan-2-yl]cyclohexanecarboxamide hydrochloride

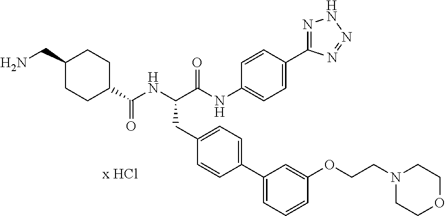

0.47 ml (1.9 mmol) of 4M hydrogen chloride in 1,4-dioxane was added to a solution of 108 mg (0.13 mmol) of tert-butyl [(trans-4-{[(2S)-3-{3′-[2-(morpholin-4-yl)ethoxy]biphenyl-4-yl}-1-oxo-1-{[4-(2H-tetrazol-5-yl)phenyl]amino}propan-2-yl]carbamoyl}cyclohexyl)methyl]carbamate trifluoroacetate in 3 ml of 1,4-dioxane, and the mixture was stirred at RT overnight. The precipitate was filtered off, washed with a little acetonitrile and dried under high vacuum. This gave 76 mg (79% of theory) of the title compound.

¹H-NMR (400 MHz, DMSO-d₆): δ=0.85-0.99 (m, 2H), 1.13-1.34 (m, 2H), 1.42-1.65 (m, 2H), 1.70-1.82 (m, 3H), 2.10-2.21 (m, 1H), 2.58-2.70 (m, 2H), 2.93 (dd, 1H), 3.07-3.28 (m, 3H), 3.42-4.03 (m, 8H), 4.47-4.54 (m, 2H), 4.68-4.76 (m, 1H), 6.98 (d, 1H), 7.22-7.29 (m, 2H), 7.36-7.44 (m, 3H), 7.50 (d, 2H), 7.76-7.90 (m, 5H), 8.02 (d, 2H), 8.30 (d, 1H), 10.61 (s, 1H), 11.1 (bs, 1H).

LC-MS (Method 1): Rₜ=0.53 min; MS (ESIneg): m/z=651 [M−H-HCl]⁻.

### Example 7

### trans-4-(Aminomethyl)-N-{(2S)-3-[2′-methyl-5′-(pyrrolidin-1-ylsulphonyl)biphenyl-4-yl]-1-oxo-1-[(2-oxo-2,3-dihydro-1H-benzimidazol-5-yl)amino]propan-2-yl}cyclohexanecarboxamide hydrochloride

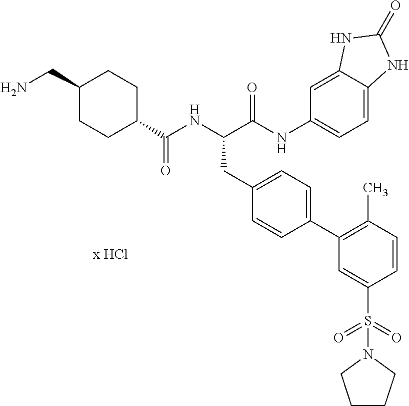 [SMILES: Cc1ccc(S(=O)(=O)N2CCCC2)cc1-c1ccc(C[C@H](NC(=O)[C@H]2CC[C@H](CN)CC2)C(=O)Nc2ccc3[nH]c(=O)[nH]c3c2)cc1]

118 μl (0.47 mmol) of 4M hydrogen chloride in dioxane were added to a solution of 71.5 mg (94 μmol) of tert-butyl {[trans-4-({(2S)-3-[2′-methyl-5′-(pyrrolidin-1-ylsulphonyl)biphenyl-4-yl]-1-oxo-1-[(2-oxo-2,3-dihydro-1H-benzimidazol-5-yl)amino]propan-2-yl}carbamoyl)cyclohexyl]methyl}carbamate in 2.5 ml of dioxane. The mixture was stirred at RT for 4 days. Acetonitrile was added to the contents of the flask. The precipitated product was filtered off with suction, washed with acetonitrile and dried under high vacuum. The residue was purified by preparative HPLC (Method 11). 14 mg (21% of theory) of the title compound were obtained.

LC-MS (Method 5): Rₜ=1.09 min; MS (ESIpos): m/z=659 [M+H-HCl]⁺.

¹H-NMR (400 MHz, DMSO-d₆) δ ppm 0.73-0.89 (m, 2H), 1.01-1.05 (m, 1H), 1.06-1.32 (m, 3H), 1.46-1.55 (m, 1H), 1.66 (br. s., 7H), 2.01-2.15 (m, 1H), 2.26 (s, 3H), 2.34-2.39 (m, 1H), 2.85-2.95 (m, 1H), 3.09-3.16 (m, 4H), 3.45-3.57 (m, 2H), 4.65-4.76 (m, 1H), 6.82 (d, 1H), 6.98 (dd, 1H), 7.28 (d, 2H), 7.37 (d, 2H), 7.41-7.43 (m, 1H), 7.44-7.48 (m, 1H), 7.54 (d, 1H), 7.67 (dd, 1H), 8.05 (d, 1H), 9.92 (s, 1H), 10.49 (br. s, 1H).

### Example 8

### Ethyl 4′-[(2S)-2-({[trans-4-(aminomethyl)cyclohexyl]carbonyl}amino)-3-oxo-3-{[4-(2H-tetrazol-5-yl)phenyl]amino}propyl]-2-fluorobiphenyl-3-carboxylate hydrochloride

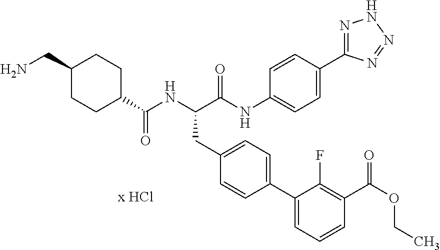

0.26 ml (1.05 mmol) of 4M hydrogen chloride in dioxane was added to a solution of 50 mg (0.07 mmol) of ethyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-fluorobiphenyl-3-carboxylate in 2 ml of 1,4-dioxane, and the mixture was stirred at RT overnight. The precipitate was filtered off, washed with a little acetonitrile and dried under high vacuum. This gave 39 mg (80% of theory) of the title compound.

¹H-NMR (400 MHz, DMSO-d₆) δ=0.84-1.01 (m, 2H), 1.09-1.37 (m, 5H), 1.40-1.66 (m, 2H), 1.68-1.83 (m, 3H), 2.10-2.20 (m, 1H), 2.58-2.68 (m, 2H), 2.90-3.01 (m, 1H), 3.08-3.21 (m, 1H), 4.29-4.39 (m, 2H), 4.68-4.80 (m, 1H), 7.35-7.51 (m, 5H), 7.67-7.90 (m, 7H), 8.02 (d, 2H), 8.28 (d, 1H), 10.58 (s, 1H).

LC-MS (Method 1): Rₜ=0.79 min; MS (ESIneg): m/z=612 [M−H-HCl]⁻.

### Example 9

### 4-[(2S)-2-({[trans-4-(Aminomethyl)cyclohexyl]carbonyl}amino)-3-oxo-3-{[4-(2H-tetrazol-5-yl)phenyl]amino}propyl]-6-methyl-N-(piperidin-4-yl)biphenyl-3-carboxamide hydrochloride

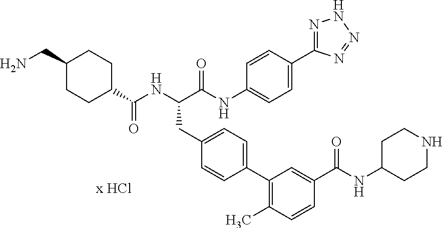

0.25 ml (1.01 mmol) of 4M hydrogen chloride in 1,4-dioxane was added to a solution of 66 mg (0.7 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-methylbiphenyl-3-yl}carbonyl)amino]piperidine-1-carboxy late in 2.5 ml of 1,4-dioxane, and the mixture was stirred at RT overnight. The precipitate was filtered off, washed with a little acetonitrile and dried under high vacuum. This gave 54 mg (99% of theory, 91% purity) of the title compound.

¹H-NMR (400 MHz, DMSO-d₆) δ=0.84-1.00 (m, 2H), 1.09-1.35 (m, 2H), 1.41-1.64 (m, 2H), 1.66-1.84 (m, 5H), 1.88-2.00 (m, 2H), 2.11-2.26 (m, 4H), 2.58-2.68 (m, 2H), 2.90-3.06 (m, 3H), 3.09-3.19 (m, 1H), 3.23-3.35 (m, 2H), 3.99-4.12 (m, 1H), 4.69-4.81 (m, 1H), 7.27 (d, 2H), 7.33-7.46 (m, 3H), 7.66-7.71 (m, 1H), 7.73-7.93 (m, 6H), 8.02 (d, 2H), 8.31 (d, 1H), 8.48 (d, 1H), 8.67-8.90 (m, 2H), 10.59 (s, 1H).

LC-MS (Method 1): Rₜ=0.56 min; MS (ESIneg): m/z=662 [M−H-HCl]⁻.

### Example 10

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[2-(diethylamino)ethyl]-6-methylbiphenyl-3-carboxamide hydrochloride

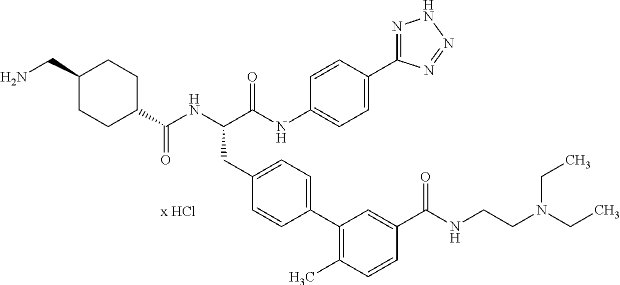

0.25 ml (1.01 mmol) of 4M hydrogen chloride in 1,4-dioxane was added to a solution of 60 mg (0.07 mmol) of tert-butyl [(trans-4-{[(2S)-3-(5′-{[2-(diethylamino)ethyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate in 2.5 ml of 1,4-dioxane, and the mixture was stirred at RT overnight. The precipitate was filtered off, washed with a little acetonitrile and dried under high vacuum. This gave 44 mg (87% of theory) of the title compound.

¹H-NMR (400 MHz, DMSO-d₆) δ=0.85-1.01 (m, 2H), 1.12-1.34 (m, 8H), 1.40-1.64 (m, 2H), 1.67-1.83 (m, 3H), 2.10-2.28 (m, 4H), 2.59-2.69 (m, 2H), 2.89-3.02 (m, 1H), 3.08-3.26 (m, 7H), 3.59-3.66 (m, 2H), 4.71-4.81 (m, 1H), 7.29 (d, 2H), 7.35-7.45 (m, 3H), 7.71 (s, 1H), 7.74-7.92 (m, 6H), 8.02 (d, 2H), 8.31 (dl H), 8.80-8.90 (m, 1H), 10.0 (br. s, 1H), 10.58 (s, 1H).

LC-MS (Method 1): Rₜ=0.57 min; MS (ESIneg): m/z=678 [M−H-HCl]⁻.

### Example 11

### 4′-[(2S)-2-({[trans-4-(Aminomethyl)cyclohexyl]carbonyl}amino)-3-oxo-3-{[4-(2H-tetrazol-5-yl)phenyl]amino}propyl]-6-methyl-N-[(3R)-pyrrolidin-3-yl]biphenyl-3-carboxamide hydrochloride

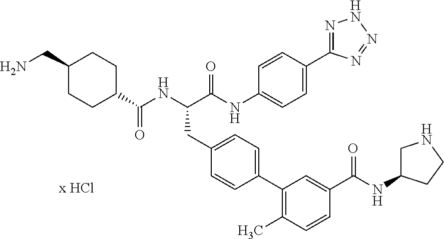

0.24 ml (0.95 mmol) of 4M hydrogen chloride in 1,4-dioxane was added to a solution of 61 mg (0.06 mmol) of tert-butyl (3R)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-methylbiphenyl-3-yl}carbonyl)amino]pyrrolidine-1-carboxylate in 2.5 ml of 1,4-dioxane, and the mixture was stirred at RT overnight. The precipitate was filtered off, washed with a little acetonitrile, dried under high vacuum and then separated by preparative HPLC (mobile phase: gradient of acetonitrile/water with 0.1% trifluoroacetic acid). The product-containing fractions were combined and admixed with 0.2 ml of 4M hydrogen chloride in 1,4-dioxane and the mixture was concentrated on a rotary evaporator. The residue was dried under high vacuum. This gave 25 mg (54% of theory) of the title compound.

¹H-NMR (400 MHz, DMSO-d₆) δ=0.82-1.01 (m, 2H), 1.11-1.35 (m, 2H), 1.40-1.64 (m, 2H), 1.67-1.83 (m, 3H), 1.91-2.03 (m, 1H), 2.09-2.27 (m, 5H), 2.58-2.69 (m, 2H), 2.90-3.02 (m, 1H), 3.08-3.43 (m, 5H), 4.47-4.59 (m, 1H), 4.69-4.80 (m, 1H), 7.29 (d, 2H), 7.35-7.46 (m, 3H), 7.71 (s, 1H), 7.74-7.92 (m, 5H), 8.02 (d, 2H), 8.31 (d, 1H), 8.69 (d, 1H), 9.1 (br. s, 1H), 9.3 (br. s, 1H), 10.59 (s, 1H).

LC-MS (Method 1): Rₜ=0.62 min; MS (ESIneg): m/z=648 [M−H-HCl]⁻.

### Example 12

### trans-4-(Aminomethyl)-N-[(2S)-3-[2′-methyl-5′-(piperazin-1-ylcarbonyl)biphenyl-4-yl]-1-oxo-1-{[4-(2H-tetrazol-5-yl)phenyl]amino}propan-2-yl]cyclohexanecarboxamide hydrochloride

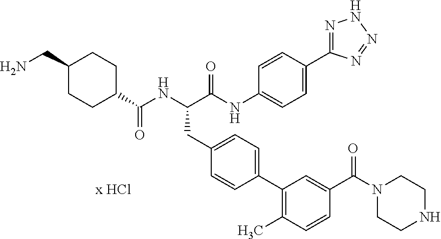

0.29 ml (1.17 mmol) of 4M hydrogen chloride in 1,4-dioxane was added to a solution of 75 mg (0.08 mmol) of tert-butyl 4-({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-6-methylbiphenyl-3-yl}carbonyl)piperazine-1-carboxylate in 2.5 ml of 1,4-dioxane, and the mixture was stirred at RT overnight. The precipitate was filtered off, washed with a little acetonitrile and dried under high vacuum. This gave 57 mg (93% of theory, 92% purity) of the title compound.

¹H-NMR (400 MHz, DMSO-d₆) δ=0.83-1.01 (m, 2H), 1.11-1.35 (m, 2H), 1.42-1.65 (m, 2H), 1.67-1.83 (m, 3H), 2.10-2.28 (m, 4H), 2.59-2.69 (m, 2H), 2.90-3.02 (m, 1H), 3.06-3.21 (m, 5H), 3.63-3.82 (m, 4H), 4.70-4.81 (m, 1H), 7.20-7.32 (m, 3H), 7.32-7.47 (m, 4H), 7.71-7.92 (m, 5H), 8.02 (d, 2H), 8.27 (d, 1H), 9.14-9.31 (m, 2H), 10.53 (s, 1H).

LC-MS (Method 1): Rₜ=0.56 min; MS (ESIneg): m/z=648 [M−H-HCl]⁻.

### Example 13

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(piperidin-4-yl)biphenyl-3-carboxamide hydrochloride

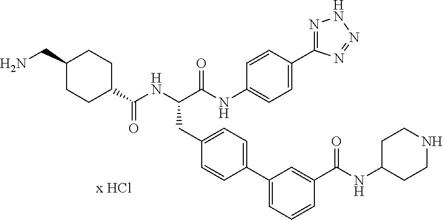

0.34 ml (1.34 mmol) of 4M hydrogen chloride in 1,4-dioxane was added to a solution of 86 mg (0.09 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-3-yl}carbonyl)amino]piperidine-1-carboxylate in 3 ml of 1,4-dioxane, and the mixture was stirred at RT overnight. The precipitate was filtered off, washed with a little acetonitrile, dried under high vacuum and then separated by preparative HPLC (mobile phase: gradient of acetonitrile/water with 0.1% trifluoroacetic acid). The product-containing fractions were combined and admixed with 0.2 ml of 4M hydrogen chloride in 1,4-dioxane and the mixture was concentrated on a rotary evaporator. The residue was dried under high vacuum. This gave 38 mg (58% of theory) of the title compound.

¹H-NMR (400 MHz, DMSO-d₆) δ=0.82-1.02 (m, 2H), 1.10-1.35 (m, 2H), 1.41-1.66 (m, 2H), 1.68-1.88 (m, 5H), 1.91-2.04 (m, 2H), 2.10-2.21 (m, 1H), 2.58-2.68 (m, 2H), 2.89-3.19 (m, 4H), 3.26-3.37 (m, 2H), 4.03-4.14 (m, 1H), 4.67-4.78 (m, 1H), 7.45 (d, 2H), 7.50-7.58 (m, 1H), 7.66 (d, 2H), 7.74-7.97 (m, 6H), 8.03 (d, 2H), 8.10 (s, 1H), 8.25-8.36 (m, 1H), 8.62 (d, 1H), 8.79-8.99 (m, 2H), 10.63 (s, 1H).

LC-MS (Method 1): Rₜ=0.60 min; MS (ESIneg): m/z=648 [M−H-HCl]⁻.

### Example 14

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[2-(diethylamino)ethyl]biphenyl-3-carboxamide hydrochloride

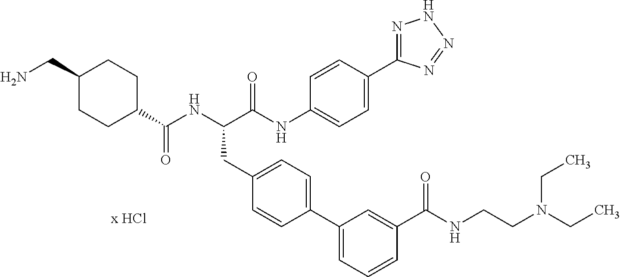

0.26 ml (1.06 mmol) of 4M hydrogen chloride in 1,4-dioxane was added to a solution of 62 mg (0.07 mmol) of tert-butyl [(trans-4-{[(2S)-3-(3′-{[2-(diethylamino)ethyl]carbamoyl}biphenyl-4-yl)-1-oxo-1-{[4-(2H-tetrazol-5-yl)phenyl]amino}propan-2-yl]carbamoyl}cyclohexyl)methyl]carbamate trifluoroacetate in 2.5 ml of 1,4-dioxane, and the mixture was stirred at RT overnight. The precipitate was filtered off, washed with a little acetonitrile and dried under high vacuum. This gave 42 mg (77% of theory) of the title compound.

¹H-NMR (400 MHz, DMSO-d₆) δ=0.83-1.02 (m, 2H), 1.11-1.35 (m, 8H), 1.41-1.67 (m, 2H), 1.68-1.83 (m, 3H), 2.10-2.22 (m, 1H), 2.58-2.69 (m, 2H), 2.88-3.01 (m, 1H), 3.07-3.30 (m, 7H), 3.63-3.72 (m, 2H), 4.67-4.79 (m, 1H), 7.44 (d 2H), 7.56 (t, 1H), 7.70 (d, 2H), 7.74-7.92 (m, 7H), 8.02 (d, 2H), 8.19 (s, 1H), 8.24-8.35 (m, 1H), 9.00-9.09 (m, 1H), 10.1 (br. s, 1H), 10.60 (s, 1H).

LC-MS (Method 1): Rₜ=0.56 min; MS (ESIneg): m/z=664[M−H-HCl]⁻.

### Example 15

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-fluoro-N-(piperidin-4-yl)biphenyl-3-carboxamide hydrochloride

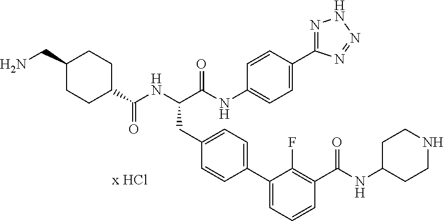

0.24 ml (0.96 mmol) of 4M hydrogen chloride in 1,4-dioxane was added to a solution of 63 mg (0.06 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-fluorobiphenyl-3-yl}carbonyl)amino]piperidine-1-carboxylate in 2.5 ml of 1,4-dioxane, and the mixture was stirred at RT overnight. The precipitate was filtered off, washed with a little acetonitrile and dried under high vacuum. This gave 56 mg (77% of theory, 92% purity) of the title compound.

¹H-NMR (400 MHz, DMSO-d₆) δ=0.83-1.01 (m, 2H), 1.11-1.34 (m, 2H), 1.42-1.65 (m, 2H), 1.66-1.85 (m, 5H), 1.92-2.04 (m, 2H), 2.09-2.21 (m, 1H), 2.58-2.69 (m, 2H), 2.90-3.08 (m, 3H), 3.09-3.19 (m, 1H), 3.20-3.32 (m, 2H), 3.98-4.12 (m, 1H), 4.66-4.79 (m, 1H), 7.33 (t, 1H), 7.46 (d, 5H), 7.72-7.94 (m, 5H), 8.02 (d, 2H), 8.31 (d, Hz, 1H), 8.58 (d, 1H), 8.64-8.89 (m, 2H), 10.60 (s, 1H), 16.8 (br. s, 1H).

LC-MS (Method 1): Rₜ=0.53 min; MS (ESIneg): m/z=666 [M−H-HCl]⁻.

### Example 16

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[2-(diethylamino)ethyl]-2-fluorobiphenyl-3-carboxamide hydrochloride

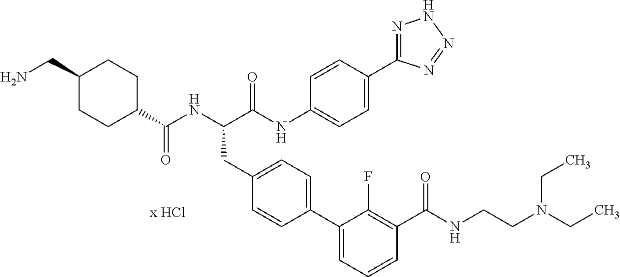

0.33 ml (1.32 mmol) of 4M hydrogen chloride in 1,4-dioxane was added to a solution of 79 mg (0.09 mmol) of tert-butyl [(trans-4-{[(2S)-3-(3′-{[2-(diethylamino)ethyl]carbamoyl}-2′-fluorobiphenyl-4-yl)-1-oxo-1-{[4-(2H-tetrazol-5-yl)phenyl]amino}propan-2-yl]carbamoyl}cyclohexyl)methyl]carbamate trifluoroacetate in 3 ml of 1,4-dioxane, and the mixture was stirred at RT overnight. The precipitate was filtered off, washed with a little acetonitrile and dried under high vacuum. This gave 64 mg (88% of theory, 92% purity) of the title compound.

¹H-NMR (400 MHz, DMSO-d₆) δ=0.84-1.01 (m, 2H), 1.12-1.35 (m, 8H), 1.42-1.55 (m, 1H), 1.56-1.66 (m, 1H), 1.69-1.83 (m, 3H), 2.11-2.21 (m, 1H), 2.59-2.68 (m, 2H), 2.92-3.02 (m, 1H), 3.09-3.29 (m, 7H), 3.61-3.70 (m, 2H), 4.68-4.79 (m, 1H), 7.36 (t, 1H), 7.41-7.53 (m, 4H), 7.56-7.69 (m, 2H), 7.75-7.95 (m, 5H), 8.03 (d, 2H), 8.31 (d, 1H), 8.68-8.80 (m, 1H), 10.2 (br. s, 1H), 10.61 (s, 1H).

LC-MS (Method 1): Rₜ=0.54 min; MS (ESIneg): m/z=682 [M−H-HCl]⁻.

### Example 17

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-fluoro-N-[(3R)-pyrrolidin-3-yl]biphenyl-3-carboxamide hydrochloride

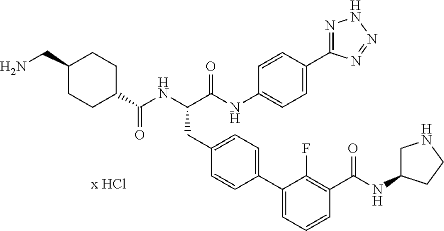

0.26 ml (1.04 mmol) of 4M hydrogen chloride in 1,4-dioxane was added to a solution of 67 mg (0.07 mmol) of tert-butyl (3R)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-fluorobiphenyl-3-yl}carbonyl)amino]pyrrolidine-1-carboxylate trifluoroacetate in 2 ml of 1,4-dioxane, and the mixture was stirred at RT overnight. The precipitate was filtered off, washed with a little acetonitrile and dried under high vacuum. This gave 48 mg (91% of theory) of the title compound.

¹H-NMR (400 MHz, DMSO-d₆) δ=0.84-1.00 (m, 2H), 1.12-1.34 (m, 2H), 1.41-1.54 (m, 1H), 1.55-1.66 (m, 1H), 1.68-1.83 (m, 3H), 1.88-2.00 (m, 1H), 2.10-2.26 (m, 2H), 2.59-2.68 (m, 2H), 2.90-3.01 (m, 1H), 3.07-3.18 (m, 2H), 3.19-3.49 (m, 3H), 4.47-4.57 (m, 1H), 4.68-4.79 (m, 1H), 7.35 (t, 1H), 7.40-7.52 (m, 4H), 7.54-7.64 (m, 2H), 7.72-7.90 (m, 5H), 8.02 (d, 2H), 8.30 (d, 1H), 8.76 (d, 1H), 9.02-9.29 (m, 2H), 10.59 (s, 1H), 16.8 (br. s, 1H).

LC-MS (Method 1): Rₜ=0.52 min; MS (ESIneg): m/z=652 [M−H-HCl]⁻.

### Example 18

### trans-4-(Aminomethyl)-N-[(2S)-3-[2′-fluoro-3′-(piperazin-1-ylcarbonyl)biphenyl-4-yl]-1-oxo-1-{[4-(2H-tetrazol-5-yl)phenyl]amino}propan-2-yl]cyclohexanecarboxamide hydrochloride

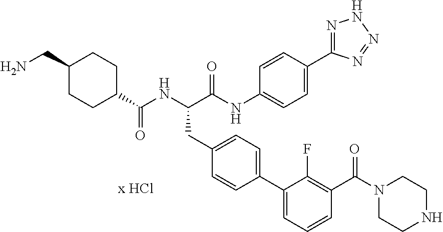

0.23 ml (0.91 mmol) of 4M hydrogen chloride in 1,4-dioxane was added to a solution of 59 mg (0.06 mmol) of tert-butyl 4-({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-fluorobiphenyl-3-yl}carbonyl)piperazine-1-carboxylate trifluoroacetate in 2 ml of 1,4-dioxane, and the mixture was stirred at RT overnight. The precipitate was filtered off, washed with a little acetonitrile and dried under high vacuum. This gave 42 mg (85% of theory, 90% purity) of the title compound.

¹H-NMR (400 MHz, DMSO-d₆) δ=0.83-1.01 (m, 2H), 1.10-1.34 (m, 2H), 1.42-1.66 (m, 2H), 1.67-1.84 (m, 3H), 2.10-2.21 (m, 1H), 2.59-2.69 (m, 2H), 2.90-3.00 (m, 1H), 3.01-3.26 (m, 5H), 3.49-3.56 (m, 2H), 3.76-4.01 (m, 2H), 4.68-4.80 (m, 1H), 7.33-7.54 (m, 6H), 7.56-7.64 (m, 1H), 7.74-7.93 (m, 5H), 8.03 (d, 2H), 8.30 (d, 1H), 9.3 (br. s, 2H), 10.60 (s, 1H), 16.8 (br. s, 1H).

LC-MS (Method 1): Rₜ=0.51 min; MS (ESIneg): m/z=652 [M−H-HCl]⁻.

### Example 19

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[3-(diethylamino)propyl]-6-methylbiphenyl-3-carboxamide hydrochloride

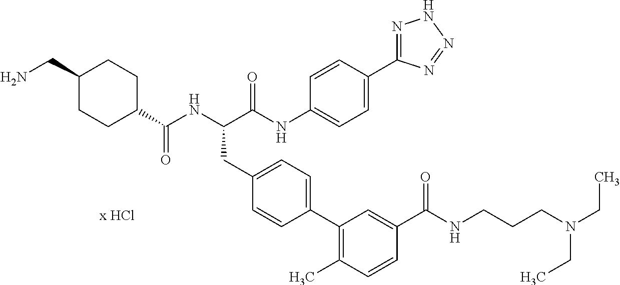

A solution of 73 mg (0.08 mmol) of tert-butyl [(trans-4-{[(2S)-3-(5′-{[3-(diethylamino)-propyl]carbamoyl}-2′-methylbiphenyl-4-yl)-1-oxo-1-{[4-(2H-tetrazol-5-yl)phenyl]amino}propan-2-yl]carbamoyl}cyclohexyl]methyl}carbamate trifluoroacetate in 2.5 ml of 1,4-dioxane was admixed with 0.30 ml (1.21 mmol) of 4M hydrogen chloride in 1,4-dioxane and stirred at RT overnight. The precipitate was filtered off, washed with a little acetonitrile and dried under high vacuum. This gave 51 mg (83% of theory) of the title compound.

¹H-NMR (400 MHz, DMSO-d₆) δ=0.85-1.00 (m, 2H), 1.11-1.34 (m, 8H), 1.41-1.63 (m, 2H), 1.69-1.82 (m, 3H), 1.83-1.94 (m, 2H), 2.10-2.27 (m, 4H), 2.60-2.67 (m, 2H), 2.90-3.01 (m, 1H), 3.02-3.19 (m, 7H), 3.26-3.38 (m, 2H), 4.71-4.81 (m, 1H), 7.29 (d, 2H), 7.35-7.44 (m, 3H), 7.66-7.70 (m, 1H), 7.72-7.90 (m, 6H), 7.99-8.06 (m, 2H), 8.31 (d, 1H), 8.62-8.70 (m, 1H), 9.9 (br. s, 1H), 10.58 (s, 1H).

LC-MS (Method 1): Rₜ=0.65 min; MS (ESIneg): m/z=692 [M−H-HCl]⁻.

### Example 20

### trans-4-(Aminomethyl)-N-[(2S)-3-[3′-(morpholin-4-ylcarbonyl)biphenyl-4-yl]-1-oxo-1-{[4-(2H-tetrazol-5-yl)phenyl]amino}propan-2-yl]cyclohexanecarboxamide hydrochloride

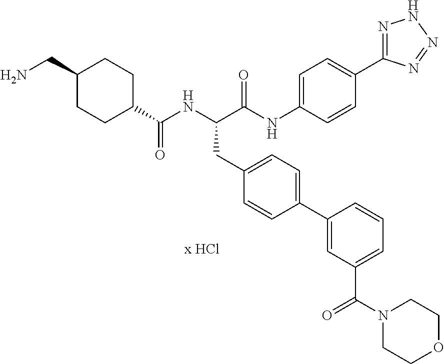

1.00 ml (4.00 mmol) of 4M hydrogen chloride in 1,4-dioxane was added to a solution of 91 mg (0.12 mmol) of tert-butyl [(trans-4-{[(2S)-3-[3′-(morpholin-4-ylcarbonyl)biphenyl-4-yl]-1-oxo-1-{[4-(2H-tetrazol-5-yl)phenyl]amino}propan-2-yl]carbamoyl}cyclohexyl)methyl]carbamate in 5 ml of tetrahydrofuran, and the mixture was stirred at RT for 16 h. The solvent was removed on a rotary evaporator and the residue was dried under high vacuum. The residue was triturated with acetonitrile, filtered, washed with acetonitrile and dried under high vacuum. 75 mg (84% of theory, 93% purity) of the title compound were obtained.

¹H-NMR (400 MHz, DMSO-d₆) δ=0.80-1.00 (m, 2H), 1.12-1.34 (m, 2H), 1.39-1.83 (m, 6H), 2.08-2.23 (m, 1H), 2.63 (m, 2H), 2.87-3.02 (m, 1H), 3.07-3.20 (m, 1H), 3.38 (d, 2H), 3.55-3.58 (m, 3H), 4.66-4.79 (m, 1H), 7.29-7.97 (m, 14H), 8.04 (d, 2H), 8.30 (d, 1H), 10.63 (br. s, 1H).

LC-MS (Method 1): Rₜ=0.64 min; MS (ESIneg): m/z=635 [M−H-HCl]⁻.

### Example 21

### trans-4-(Aminomethyl)-N-[(2S)-3-(5′-cyano-2′-methylbiphenyl-4-yl)-1-oxo-1-{[4-(2H-tetrazol-5-yl)phenyl]amino}propan-2-yl]cyclohexanecarboxamide hydrochloride

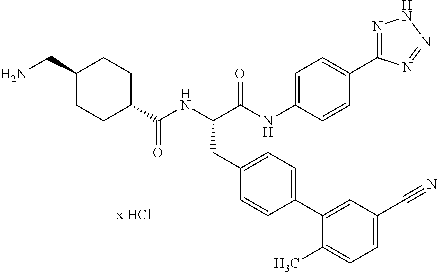 [SMILES: Cc1ccc(C#N)cc1-c1ccc(C[C@H](NC(=O)[C@H]2CC[C@H](CN)CC2)C(=O)Nc2ccc(-c3nn[nH]n3)cc2)cc1]

1.25 ml (5 mmol) of 4M hydrogen chloride in 1,4-dioxane was added to a solution of 70 mg (0.1 mmol) of tert-butyl [(trans-4-{[(2S)-3-(5′-cyano-T-methylbiphenyl-4-yl)-1-oxo-1-{[4-(2H-tetrazol-5-yl)phenyl]amino}propan-2-yl]carbamoyl}cyclohexyl)methyl]carbamate in 4 ml of tetrahydrofuran, and the mixture was stirred at RT for 4 h. A further 0.2 ml (1 mmol) of 4M hydrogen chloride in 1,4-dioxane was added, and the mixture was stirred at RT for 16 h. The solid formed was filtered, washed with tetrahydrofuran and acetonitrile and dried under high vacuum. 55.5 mg (85% of theory) of the title compound were obtained.

¹H-NMR (400 MHz, DMSO-d₆) δ=0.82-1.03 (m, 2H), 1.10-1.33 (m, 2H), 1.41-1.63 (m, 2H), 1.67-1.85 (m, 3H), 2.12-2.19 (m, 1H), 2.25 (s, 3H), 2.63 (t, 1H), 2.87-3.02 (m, 2H), 3.07-3.23 (m, 1H), 4.67-4.80 (m, 1H), 7.30 (d, 2H), 7.42 (d, 2H), 7.51 (d, 1H), 7.60 (d, 1H), 7.68-7.91 (m, 6H), 8.02 (d, 1H), 8.31 (d, 1H), 10.58 (br. s., 1H).

LC-MS (Method 1): Rₜ=0.74 min; MS (ESIneg): m/z=561 [M−H-HCl]⁻.

### Example 22

### N-{4[(2S)-2-({[trans-4-(Aminomethyl)cyclohexyl]carbonyl}amino)-3-oxo-3-{[4-(2H-tetrazol-5-yl)phenyl]amino}propyl]-4-methylbiphenyl-3-yl}piperidine-4-carboxamide hydrochloride

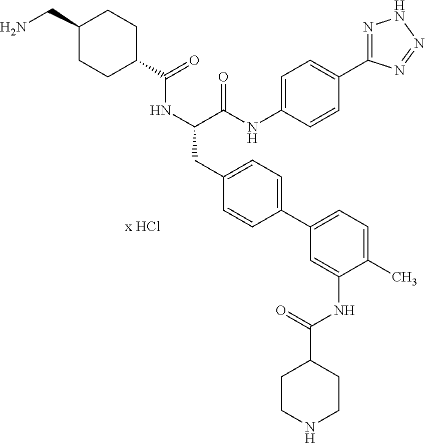 [SMILES: Cc1ccc(-c2ccc(C[C@H](NC(=O)[C@H]3CC[C@H](CN)CC3)C(=O)Nc3ccc(-c4nn[nH]n4)cc3)cc2)cc1NC(=O)C1CCNCC1]

0.07 ml (0.26 mmol) of 4M hydrogen chloride in dioxane was added to a solution of 17.2 mg (0.018 mmol) of tert-butyl 4-({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-4-methylbiphenyl-3-yl}carbamoyl)piperidine-1-carboxylate trifluoroacetate in 1 ml of dioxane. The mixture was stirred at RT for 7 d. The precipitated product was filtered off with suction, washed with dioxane and dried under high vacuum. 14 mg (100% of theory) of the title compound were obtained.

LC-MS (Method 1): Rₜ=0.60 min; MS (ESIpos): m/z=664 [M+H-HCl]⁺.

¹H-NMR (400 MHz, DMSO-d₆) δ=0.85-0.98 (m, 2H), 1.11-1.32 (m, 2H), 1.44-1.53 (m, 1H), 1.60 (d, 1H), 1.70-1.92 (m, 5H), 1.96-2.04 (m, 2H), 2.15 (br. s., 1H), 2.21 (s, 3H), 2.63 (t, 2H), 2.75 (br. s., 1H), 2.87-2.99 (m, 3H), 3.06-3.15 (m, 1H), 4.72 (m, 1H), 7.28 (d, 1H), 7.39 (d, 3H), 7.53 (d, 2H), 7.63 (s, 1H), 7.84 (d, 4H), 8.02 (d, 2H), 8.26 (d, 1H), 8.56-8.72 (m, 1H), 8.80-9.04 (m, 1H), 9.54 (s, 1H), 10.57 (br. s., 1H).

### Example 23

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[2-(diethylamino)ethyl]-6-ethylbiphenyl-3-carboxamide hydrochloride

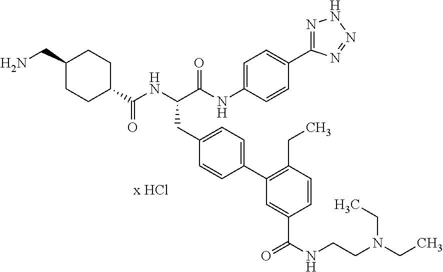 [SMILES: CCc1ccc(C(=O)NCCN(CC)CC)cc1-c1ccc(C[C@H](NC(=O)[C@H]2CC[C@H](CN)CC2)C(=O)Nc2ccc(-c3nn[nH]n3)cc2)cc1]

To a solution of 60 mg (0.07 mmol) of tert-butyl [(trans-4-{[(2S)-3-(5′-{[2-(diethylamino)ethyl]carbamoyl}-2′-ethylbiphenyl-4-yl)-1-oxo-1-{[4-(2H-tetrazol-5-yl)phenyl]amino}propan-2-yl]carbamoyl}cyclohexyl)methyl]carbamate trifluoroacetate in 2.5 ml of dioxane was added 0.25 ml (0.99 mmol) of 4M hydrogen chloride in dioxane. The mixture was stirred at RT for 16 h. The precipitated product was filtered off with suction, washed with acetonitrile and dried under high vacuum. 48 mg (91% of theory) of the title compound were obtained.

LC-MS (Method 1): Rₜ=0.61 min; MS (ESIneg): m/z=694 [M+H-HCl]⁺.

¹H-NMR (400 MHz, DMSO-d₆) δ ppm 0.86-1.05 (m, 6H), 1.22 (m, 9H), 1.40-1.54 (m, 1H), 1.55-1.63 (m, 1H), 1.67-1.82 (m, 3H), 2.10-2.21 (m, 1H), 2.63 (t, 2H), 2.91-3.02 (m, 1H), 3.11-3.26 (m, 7H), 3.59-3.66 (m, 2H), 4.70-4.82 (m, 1H), 7.25 (d, 2H), 7.42 (dd, 3H), 7.67 (d, 1H), 7.85 (m, 6H), 8.03 (d, 2H), 8.32 (d, 1H), 8.86 (t, 1H), 10.14 (s, 1H), 10.60 (s, 1H).

### Example 24

### trans-4-(Aminomethyl)-N-[(2S)-3-[4′-methoxy-3′-(morpholin-4-ylsulphonyl)biphenyl-4-yl]-1-oxo-1-{[4-(1H-tetrazol-5-yl)phenyl]amino}propan-2-yl]cyclohexanecarboxamide hydrochloride

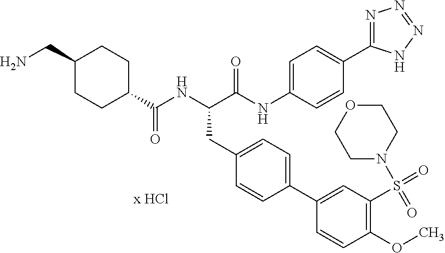 [SMILES: COc1ccc(-c2ccc(C[C@H](NC(=O)[C@H]3CC[C@H](CN)CC3)C(=O)Nc3ccc(-c4nnn[nH]4)cc3)cc2)cc1S(=O)(=O)N1CCOCC1]

268 μl (1.1 mmol) of 4M hydrogen chloride in dioxane were added to a solution of 86 mg (107 μmol) of tert-butyl [(trans-4-{[(2S)-3-[4′-methoxy-3′-(morpholin-4-ylsulphonyl)biphenyl-4-yl]-1-oxo-1-{[4-(1H-tetrazol-5-yl)phenyl]amino}propan-2-yl]carbamoyl}cyclohexyl)methyl]carbamate in 1 ml of dichloromethane. The mixture was stirred at RT for 3 d. Acetonitrile was added to the contents of the flask. The precipitated product was filtered off with suction, washed with acetonitrile and dried under high vacuum. The residue was purified by preparative HPLC (Method 8). 43 mg (53% of theory) of the title compound were obtained.

LC-MS (Method 4) Rₜ=0.83 min; MS (ESIpos): m/z=703 [M+H-HCl]⁺.

¹H-NMR (500 MHz, DMSO-d₆) δ ppm 0.84-0.98 (m, 2H), 1.13-1.32 (m, 2H), 1.39-1.49 (m, 1H), 1.56-1.64 (m, 1H), 1.68-1.79 (m, 3H), 2.10-2.19 (m, 1H), 2.63 (d, 2H), 2.85-2.94 (m, 1H), 3.05-3.13 (m, 5H), 3.56-3.61 (m, 4H), 3.93 (s, 3H), 4.66-4.75 (m, 1H), 7.34 (d, 1H), 7.40 (d, 2H), 7.56 (d, 2H), 7.60 (d, 2H), 7.84-7.94 (m, 4H), 8.13 (d, 1H), 10.14 (s, 1H).

### Example 25

### trans-4-(Aminomethyl)-N-{(2S)-3-[5′-(dimethylsulphamoyl)-2′-methylbiphenyl-4-yl]-1-oxo-1-[(2-oxo-2,3-dihydro-1H-benzimidazol-5-yl)amino]propan-2-yl}cyclohexanecarboxamide hydrochloride

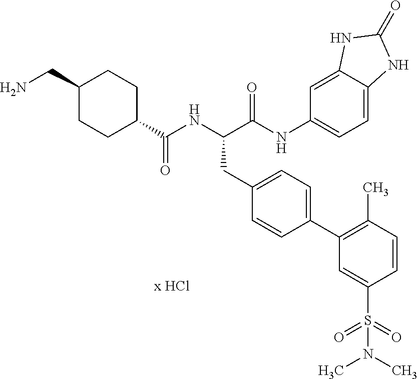 [SMILES: Cc1ccc(S(=O)(=O)N(C)C)cc1-c1ccc(C[C@H](NC(=O)[C@H]2CC[C@H](CN)CC2)C(=O)Nc2ccc3[nH]c(=O)[nH]c3c2)cc1]

114 μl (0.45 mmol) of 4M hydrogen chloride in dioxane were added to a solution of 66.7 mg (91 μmol) of tert-butyl {[trans-4-({(2S)-3-[5′-(dimethylsulphamoyl)-2′-methylbiphenyl-4-yl]-1-oxo-1-[(2-oxo-2,3-dihydro-1H-benzimidazol-5-yl)amino]propan-2-yl}carbamoyl)cyclohexyl]methyl}carbamate in 2 ml of dichloromethane. The mixture was stirred at RT for 4 days, and another 2.5 eq. of 4M hydrogen chloride in dioxane were added. The mixture was stirred at RT overnight. Acetonitrile was added to the contents of the flask. The precipitated product was filtered off with suction, washed with acetonitrile and dried under high vacuum. The residue was purified by preparative HPLC (Method 11). 14 mg (23% of theory) of the title compound were obtained.

LC-MS (Method 5) Rₜ=1.05 min; MS (ESIpos): m/z=633 [M+H-HCl]⁺.

¹H-NMR (400 MHz, DMSO-d₆) δ ppm 0.66-0.90 (m, 2H), 1.02-1.31 (m, 3H), 1.45-1.56 (m, 1H), 1.61-1.78 (m, 3H), 2.02-2.14 (m, 1H), 2.27 (s, 3H), 2.30-2.37 (m, 2H), 2.60 (s, 6H), 2.91 (dd, 1H), 3.08 (dd, 1H), 4.64-4.76 (m, 1H), 6.82 (d, 1H), 6.97 (dd, 1H), 7.29 (d, 2H), 7.37 (d, 2H), 7.40-7.44 (m, 2H), 7.55 (d, 1H), 7.63 (dd, 1H), 8.04 (d, 1H), 9.88-9.96 (m, 1H), 10.45 (br. s, 1H).

### Example 26

### trans-4-(Aminomethyl)-N-{(2S)-3-[2′-methyl-5′-(morpholin-4-ylsulphonyl)biphenyl-4-yl]-1-oxo-1-[(2-oxo-2,3-dihydro-1H-benzimidazol-5-yl)amino]propan-2-yl}cyclohexanecarboxamide hydrochloride

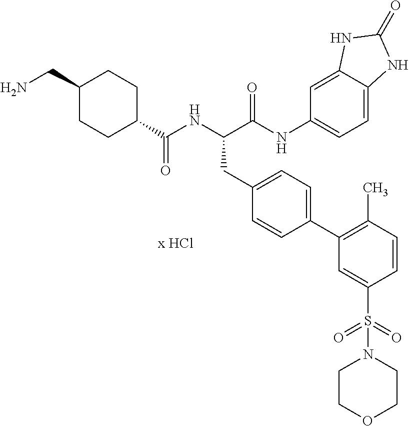 [SMILES: Cc1ccc(S(=O)(=O)N2CCOCC2)cc1-c1ccc(C[C@H](NC(=O)[C@H]2CC[C@H](CN)CC2)C(=O)Nc2ccc3[nH]c(=O)[nH]c3c2)cc1]

124 μl (0.50 mmol) of 4M hydrogen chloride in dioxane were added to a solution of 77 mg (99 μmol) of tert-butyl {[trans-4-({(2S)-3-[2′-methyl-5′-(morpholin-4-ylsulphonyl)biphenyl-4-yl]-1-oxo-1-[(2-oxo-2,3-dihydro-1H-benzimidazol-5-yl)amino]propan-2-yl}carbamoyl)cyclohexyl]methyl}carbamate in 2.5 ml of dichloromethane. The mixture was stirred at RT for 4 days. Acetonitrile was added to the contents of the flask. The precipitated product was filtered off with suction, washed with acetonitrile and dried under high vacuum. The residue was purified by preparative HPLC (Method 11). 14 mg (20% of theory) of the title compound were obtained.

LC-MS (Method 5) Rₜ=1.02 min; MS (ESIpos): m/z=675 [M+H-HCl]⁺.

¹H-NMR (400 MHz, DMSO-d₆) δ ppm 0.71-0.89 (m, 2H), 1.04-1.32 (m, 2H), 1.46-1.55 (m, 1H), 1.60-1.78 (m, 3H), 2.02-2.14 (m, 1H), 2.28 (s, 3H), 2.30-2.36 (m, 3H), 2.63-2.68 (m, 1H), 2.82-2.89 (m, 4H), 3.04-3.13 (m, 1H), 3.57-3.67 (m, 4H), 4.65-4.75 (m, 1H), 6.82 (d, 1H), 6.99 (dd, 1H), 7.29 (d, 2H), 7.35-7.45 (m, 4H), 7.54-7.65 (m, 2H), 8.04 (d, 1H), 9.91 (s, 1H), 10.47 (br. s, 1H).

### Example 27

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(morpholin-4-yl)ethyl]biphenyl-3-carboxamide hydrochloride

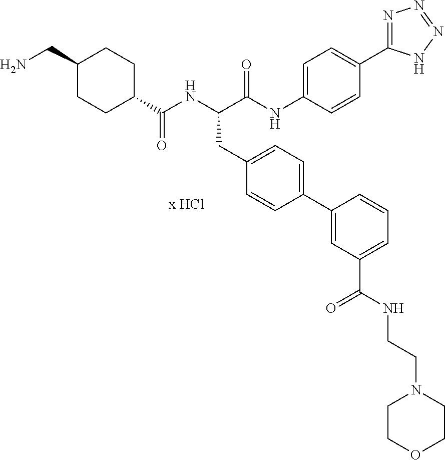 [SMILES: NC[C@H]1CC[C@H](C(=O)N[C@@H](Cc2ccc(-c3cccc(C(=O)NCCN4CCOCC4)c3)cc2)C(=O)Nc2ccc(-c3nnn[nH]3)cc2)CC1]

194 μl (0.78 mmol) of 4M hydrogen chloride in dioxane were added to a solution of 60.5 mg (78 μmol) of tert-butyl [(trans-4-{[(2S)-3-(3′-{[2-(morpholin-4-yl)ethyl]carbamoyl}biphenyl-4-yl)-1-oxo-1-{[4-(1H-tetrazol-5-yl)phenyl]amino}propan-2-yl]carbamoyl}cyclohexyl)methyl]carbamate formate in 3.3 ml of dioxane. The mixture was stirred at 35° C. overnight. Acetonitrile was added to the contents of the flask. The precipitated product was filtered off with suction, washed with acetonitrile and dried under high vacuum. 45 mg (80% of theory) of the title compound were obtained.

LC-MS (Method 4) Rₜ=0.69 min; MS (ESIpos): m/z=680 [M+H-HCl]⁺.

¹H-NMR (400 MHz, DMSO-d₆) δ ppm 0.81-0.98 (m, 2H), 1.09-1.33 (m, 2H), 1.38-1.51 (m, 1H), 1.54-1.63 (m, 1H), 1.66-1.81 (m, 3H), 2.07-2.17 (m, 1H), 2.55-2.62 (m, 2H), 2.88-2.98 (m, 1H), 3.06-3.14 (m, 2H), 3.52 (br. s, 2H), 3.66-3.73 (m, 2H), 3.80 (br. s, 2H), 3.94 (br. s, 2H), 4.65-4.74 (m, 1H), 7.41 (d, 2H), 7.52 (t, 1H), 7.67 (d, 2H), 7.74-7.88 (m, 7H), 7.99 (d, 2H), 8.18 (s, 1H), 8.24 (d, 1H), 8.96-9.05 (m, 1H), 10.55 (s, 1H).

### Example 28

### trans-4-(Aminomethyl)-N-[(2S)-3-[5′-(dimethylamino)-2′-methylbiphenyl-4-yl]-1-oxo-1-{[4-(1H-tetrazol-5-yl)phenyl]amino}propan-2-yl]cyclohexanecarboxamide hydrochloride

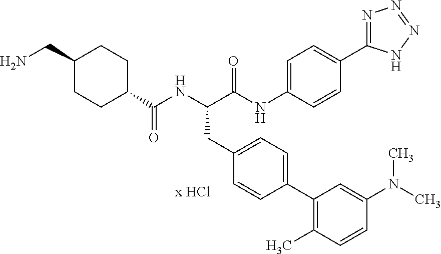 [SMILES: Cc1ccc(N(C)C)cc1-c1ccc(C[C@H](NC(=O)[C@H]2CC[C@H](CN)CC2)C(=O)Nc2ccc(-c3nnn[nH]3)cc2)cc1]

110 μl (0.44 mmol) of 4M hydrogen chloride in dioxane were added to a solution of 30 mg (44 μmol) of tert-butyl [(trans-4-{[(2S)-3-[5′-(dimethylamino)-2′-methylbiphenyl-4-yl]-1-oxo-1-{[4-(1H-tetrazol-5-yl)phenyl]amino}propan-2-yl]carbamoyl}cyclohexyl)methyl]carbamate formate in 1.8 ml of dioxane. The mixture was stirred at 35° C. overnight. Acetonitrile was added to the contents of the flask. The precipitated product was filtered off with suction, washed with acetonitrile and dried under high vacuum. 23 mg (78% of theory) of the title compound were obtained.

LC-MS (Method 4) Rₜ=0.74 min; MS (ESIpos): m/z=581 [M+H-HCl]⁺.

¹H-NMR (400 MHz, DMSO-d₆) δ ppm 0.80-0.96 (m, 2H), 1.07-1.31 (m, 2H), 1.38-1.59 (m, 2H), 1.65-1.80 (m, 3H), 2.08-2.18 (m, 4H), 2.54-2.66 (m, 2H), 2.89-2.97 (m, 1H), 3.02 (br. s., 6H), 3.08-3.16 (m, 1H), 4.66-4.79 (m, 1H), 7.19-7.43 (m, 6H), 7.74-7.91 (m, 5H), 8.00 (d, 2H), 8.25 (d, 1H), 10.54 (s, 1H).

### Example 29

### N-{4′-[(2S)-2-({[trans-4-(Aminomethyl)cyclohexyl]carbonyl}amino)-3-oxo-3-{[4-(2H-tetrazol-5-yl)phenyl]amino}propyl]-4-methylbiphenyl-3-yl}tetrahydro-2H-pyran-4-carboxamide hydrochloride

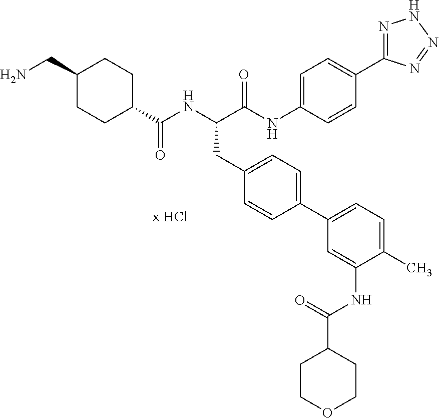 [SMILES: Cc1ccc(-c2ccc(C[C@H](NC(=O)[C@H]3CC[C@H](CN)CC3)C(=O)Nc3ccc(-c4nn[nH]n4)cc3)cc2)cc1NC(=O)C1CCOCC1]

96 μl (0.39 mmol) of 4M hydrogen chloride in dioxane were added to a solution of 22.6 mg (26 μmol) of tert-butyl [(trans-4-{[(2S)-3-{4′-methyl-3′-[(tetrahydro-2H-pyran-4-ylcarbonyl)amino]biphenyl-4-yl}-1-oxo-1-{[4-(2H-tetrazol-5-yl)phenyl]amino}propan-2-yl]carbamoyl}cyclohexyl)methyl]carbamate in 1 ml of dioxane. The mixture was stirred at RT for 5 days. The precipitated product was filtered off with suction, washed with dioxane and dried under high vacuum. 18 mg (95% of theory) of the title compound were obtained.

LC-MS (Method 1): Rₜ=0.69 min; MS (ESIpos): m/z=665 [M+H-HCl]⁺.

¹H-NMR (400 MHz, DMSO-d₆) δ ppm 0.84-0.99 (m, 2H), 1.09-1.34 (m, 2H), 1.38-1.53 (m, 1H), 1.56-1.82 (m, 7H), 2.10-2.18 (m, 1H), 2.21 (s, 3H), 2.58-2.76 (m, 3H), 2.88-2.99 (m, 1H), 3.06-3.14 (m, 1H), 3.32-3.42 (m, 2H), 3.57 (s, 3H), 3.92 (m, 2H), 4.72 (m, 1H), 7.27 (d, 1H), 7.34-7.42 (m, 3H), 7.53 (d, 2H), 7.65 (s, 1H), 7.76 (br. s., 3H), 7.83 (d, 2H), 8.02 (d, 2H), 8.24 (d, 1H), 9.34 (s, 1H), 10.55 (s, 1H).

### Example 30

### 4′-[(2S)-2-({[trans-4-(Aminomethyl)cyclohexyl]carbonyl}amino)-3-oxo-3-{[4-(2H-tetrazol-5-yl)phenyl]amino}propyl]-5-fluoro-N-isopropyl-6-methylbiphenyl-3-carboxamide hydrochloride

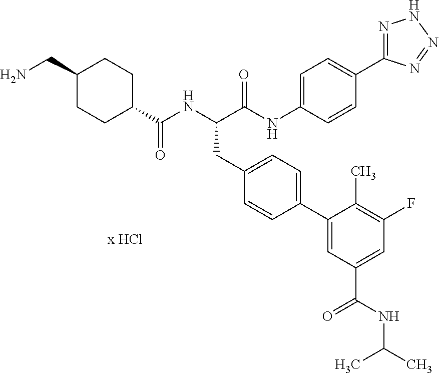 [SMILES: Cc1c(F)cc(C(=O)NC(C)C)cc1-c1ccc(C[C@H](NC(=O)[C@H]2CC[C@H](CN)CC2)C(=O)Nc2ccc(-c3nn[nH]n3)cc2)cc1]

0.45 ml (1.70 mmol) of 4M hydrogen chloride in dioxane was added to a suspension of 63 mg (0.085 mmol) of tert-butyl [(trans-4-{[(2S)-3-[3′-fluoro-5′-(isopropylcarbamoyl)-2′-methylbiphenyl-4-yl]-1-oxo-1-{[4-(2H-tetrazol-5-yl)phenyl]amino}propan-2-yl]carbamoyl}cyclohexyl)methyl]carbamate in 1.0 ml of dioxane. The reaction mixture was stirred at RT for another 1 h and allowed to stand at RT overnight. The mixture was then concentrated to dryness, a further 1.0 ml of 4M hydrogen chloride in dioxane was added to the residue and the mixture was stirred at RT for 3 h. The mixture was then concentrated to dryness and the residue was taken up in 3.0 ml of dioxane. The suspension obtained was filtered, and the solid was washed three times with diethyl ether and dried under high vacuum. 51 mg (85% of theory) of the title compound were obtained.

¹H-NMR (400 MHz, DMSO-d₆): δ=ppm 10.54 (br. s., 1H), 8.34-8.21 (m, 2H), 8.02 (d, 2H), 7.87-7.69 (m, 5H), 7.66-7.55 (m, 2H), 7.43 (d, 2H), 7.30 (d, 2H), 4.82-4.72 (m, 1H), 4.16-4.02 (m, 1H), 3.21-3.10 (m, 1H), 3.02-2.91 (m, 1H), 2.69-2.59 (m, 2H), 2.21-2.07 (m, 4H), 1.84-1.67 (m, 3H), 1.63-1.54 (m, 1H), 1.53-1.41 (m, 1H), 1.35-1.21 (m, 1H), 1.15 (d, 7H), 0.91 (d, 2H).

LC-MS (Method 1): Rₜ=0.76 min; MS (ESIpos): m/z=641 [M+H-HCl]⁺.

### Example 31

### 4′-[(2S)-2-({[trans-4-(Aminomethyl)cyclohexyl]carbonyl}amino)-3-oxo-3-{[4-(2H-tetrazol-5-yl)phenyl]amino}propyl]-5-fluoro-6-methyl-N-(piperidin-4-yl)biphenyl-3-carboxamide hydrochloride

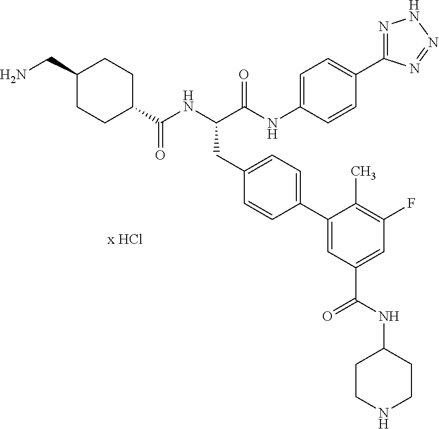 [SMILES: Cc1c(F)cc(C(=O)NC2CCNCC2)cc1-c1ccc(C[C@H](NC(=O)[C@H]2CC[C@H](CN)CC2)C(=O)Nc2ccc(-c3nn[nH]n3)cc2)cc1]

1.08 ml (4.31 mmol) of 4M hydrogen chloride in dioxane were added to a suspension of 95 mg (0.108 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-5-fluoro-6-methylbiphenyl-3-yl}carbonyl)amino]piperidine-1-carboxylate in 1.0 ml of dioxane, and the reaction mixture was stirred at RT for 45 min. The reaction mixture was then diluted with 3 ml of dioxane and filtered. The filter residue was washed three times with in each case 2 ml of diethyl ether, and the solid was dried under high vacuum. 75 mg (87% of theory) of the title compound were obtained.

¹H-NMR (400 MHz, DMSO-d₆): δ=ppm 16.79 (br. s., 1H), 10.54 (br. s., 1H), 8.75 (br. s., 2H), 8.55 (d, 1H), 8.28 (d, 1H), 8.02 (d, 2H), 7.88-7.74 (m, 5H), 7.66 (d, 1H), 7.59 (s, 1H), 7.43 (d, 2H), 7.30 (d, 2H), 4.82-4.71 (m, 1H), 4.11-3.99 (m, 1H), 3.20-3.10 (m, 1H), 3.06-2.91 (m, 3H), 2.69-2.59 (m, 2H), 2.21-2.06 (m, 4H), 2.00-1.89 (m, 2H), 1.82-1.67 (m, 6H), 1.63-1.54 (m, 1H), 1.53-1.41 (m, 1H), 1.41-1.05 (m, 3H), 1.01-0.84 (m, 2H).

LC-MS (Method 1): Rₜ=0.53 min; MS (ESIpos): m/z=682 [M+H-HCl]+.

### Example 32

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(trans-4-hydroxycyclohexyl)-6-methylbiphenyl-3-carboxamide hydrochloride

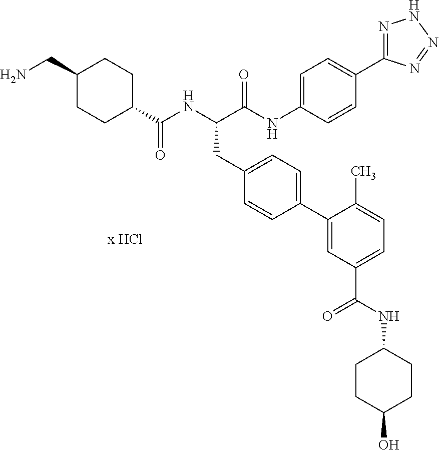 [SMILES: Cc1ccc(C(=O)N[C@H]2CC[C@H](O)CC2)cc1-c1ccc(C[C@H](NC(=O)[C@H]2CC[C@H](CN)CC2)C(=O)Nc2ccc(-c3nn[nH]n3)cc2)cc1]

0.32 ml (1.28 mmol) of 4M hydrogen chloride in dioxane was added to a suspension of 50 mg (0.064 mmol) of tert-butyl [(trans-4-{[(2S)-3-{5′-[(trans-4-hydroxycyclohexyl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate in 2.0 ml of dioxane, and the reaction mixture was allowed to stand in an ultrasonic bath at RT for 1 h. The reaction mixture was then concentrated and the residue was purified by preparative HPLC (mobile phase: acetonitrile/water with 0.1% formic acid (gradient)). 18 mg (36% of theory) of the title compound were obtained.

¹H-NMR (400 MHz, DMSO-d₆): δ=ppm 10.46 (s, 1H), 8.22 (d, 1H), 8.14 (d, 1H), 8.00 (d, 2H), 7.82 (d, 2H), 7.75-7.71 (m, 1H), 7.68-7.59 (m, 4H), 7.39 (d, 2H), 7.34 (d, 1H), 7.28 (d, 2H), 4.80-4.72 (m, 1H), 3.75-3.65 (m, 2H), 3.00-2.91 (m, 1H), 2.69-2.61 (m, 3H), 2.20 (s, 3H), 1.88-1.69 (m, 7H), 1.63-1.56 (m, 1H), 1.48-1.42 (m, 1H), 1.38-1.15 (m, 7H), 0.97-0.86 (m, 2H).

LC-MS (Method 1): Rₜ=0.69 min; MS (ESIpos): m/z=679 [M+H-HCl]⁺.

### Example 33

### trans-4-(Aminomethyl)-N-[(2S)-3-[2′-methyl-5′-(morpholin-4-ylcarbonyl)biphenyl-4-yl]-1-oxo-1-{[4-(2H-tetrazol-5-yl)phenyl]amino}propan-2-yl]cyclohexanecarboxamide hydrochloride

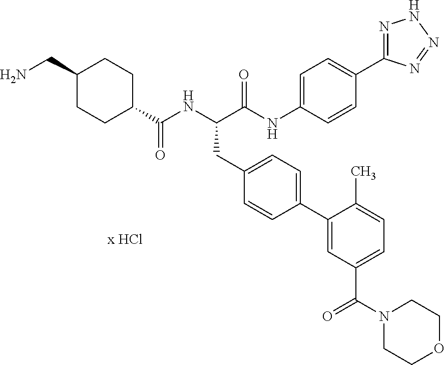 [SMILES: Cc1ccc(C(=O)N2CCOCC2)cc1-c1ccc(C[C@H](NC(=O)[C@H]2CC[C@H](CN)CC2)C(=O)Nc2ccc(-c3nn[nH]n3)cc2)cc1]

0.36 ml (1.43 mmol) of 4M hydrogen chloride in dioxane was added to a suspension of 54 mg (0.072 mmol) of tert-butyl [(trans-4-{[(2S)-3-[2′-methyl-5′-(morpholin-4-ylcarbonyl)biphenyl-4-yl]-1-oxo-1-{[4-(2H-tetrazol-5-yl)phenyl]amino}propan-2-yl]carbamoyl}cyclohexyl)methyl]carbamate in 2.6 ml of dioxane, and the reaction mixture was allowed to stand in an ultrasonic bath at RT for 1 h. Subsequently, the reaction mixture was concentrated and the residue was dried under high vacuum. 45 mg (88% of theory) of the title compound were obtained.

¹H-NMR (400 MHz, DMSO-d₆): δ=ppm 10.47 (s, 1H), 8.23 (d, 1H), 8.00 (d, 2H), 7.82 (d, 2H), 7.74-7.61 (m, 2H), 7.41-7.23 (m, 6H), 7.18-7.14 (m, 1H), 4.80-4.71 (m, 1H), 3.65-3.20 (m, 8H), 3.16-3.09 (m, 1H), 3.00-2.91 (m, 1H), 2.68-2.60 (m, 2H), 2.21 (s, 3H), 2.17-2.11 (m, 1H), 1.81-1.69 (m, 3H), 1.63-1.55 (m, 1H), 1.50-1.41 (m, 1H), 1.38-1.12 (m, 3H), 1.00-0.84 (m, 2H).

LC-MS (Method 1): Rₜ=0.68 min; MS (ESIpos): m/z=651 [M+H-HCl]⁺.

### Example 34

### 4′-[(2S)-2-({[trans-4-(Aminomethyl)cyclohexyl]carbonyl}amino)-3-oxo-3-{[4-(2H-tetrazol-5-yl)phenyl]amino}propyl]-5,6-dimethyl-N-(1-methylpiperidin-4-yl)biphenyl-3-carboxamide hydrochloride

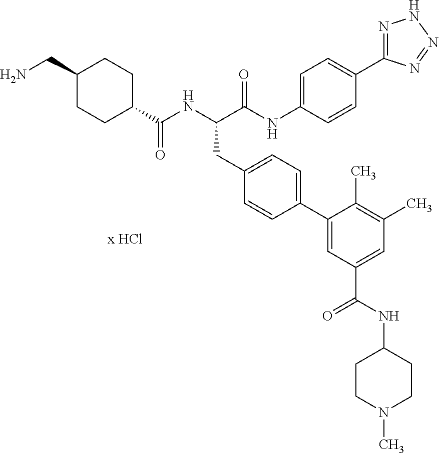 [SMILES: Cc1cc(C(=O)NC2CCN(C)CC2)cc(-c2ccc(C[C@H](NC(=O)[C@H]3CC[C@H](CN)CC3)C(=O)Nc3ccc(-c4nn[nH]n4)cc3)cc2)c1C]

0.35 ml (1.41 mmol) of 4M hydrogen chloride in dioxane was added to a suspension of 75 mg (0.094 mmol) of tert-butyl [(trans-4-{[(2S)-3-{2′,3′-dimethyl-5′-[(1-methylpiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]carbamate in 2.0 ml of dioxane, and the reaction mixture was stirred at RT overnight. The reaction mixture was then concentrated and the residue was purified by preparative HPLC (mobile phase: acetonitrile/water with 0.1% trifluoroacetic acid (gradient)). 34 mg (42% of theory) of the title compound were obtained.

¹H-NMR (400 MHz, DMSO-d₆): δ=ppm 16.73 (br. s., 1H), 10.48 (br. s., 1H), 9.87-9.71 (m, 1H), 8.44-8.37 (m, 1H), 8.28-8.22 (m, 1H), 8.01 (d, 2H), 7.82 (d, 2H), 7.79-7.63 (m, 3H), 7.51 (s, 1H), 7.39 (d, 2H), 7.22 (d, 2H), 4.80-4.70 (m, 1H), 4.05-3.93 (m, 1H), 3.49-3.38 (m, 2H), 3.18-3.02 (m, 3H), 3.00-2.90 (m, 1H), 2.74 (s, 3H), 2.68-2.61 (m, 2H), 2.33 (s, 3H), 2.21-2.13 (m, 1H), 2.08 (s, 3H), 2.02-1.93 (m, 2H), 1.87-1.69 (m, 5H), 1.65-1.56 (m, 1H), 1.53-1.42 (m, 1H), 1.33-1.13 (m, 2H), 0.99-0.85 (m, 2H).

LC-MS (Method 1): Rₜ=0.59 min; MS (ESIpos): m/z=692 [M+H-HCl]⁺.

### Example 35

### 4′-[(2S)-2-({[trans-4-(Aminomethyl)cyclohexyl]carbonyl}amino)-3-oxo-3-{[4-(2H-tetrazol-5-yl)phenyl]amino}propyl]-4-fluoro-N-(piperidin-4-yl)biphenyl-3-carboxamide hydrochloride

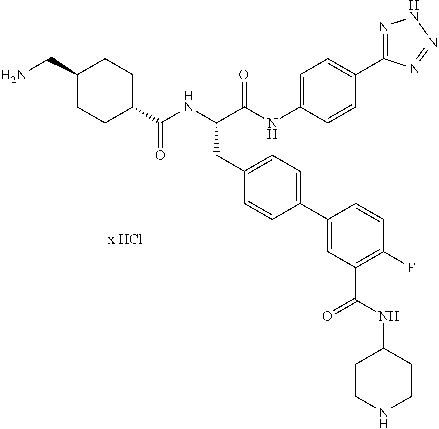 [SMILES: NC[C@H]1CC[C@H](C(=O)N[C@@H](Cc2ccc(-c3ccc(F)c(C(=O)NC4CCNCC4)c3)cc2)C(=O)Nc2ccc(-c3nn[nH]n3)cc2)CC1]

0.45 ml (1.79 mmol) of 4M hydrogen chloride in dioxane were added to a suspension of 104 mg (0.119 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-4-fluorobiphenyl-3-yl}carbonyl)amino]piperidine-1-carboxylate in 2.0 ml of dioxane, and the reaction mixture was allowed to stand at RT overnight. Subsequently, the reaction mixture was concentrated and the residue was triturated with diethyl ether. The solid present was filtered off and dried under high vacuum. 93 mg (94% of theory) of the title compound were obtained.

¹H-NMR (400 MHz, DMSO-d₆): δ=ppm 16.78 (br. s., 1H), 10.55 (br. s., 1H), 8.82-8.73 (m, 1H), 8.70-8.61 (m, 1H), 8.58 (d, 1H), 8.26 (d, 1H), 8.02 (d, 2H), 7.88-7.71 (m, 6H), 7.62 (d, 2H), 7.42 (d, 2H), 7.39-7.32 (m, 1H), 4.78-4.67 (m, 1H), 4.10-4.00 (m, 1H), 3.41-3.31 (m, 2H), 3.17-3.07 (m, 1H), 3.07-2.90 (m, 3H), 2.68-2.58 (m, 2H), 2.21-2.10 (m, 1H), 2.05-1.95 (m, 2H), 1.83-1.68 (m, 5H), 1.67-1.58 (m, 1H), 1.54-1.42 (m, 1H), 1.33-1.14 (m, 2H), 1.01-0.84 (m, 2H).

LC-MS (Method 1): Rₜ=0.51 min; MS (ESIpos): m/z=668 [M+H-HCl]⁺.

### Example 36

### 4′-[(2S)-2-({[trans-4-(Aminomethyl)cyclohexyl]carbonyl}amino)-3-oxo-3-{[4-(2H-tetrazol-5-yl)phenyl]amino}propyl]-6-methyl-N-(2-oxopiperidin-4-yl)biphenyl-3-carboxamide hydrochloride

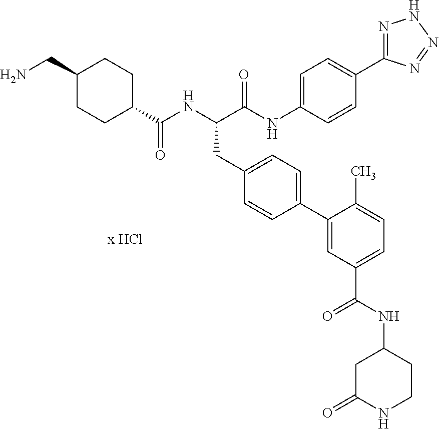 [SMILES: Cc1ccc(C(=O)NC2CCNC(=O)C2)cc1-c1ccc(C[C@H](NC(=O)[C@H]2CC[C@H](CN)CC2)C(=O)Nc2ccc(-c3nn[nH]n3)cc2)cc1]

1.5 ml (6.00 mmol) of 4M hydrogen chloride in dioxane was added to a suspension of 467 mg (0.60 mmol) of tert-butyl [(trans-4-{[(2S)-3-{2′-methyl-5′-[(2-oxopiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]carbamate in 24.4 ml of dioxane, and the reaction mixture was stirred at RT overnight. The reaction mixture was then concentrated and the residue was purified by preparative HPLC (mobile phase: acetonitrile/water with 0.1% trifluoroacetic acid (gradient)). 26 mg (5% of theory) of the title compound were obtained.

¹H-NMR (400 MHz, DMSO-d₆): δ=ppm 10.51 (br. s., 1H), 8.45-8.37 (m, 1H), 8.24 (d, 1H), 8.01 (d, 2H), 7.88-7.63 (m, 8H), 7.60-7.52 (m, 1H), 7.45-7.33 (m, 3H), 7.28 (d, 2H), 4.82-4.70 (m, 1H), 4.26-4.12 (m, 1H), 3.56-3.29 (m, 2H), 3.21-3.09 (m, 3H), 3.02-2.89 (m, 1H), 2.70-2.59 (m, 2H), 2.22 (s, 4H), 1.98-1.88 (m, 1H), 1.83-1.64 (m, 4H), 1.63-1.55 (m, 1H), 1.53-1.40 (m, 1H), 1.36-1.10 (m, 2H), 1.02-0.82 (m, 2H).

LC-MS (Method 1): Rₜ=0.67 min; MS (ESIpos): m/z=678 [M+H-HCl]⁺.

### Example 37

### 4′-[(2S)-24 {[trans-4-(Aminomethyl)cyclohexyl]carbonyl}amino)-3-oxo-3-{[4-(2H-tetrazol-5-yl)phenyl]amino}propyl]-N-[4-(dimethylamino)cyclohexyl]-5-methylbiphenyl-3-carboxamide hydrochloride

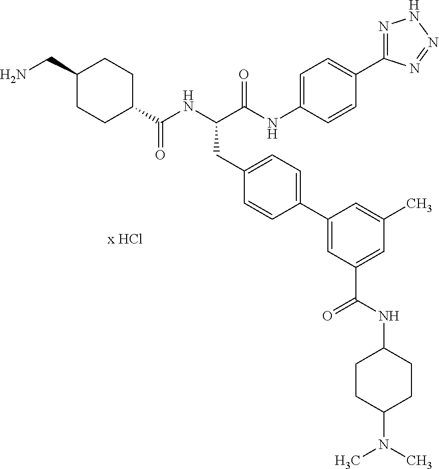 [SMILES: Cc1cc(C(=O)NC2CCC(N(C)C)CC2)cc(-c2ccc(C[C@H](NC(=O)[C@H]3CC[C@H](CN)CC3)C(=O)Nc3ccc(-c4nn[nH]n4)cc3)cc2)c1]

0.18 ml (0.71 mmol) of 4M hydrogen chloride in dioxane was added to a suspension of 28 mg (0.04 mmol) of tert-butyl [(trans-4-{[(2S)-3-(3′-{[4-(dimethylamino)cyclohexyl]carbamoyl}-5′-methylbiphenyl-4-yl)-1-oxo-1-{[4-(2H-tetrazol-5-yl)phenyl]amino}propan-2-yl]carbamoyl}cyclohexyl)methyl]carbamate in 1.0 ml of dioxane, and the reaction mixture was stirred at RT for 6 h and allowed to stand overnight. Subsequently, the mixture was concentrated, the residue was suspended in dioxane and another 1.0 ml of 4M hydrogen chloride in dioxane was added. The mixture was allowed to stand overnight. The mixture was then concentrated again, 1.0 ml of 4M hydrogen chloride in dioxane was added and the mixture was stirred in an ultrasonic bath for 1 h. The reaction mixture was concentrated, and the residue was stirred with diethyl ether and filtered. The filter residue was washed with diethyl ether and the solid was purified by preparative HPLC (mobile phase: acetonitrile/water with 0.1% trifluoroacetic acid (gradient)). 4 mg (14% of theory) of the title compound were obtained.

¹H-NMR (400 MHz, DMSO-d₆): δ=ppm 10.55-10.49 (m, 1H), 10.00-9.85 (m, 1H), 8.28-8.21 (m, 1H), 8.01 (d, 1H), 7.87-7.79 (m, 2H), 7.78-7.69 (m, 2H), 7.67-7.58 (m, 3H), 7.45-7.39 (m, 1H), 4.77-4.65 (m, 1H), 3.84-3.77 (m, 1H), 3.70-3.40 (m, 4H), 3.22-3.07 (m, 2H), 2.99-2.90 (m, 1H), 2.76-2.70 (m, 6H), 2.69-2.59 (m, 2H), 2.44-2.38 (m, 3H), 2.22-2.11 (m, 1H), 2.10-1.94 (m, 3H), 1.89-1.69 (m, 4H), 1.68-1.55 (m, 3H), 1.51-1.36 (m, 2H), 1.33-1.17 (m, 2H), 1.00-0.86 (m, 2H).

LC-MS (Method 1): Rₜ=0.57 min; MS (ESIpos): m/z=706 [M+H-HCl]⁺.

### Example 38

### trans-4-(Aminomethyl)-N-[(2S)-3-[4′-methyl-3′-(morpholin-4-ylcarbonyl)biphenyl-4-yl]-1-oxo-1-{[4-(2H-tetrazol-5-yl)phenyl]amino}propan-2-yl]cyclohexanecarboxamide hydrochloride

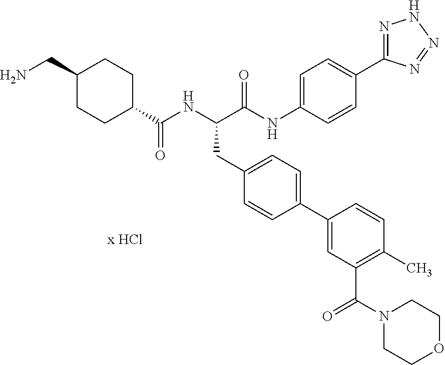 [SMILES: Cc1ccc(-c2ccc(C[C@H](NC(=O)[C@H]3CC[C@H](CN)CC3)C(=O)Nc3ccc(-c4nn[nH]n4)cc3)cc2)cc1C(=O)N1CCOCC1]

tert-Butyl [(trans-4-{[(2S)-3-[4′-methyl-3′-(morpholin-4-ylcarbonyl)biphenyl-4-yl]-1-oxo-1-{[4-(2H-tetrazol-5-yl)phenyl]amino}propan-2-yl]carbamoyl}cyclohexyl)methyl]carbamate (55 mg, 0.07 mmol) was initially charged in 2.0 ml of dioxane, 0.18 ml (0.73 mmol) of 4M hydrogen chloride in dioxane was added and the mixture was stirred for 3 days. The mixture was then stirred in an ultrasonic bath for 2 h. The suspension obtained was filtered, and the filter residue was washed with dioxane and dried under high vacuum. 31 mg (60% of theory) of the title compound were obtained.

¹H-NMR (400 MHz, DMSO-d₆): δ=ppm 10.52 (s, 1H), 8.22 (d, 1H), 8.01 (d, 2H), 7.83 (d, 2H), 7.72 (br. s., 3H), 7.64-7.57 (m, 3H), 7.48-7.44 (m, 1H), 7.39 (d, 2H), 7.34 (d, 1H), 4.77-4.66 (m, 1H), 3.75-3.59 (m, 4H), 3.56-3.51 (m, 1H), 3.50-3.45 (m, 1H), 3.22-3.06 (m, 4H), 2.98-2.88 (m, 1H), 2.63 (d, 2H), 2.25 (s, 3H), 2.20-2.10 (m, 1H), 1.81-1.68 (m, 3H), 1.66-1.57 (m, 1H), 1.51-1.40 (m, 1H), 1.31-1.13 (m, 2H), 0.99-0.84 (m, 2H).

LC-MS (Method 1): Rₜ=0.70 min; MS (ESIpos): m/z=651 [M+H-HCl]⁺.

### Example 39

### 4′-[(2S)-2-({[trans-4-(Aminomethyl)cyclohexyl]carbonyl}amino)-3-oxo-3-{[4-(2H-tetrazol-5-yl)phenyl]amino}propyl]-N,N,4,6-tetramethylbiphenyl-3-carboxamide hydrochloride

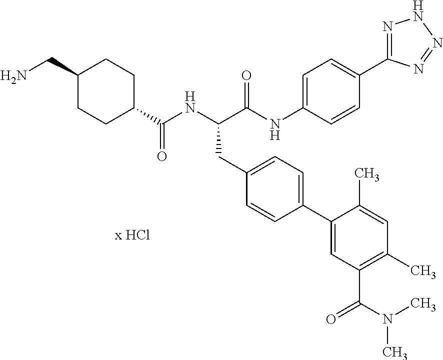 [SMILES: Cc1cc(C)c(-c2ccc(C[C@H](NC(=O)[C@H]3CC[C@H](CN)CC3)C(=O)Nc3ccc(-c4nn[nH]n4)cc3)cc2)cc1C(=O)N(C)C]

tert-Butyl [(trans-4-{[(2S)-3-[5′-(dimethylcarbamoyl)-2′,4′-dimethylbiphenyl-4-yl]-1-oxo-1-[4-(2H-tetrazol-5-yl)phenyl]amino propan-2-yl]carbamoyl}cyclohexyl)methyl]carbamate (73 mg, 0.10 mmol) was initially charged in 2.2 ml of dioxane, 0.50 ml (2.01 mmol) of 4M hydrogen chloride in dioxane was added and the mixture was allowed to stand overnight. The reaction mixture was then concentrated completely, the residue was triturated with diethyl ether and filtered and the filter residue was dried under high vacuum. 61 mg (83% of theory) of the title compound were obtained.

¹H-NMR (400 MHz, DMSO-d₆): δ=ppm 10.45 (s, 1H), 8.21 (d, 1H), 7.99 (d, 2H), 7.81 (d, 2H), 7.73-7.62 (m, 3H), 7.36 (d, 2H), 7.23 (d, 2H), 7.16 (s, 1H), 6.89 (s, 1H), 4.80-4.69 (m, 1H), 3.16-3.07 (m, 1H), 2.98 (s, 3H), 2.95-2.90 (m, 1H), 2.78 (s, 3H), 2.69-2.60 (m, 2H), 2.23-2.08 (m, 7H), 1.82-1.67 (m, 2H), 1.63-1.54 (m, 1H), 1.53-1.38 (m, 1H), 1.32-1.21 (m, 2H), 1.20-1.13 (m, 1H), 1.00-0.83 (m, 2H).

LC-MS (Method 1): Rₜ=1.17 min; MS (ESIpos): m/z=868 [M+H-HCl]⁺.

### Example 40

### 4′-[(2S)-2-({[trans-4-(Aminomethyl)cyclohexyl]carbonyl}amino)-3-oxo-3-{[4-(2H-tetrazol-5-yl)phenyl]amino}propyl]-5-methyl-N-(piperidin-4-yl)biphenyl-3-carboxamide hydrochloride

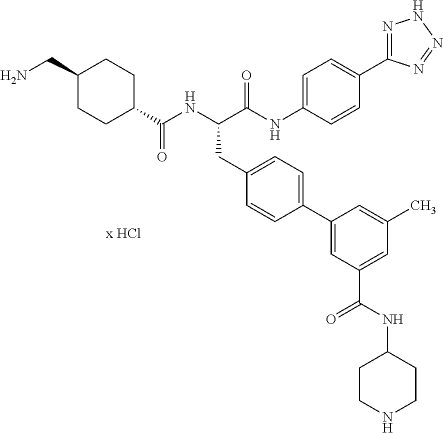 [SMILES: Cc1cc(C(=O)NC2CCNCC2)cc(-c2ccc(C[C@H](NC(=O)[C@H]3CC[C@H](CN)CC3)C(=O)Nc3ccc(-c4nn[nH]n4)cc3)cc2)c1]

tert-Butyl [(trans-4-{[(2S)-3-[3′-methyl-5′-(piperidin-4-ylcarbamoyl)biphenyl-4-yl]-1-oxo-1-{[4-(2H-tetrazol-5-yl)phenyl]amino}propan-2-yl]carbamoyl}cyclohexyl)methyl]carbamate (54 mg, 0.06 mmol) was dissolved in methanol/dioxane, 0.31 ml of 4M hydrogen chloride in dioxane was added and the mixture was stirred in an ultrasonic bath for 30 min and allowed to stand at RT for 1 h. Subsequently, the reaction mixture was concentrated fully and the residue was dried under high vacuum. 45 mg (92% of theory) of the title compound were obtained.

¹H-NMR (400 MHz, DMSO-d₆): δ=ppm 16.87 (br. s., 1H), 10.58 (br. s., 1H), 8.85-8.68 (m, 2H), 8.54 (d, 1H), 8.29 (d, 1H), 8.02 (d, 2H), 7.91-7.74 (m, 6H), 7.69-7.58 (m, 4H), 7.43 (d, 2H), 4.76-4.68 (m, 1H), 4.14-4.01 (m, 1H), 3.17-3.09 (m, 1H), 3.08-2.90 (m, 3H), 2.69-2.57 (m, 2H), 2.41 (s, 3H), 2.22-2.11 (m, 1H), 2.02-1.92 (m, 2H), 1.84-1.70 (m, 6H), 1.67-1.57 (m, 1H), 1.53-1.42 (m, 1H), 1.34-1.12 (m, 3H), 1.00-0.83 (m, 2H).

LC-MS (Method 1): Rₜ=0.58 min; MS (ESIpos): m/z=664 [M+H-HCl]⁺.

### Example 41

### 3-{5-[4-({(2S)-2-({[trans-4-(Aminomethyl)cyclohexyl]carbonyl}amino)-3-[2′-methyl-5′-(methylcarbamoyl)biphenyl-4-yl]propanoyl}amino)phenyl]-4H-1,2,4-triazol-3-yl}-2,2,3,3-tetrafluoropropanoic acid hydrochloride

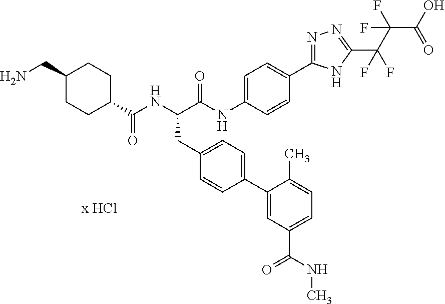 [SMILES: CNC(=O)c1ccc(C)c(-c2ccc(C[C@H](NC(=O)[C@H]3CC[C@H](CN)CC3)C(=O)Nc3ccc(-c4nnc(C(F)(F)C(F)(F)C(=O)O)[nH]4)cc3)cc2)c1]

3-{5-[4-({(2S)-2-{[(trans-4-{[(tert-Butoxycarbonyl)amino]methyl}cyclohexyl)carbonyl]amino}-3-[2′-methyl-5′-(methylcarbamoyl)biphenyl-4-yl]propanoyl}amino)phenyl]-4H-1,2,4-triazol-3-yl}-2,2,3,3-tetrafluoropropanoic acid (9.2 mg, 0.01 mmol) was initially charged in 1.0 ml of dioxane, and 14 μl of 4M hydrogen chloride in dioxane were added. 0.25 ml of methanol was then added, and the mixture was stirred in an ultrasonic bath at RT for 30 min. The reaction mixture was allowed to stand at RT overnight and concentrated, 0.5 ml of 4M hydrogen chloride in dioxane was added and the mixture was stirred at RT in the ultrasonic bath for a further 30 min. The reaction mixture was subsequently concentrated, and the residue was taken up in water/acetonitrile and purified twice by preparative HPLC (mobile phase: acetonitrile/water with 0.1% trifluoroacetic acid (gradient) and acetonitrile/water with 0.1% aqueous ammonia solution (gradient)). This gave 1.1 mg (13% of theory) of the title compound.

¹H-NMR (400 MHz, DMSO-d₆): δ=ppm 10.40 (br. s., 1H), 8.44-8.38 (m, 1H), 8.28-8.21 (m, 1H), 7.95 (d, 2H), 7.81-7.63 (m, 5H), 7.43-7.32 (m, 3H), 7.28 (d, 2H), 4.79-4.69 (m, 1H), 3.50-3.30 (m, 1H), 3.19-3.10 (m, 1H), 2.99-2.91 (m, 1H), 2.76 (d, 3H), 2.67-2.60 (m, 2H), 2.22 (s, 3H), 2.20-2.12 (m, 1H), 1.80-1.69 (m, 3H), 1.63-1.55 (m, 1H), 1.51-1.41 (m, 1H), 1.33-1.12 (m, 3H), 0.98-0.85 (m, 2H)

LC-MS (Method 1): Rₜ=0.99 min; MS (ESIpos): m/z=751 [M+H-HCl]⁺.

### Example 42

### 3-[5-(4-{[(2S)-2-({[trans-4-(Aminomethyl)cyclohexyl]carbonyl}amino)-3-{2′-methyl-5′-[(1-methylpiperidin-4-yl)carbamoyl]biphenyl-4-yl}propanoyl]amino}phenyl)-1H-1,2,4-triazol-3-yl]-2,2,3,3-tetrafluoropropanoic acid hydrochloride

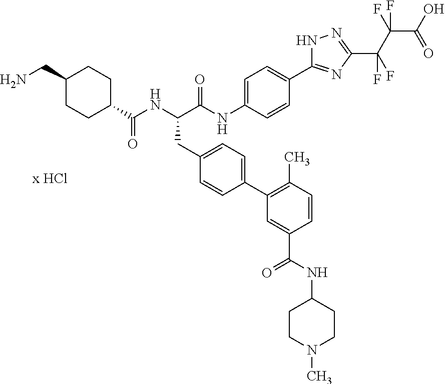 [SMILES: Cc1ccc(C(=O)NC2CCN(C)CC2)cc1-c1ccc(C[C@H](NC(=O)[C@H]2CC[C@H](CN)CC2)C(=O)Nc2ccc(-c3nc(C(F)(F)C(F)(F)C(=O)O)n[nH]3)cc2)cc1]

4M hydrogen chloride in 1,4-dioxane (0.48 ml, 1.91 mmol) was added to a solution of 3-[5-(4-{[(2S)-2-{[(trans-4-{[(tert-butoxycarbonyl)amino]methyl}cyclohexyl)carbonyl]amino}-3-{2′-methyl-5′-[(1-methylpiperidin-4-yl)carbamoyl]biphenyl-4-yl}propanoyl]amino}phenyl)-1H-1,2,4-triazol-3-yl]-2,2,3,3-tetrafluoropropanoic acid (117 mg, 0.13 mmol) in dioxane (4 ml), and the mixture was stirred at RT overnight. The solid was filtered off with suction, washed with dioxane and then dried under high vacuum. This gave 113 mg (99% of theory) of the title compound.

¹H-NMR (400 MHz, DMSO-d₆): δ=ppm 0.81-1.01 (m, 2H), 1.12-1.35 (m, 2H), 1.41-1.52 (m, 1H), 1.54-1.64 (m, 1H), 1.66-1.91 (m, 4H), 1.92-2.02 (m, 2H), 2.09-2.18 (m, 1H), 2.21 (s, 3H), 2.59-2.67 (m, 2H), 2.72 (s, 3H), 2.91-3.00 (m, 1H), 3.01-3.19 (m, 3H), 3.34-3.47 (m, 3H), 3.94-4.07 (m, 1H), 4.68-4.82 (m, 1H), 7.28 (d, 2H), 7.33-7.38 (m, 1H), 7.39-7.45 (m, 2H), 7.68 (s, 1H), 7.72-7.88 (m, 5H), 7.98 (d, 2H), 8.29 (d, 1H), 8.50 (d, 1H), 9.91-10.27 (m, 1H), 10.42-10.64 (m, 1H), 14.96-15.33 (m, 1H).

LC-MS (Method 1): Rₜ=0.56 min; MS (ESIneg): m/z=819 [M-HCl]⁻.

### Example 43

### 3-{5-[4-({(2S)-2-({[trans-4-(Aminomethyl)cyclohexyl]carbonyl}amino)-3-[5′-(isopropylcarbamoyl)-2′-methylbiphenyl-4-yl]propanoyl}amino)phenyl]-1H-1,2,4-triazol-3-yl}propanoic acid hydrochloride

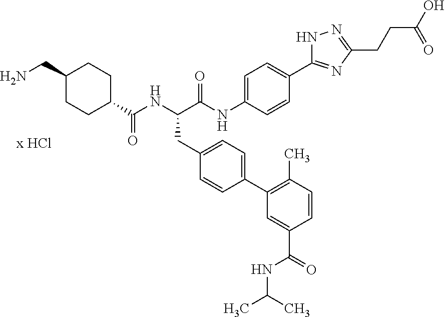 [SMILES: Cc1ccc(C(=O)NC(C)C)cc1-c1ccc(C[C@H](NC(=O)[C@H]2CC[C@H](CN)CC2)C(=O)Nc2ccc(-c3nc(CCC(=O)O)n[nH]3)cc2)cc1]

4M hydrogen chloride in 1,4-dioxane (0.19 ml, 0.75 mmol) was added to a solution of tert-butyl 3-{5-[4-({(2S)-2-{[(trans-4-{[(tert-butoxycarbonyl)amino]methyl}cyclohexyl)carbonyl]amino}-3-[5′-(isopropylcarbamoyl)-2′-methylbiphenyl-4-yl]propanoyl}amino)phenyl]-1H-1,2,4-triazol-3-yl}propanoate (42.2 mg, 0.05 mmol) in dioxane (1.5 ml), and the mixture was stirred at RT for three days. The solid was filtered off with suction, washed with dioxane and then dried under high vacuum. This gave 37.4 mg (96% of theory) of the title compound.

¹H-NMR (400 MHz, DMSO-d₆): δ=ppm 0.84-1.01 (m, 2H), 1.14 (s, 3H), 1.15 (s, 3H), 1.17-1.34 (m, 2H), 1.41-1.53 (m, 1H), 1.53-1.63 (m, 1H), 1.74 (d, 3H), 2.10-2.19 (m, 1H), 2.22 (s, 3H), 2.63 (m, 2H), 2.74-2.83 (m, 2H), 2.91-3.04 (m, 3H), 3.10-3.19 (m, 1H), 4.08 (m, 1H), 4.70-4.79 (m, 1H), 7.29 (d, 2H), 7.35 (d, 1H), 7.41 (d, 2H), 7.68 (d, 1H), 7.71-7.79 (m, 3H), 7.84 (br. s., 3H), 7.97 (d, 2H), 8.22 (d, 1H), 8.28 (d, 1H), 10.48 (br. s., 1H).

LC-MS (Method 1): Rₜ=0.73 min; MS (ESIneg): m/z=692 [M-HCl]⁻.

### Example 44

### 3-{5-[4-({(2S)-2-({[trans-4-(Aminomethyl)cyclohexyl]carbonyl}amino)-3-[4′-methoxy-3′-(morpholin-4-ylsulphonyl)biphenyl-4-yl]propanoyl}amino)phenyl]-1H-1,2,4-triazol-3-yl}-2,2,3,3-tetrafluoropropanoic acid hydrochloride

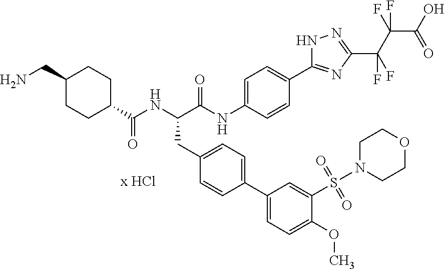 [SMILES: COc1ccc(-c2ccc(C[C@H](NC(=O)[C@H]3CC[C@H](CN)CC3)C(=O)Nc3ccc(-c4nc(C(F)(F)C(F)(F)C(=O)O)n[nH]4)cc3)cc2)cc1S(=O)(=O)N1CCOCC1]

364 μl (1.5 mmol) of 4M hydrogen chloride in dioxane were added to a solution of 92 mg (97 μmol) of 3-{5-[4-({(2S)-2-{[(trans-4-{[(tert-butoxycarbonyl)amino]-methyl}cyclohexyl)carbonyl]amino}-3-[4′-methoxy-3′-(morpholin-4-ylsulphonyl)biphenyl-4-yl]propanoyl}amino)phenyl]-1H-1,2,4-triazol-3-yl}-2,2,3,3-tetrafluoropropanoic acid in 2 ml of dioxane. The mixture was then stirred at RT for 18 h. Acetonitrile was added and the solid obtained was filtered, washed with acetonitrile and dried under high vacuum. 76 mg (86% of theory) of the title compound were obtained.

¹H-NMR (400 MHz, DMSO-d₆): δ=ppm 0.78-1.01 (m, 2H), 1.12-1.36 (m, 2H), 1.41-1.65 (m, 2H), 1.75 (m, 3H), 2.08-2.22 (m, 1H), 2.63 (m, 2H), 2.87-3.02 (m, 1H), 3.06-3.16 (m, 5H), 3.53-3.64 (m, 4H), 3.94 (s, 3H), 4.65-4.79 (m, 1H), 7.36 (d, 1H), 7.42 (d, 2H), 7.58 (d, 2H), 7.82 (m, 4H), 7.90-7.96 (m, 2H), 8.00 (d, 2H), 8.26 (d, 1H), 10.58 (br. s., 1H), 15.20 (br. s, 1H).

LC-MS (Method 1): Rₜ=0.69 min; MS (ESIpos): m/z=846.4 [M+H-HCl]⁺.

### Example 45

### trans-4-(Aminomethyl)-N-{(2S)-3-[2′-methyl-5′-(pyrrolidin-1-ylsulphonyl)biphenyl-4-yl]-1-oxo-1-[(3-oxo-2,3-dihydro-1H-indazol-6-yl)amino]propan-2-yl}cyclohexanecarboxamide hydrochloride

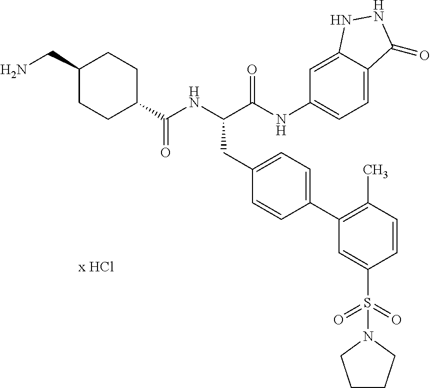 [SMILES: Cc1ccc(S(=O)(=O)N2CCCC2)cc1-c1ccc(C[C@H](NC(=O)[C@H]2CC[C@H](CN)CC2)C(=O)Nc2ccc3c(=O)[nH][nH]c3c2)cc1]

4M hydrogen chloride in dioxane is added to a solution of tert-butyl {[trans-4-({(2S)-3-[2′-methyl-5′-(pyrrolidin-1-ylsulphonyl)biphenyl-4-yl]-1-oxo-1-[(3-oxo-2,3-dihydro-1H-indazol-6-yl)amino]propan-2-yl}carbamoyl)cyclohexyl]methyl}carbamate in dioxane. The mixture is stirred at RT for 3 to 4 days The reaction mixture is worked up by methods known to those skilled in the art and the residue is separated by means of preparative HPLC. This gives the title compound.

### Example 46

### trans-4-(Aminomethyl)-N-{(2S)-3-[5′-(dimethylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

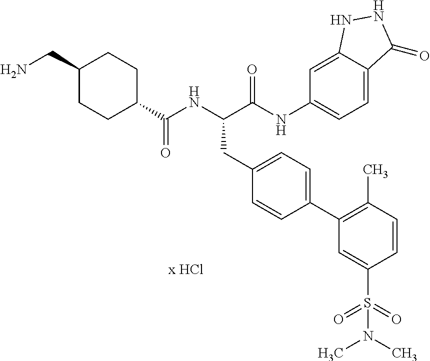 [SMILES: Cc1ccc(S(=O)(=O)N(C)C)cc1-c1ccc(C[C@H](NC(=O)[C@H]2CC[C@H](CN)CC2)C(=O)Nc2ccc3c(=O)[nH][nH]c3c2)cc1]

4M hydrogen chloride in dioxane is added to a solution of tert-butyl {[trans-4-({(2S)-3-[5′-(dimethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in 2 ml of dichloromethane. The mixture is stirred at RT for 3 to 4 days. The reaction mixture is worked up by methods known to those skilled in the art and the residue is separated by means of preparative HPLC. This gives the title compound.

### Example 47

### trans-4-(Aminomethyl)-N-{(2S)-3-[2′-methyl-5′-(morpholin-4-ylsulphonyl)biphenyl-4-yl]-1-oxo-1-[(3-oxo-2,3-dihydro-1H-indazol-6-yl)amino]propan-2-yl}cyclohexanecarboxamide hydrochloride

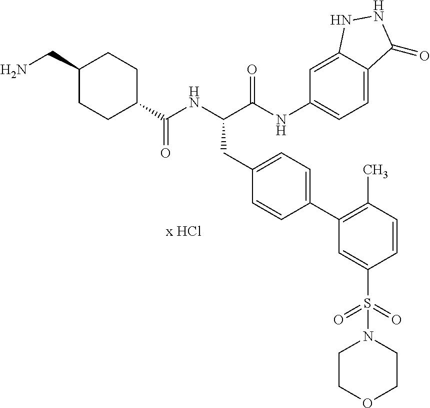 [SMILES: Cc1ccc(S(=O)(=O)N2CCOCC2)cc1-c1ccc(C[C@H](NC(=O)[C@H]2CC[C@H](CN)CC2)C(=O)Nc2ccc3c(=O)[nH][nH]c3c2)cc1]

4M hydrogen chloride in dioxane is added to a solution of tert-butyl {[trans-4-({(2S)-3-[2′-methyl-5′-(morpholin-4-ylsulphonyl)biphenyl-4-yl]-1-oxo-1-[(3-oxo-2,3-dihydro-1H-indazol-6-yl)amino]propan-2-yl}carbamoyl)cyclohexyl]methyl}carbamate in dichloromethane. The mixture is stirred at RT for 3 to 4 days The reaction mixture is worked up by methods known to those skilled in the art and the residue is separated by means of preparative HPLC. This gives the title compound.

### B) ASSESSMENT OF PHYSIOLOGICAL EFFICACY

The suitability of the compounds according to the invention for treating thromboembolic or hyperfibrinolytic disorders can be demonstrated in the following assay systems:

### a) Test Descriptions (In Vitro)

### a.1) Measurement of FXIa Inhibition

The factor XIa inhibition of the substances according to the invention is determined using a biochemical test system which utilizes the reaction of a peptidic factor XIa substrate to determine the enzymatic activity of human factor XIa. Here, factor XIa cleaves from the peptic factor XIa substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). Subsequently, the following are added successively: 20 μl of assay buffer (50 mmol/l Tris buffer pH 7.4; 100 mmol/l sodium chloride; 5 mmol/l calcium chloride; 0.1% bovine serum albumin) and 20 μl of factor XIa from Kordia (0.45 nM in assay buffer). After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulphoxide instead of test substance in dimethyl sulphoxide), and IC₅₀ values are calculated from the concentration/activity relationships. Activity data from this test are listed in Table A below:

TABLE A

| Example No. | IC₅₀ [nM] | Example No. | IC₅₀ [nM] |
|---|---|---|---|
| 1 | 0.5 | 2 | 0.1 |
| 3 | 2.8 | 4 | 3.1 |
| 5 | 11.0 | 6 | 14.0 |
| 7 | 11.0 | 8 | 6.9 |
| 9 | 7.4 | 10 | 8.0 |
| 11 | 6.9 | 12 | 8.1 |
| 13 | 17.0 | 14 | 15.0 |
| 15 | 10.0 | 16 | 9.0 |
| 17 | 9.3 | 18 | 16.0 |
| 19 | 6.4 | 20 | 2.7 |
| 21 | 8.3 | 22 | 25.0 |
| 23 | 13.0 | 24 | 2.0 |
| 25 | 6.1 | 26 | 6.1 |
| 27 | 6.0 | 28 | 1.5 |
| 29 | 11.0 | 30 | 2.7 |
| 31 | 4.5 | 32 | 4.7 |
| 33 | 5.4 | 34 | 4.1 |
| 35 | 10 | 36 | 1.9 |
| 37 | 7.5 | 38 | 4.1 |
| 39 | 3.3 | 40 | 4.1 |
| 41 | 2.8 | 42 | 6.8 |
| 43 | 9.7 | 44 | 2.2 |

### a.2) Determination of the Selectivity

To demonstrate the selectivity of the substances with respect to FXIa inhibition, the test substances are examined for their inhibition of other human serin proteases, such as factor Xa, trypsin and plasmin. To determine the enzymatic activity of factor Xa (1.3 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of sodium chloride, 0.1% BSA [bovine serum albumin], 5 mmol/l of calcium chloride, pH 7.4) and incubated for 15 min with test substance in various concentrations in dimethyl sulphoxide and also with dimethyl sulphoxide without test substance. The enzymatic reaction is then started by addition of the appropriate substrates (5 μmol/l of Boc-Ile-Glu-Gly-Arg-AMC from Bachem for factor Xa and trypsin, 50 μmol/l of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulphoxide instead of test substance in dimethyl sulphoxide) and IC₅₀ values are calculated from the concentration/activity relationships.

### a.3) Thrombin Generation Assay (Thrombogram)

The effect of the test substances on the thrombogram (thrombin generation assay according to Hemker) is determined in vitro in human plasma (Octaplas® from Octapharma).

In the thrombin generation assay according to Hemker, the activity of thrombin in coagulating plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 pM or 0.1 pM recombinant tissue factor, 24 μM phospholipids in HEPES) are used. In addition, a thrombin calibrator from Thrombinoscope is used whose amidolytic activity is required for calculating the thrombin activity in a sample containing an unknown amount of thrombin. The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM HEPES, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

### a.4) Determination of Anticoagulatory Activity

The anticoagulatory activity of the test substances is determined in vitro in human and animal plasma (for example mouse, rat, rabbit, pig and dog plasma). To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

The activated partial thromboplastin time (aPTT) is determined in the presence of varying concentrations of test substance or the corresponding solvent using a commercial test kit (C.K. Prest from Diagnostica Stago). The test compounds are incubated with the plasma and the PTT reagent (cephalin, kaolin) at 37° C. for 3 minutes. Coagulation is then started by addition of a 25 mM aqueous calcium chloride solution, and the time when coagulation occurs is determined. The concentration of test substance which brings about a 1.5-fold extension of the aPTT is determined Activity data from this test are listed in Table B below:

TABLE B

| | aPTT | | |
|---|---|---|---|
| Example No. | [μmol/l] | Example No. | aPTT [μmol/l] |
| 1 | 0.16 | 2 | 0.22 |
| 3 | 0.19 | 4 | 0.16 |
| 5 | 0.37 | 6 | 0.54 |
| 7 | 0.39 | 8 | 0.51 |
| 9 | 0.08 | 10 | 0.38 |
| 11 | 0.26 | 12 | 0.49 |
| 13 | 0.28 | 14 | 0.35 |
| 15 | 0.52 | 16 | 0.2 |
| 17 | 0.58 | 18 | 0.35 |
| 19 | 0.26 | 20 | 0.39 |
| 21 | 0.22 | 22 | 0.34 |
| 23 | 0.36 | 24 | 0.09 |
| 25 | 0.15 | 26 | 0.18 |
| 27 | 0.19 | 28 | 0.26 |
| 29 | 0.3 | 30 | 0.2 |
| 31 | 0.16 | 32 | 0.15 |
| 33 | 0.18 | 34 | 0.06 |
| 35 | 0.2 | 36 | 0.14 |
| 37 | 0.14 | 38 | 0.22 |
| 39 | 0.17 | 40 | 0.06 |
| 41 | 0.31 | 42 | 0.16 |
| 43 | 0.38 | 44 | 0.2 |

### a.5) Determination of Fibrinolytic Activity

Antifibrinolytic activity in vitro is assessed in human, platelet-free plasma. Tissue factor (TF) (1 pM) and tissue plasminogen activator (tPA) (40 nM) are pipetted into plasma together with 12.5 mM aqueous calcium chloride solution and substance. On occurrence of clotting, the subsequent clot lysis is determined photometrically over a period of 30 minutes.

### b) Determination of Antithrombotic Activity (In Vivo)

b.1) Arterial Thrombosis Model (Iron(II) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined.

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is furthermore maintained by intravenous administration of the same preparations (bolus: continuous infusion) via the right auricular vein.

The right carotid artery is exposed and the vessel injury is then caused by wrapping a piece of filter paper (10 mm×10 mm) on a Parafilm® strip (25 mm×12 mm) around the carotid artery without disturbing the blood flow. The filter paper contains 100 μL of a 13% strength solution of iron(II) chloride (Sigma) in water. After 5 min, the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3-mm-long incision (blade Art. Number 10-150-10, Martin, Tuttlingen, Germany) is made parallel to the longitudinal axis of the ear. Care is taken here not to damage any visible vessels. Any blood that extravasates is taken up in 15 second intervals using accurately weighed filter paper pieces, without touching the wound directly. Bleeding time is calculated as the time from making the incision to the point in time where no more blood can be detected on the filter paper. The volume of the extravasated blood is calculated after weighing of the filter paper pieces.

### c) Determination of Fibrinolytic Activity (In Vivo)

### c.1) Hyper-Fibrinolytic Rats

The determination of antifibrinolytic activity in vivo is conducted in hyperfibrinolytic rats. After anaesthetization and catheterization of the animals, hyperfibrinolysis is triggered by infusion of tissue plasminogen activator (tPA) (8 mg/kg/h). 10 minutes after commencement of tPA infusion, the substances are administered as an i.v. bolus. After a further 15 minutes, tPA infusion is ended and a transsection of the tail is conducted. Subaqual bleeding (in physiological saline at 37° C.) is observed over 30 minutes and the bleed time is determined.

### C) WORKING EXAMPLES OF PHARMACEUTICAL PREPARATIONS

The substances according to the invention can, for example, be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP) and 2 mg of magnesium stearate.

Tablet weight 212 mg. Diameter 8 mm, radius of curvature 12 mm.

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tabletting press (see above for format of the tablet).

### Oral Suspension:

### Composition:

1000 mg of the compound of Example 1, 1000 mg of ethanol (96%), 400 mg of Rhodigel and 99 g of water.

10 ml of oral suspension correspond to a single dose of 100 mg of the compound of the invention.

### Production:

The Rhodigel is suspended in ethanol, and the compound of Example 1 is added to the suspension. The water is added while stirring. The mixture is stirred for about 6 h until swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or polyethylene glycol 400/water 30% m/m). The solution is subjected to sterile filtration and dispensed into sterile and pyrogen-free injection vessels.

1. A compound of the formula 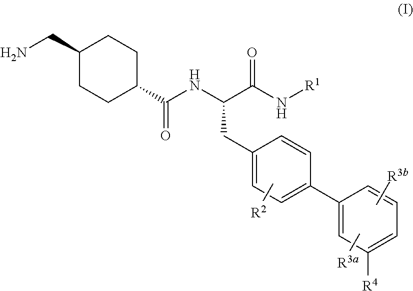
in which
R¹ represents a group of the formula 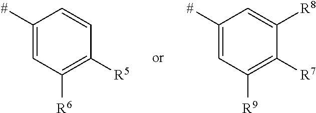
where # is the point of attachment to the nitrogen atom,
R⁵ represents 5-membered heteroaryl,
where heteroaryl may be substituted by a substituent selected from the group consisting of oxo, chlorine, cyano, hydroxyl and C₁-C₃-alkyl,
in which alkyl may be substituted by 1 to 3 substituents selected independently from the group consisting of hydroxy, amino, hydroxycarbonyl and methoxy, or
in which alkyl may be substituted by 1 to 7 fluorine substituents, or
in which alkyl is substituted by a substituent selected from the group consisting of hydroxy, amino, hydroxycarbonyl and methoxy, and in which alkyl is additionally substituted by 1 to 6 fluorine substituents,
R⁶ represents hydrogen, fluorine or chlorine,
R⁷ and R⁸ together with the carbon atoms to which they are attached form a 5-membered heterocycle,
where the heterocycle may be substituted by 1 to 2 substituents selected independently from the group consisting of oxo, chlorine, cyano, hydroxyl, C₁-C₃-alkyl, pyrazolyl and pyridyl,
in which alkyl may be substituted by 1 to 3 substituents selected independently from the group consisting of hydroxy, amino, hydroxycarbonyl and methoxy, or
in which alkyl may be substituted by 1 to 7 fluorine substituents, or
in which alkyl is substituted by a substituent selected from the group consisting of hydroxy, amino, hydroxycarbonyl and methoxy, and in which alkyl is additionally substituted by 1 to 6 fluorine substituents,
R⁹ represents hydrogen, fluorine or chlorine,
R² represents hydrogen, fluorine, chlorine, methyl or methoxy,
R^{3a} represents hydrogen, fluorine, chlorine, C₁-C₄-alkyl, methoxy or trifluoromethyl,
R^{3b} represents hydrogen or methyl,
R⁴ represents amino, cyano, trifluoromethyl, C₁-C₃-alkoxy, C₁-C₃-alkylamino, C₁-C₃-alkoxycarbonyl, —S(O)₂NR¹⁰R¹¹, —C(O)NR¹²R¹³ or —NR¹⁴(CO)R¹⁵,
where alkoxy is substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
in which n is a number from 1 to 6,
in which m is a number from 1 to 6, and
where
R¹⁰ represents hydrogen, C₁-C₃-alkyl, C₃-C₆-cycloalkyl, benzyl or 4- to 8-membered heterocyclyl which is attached via a carbon atom,
R¹¹ represents hydrogen or C₁-C₃-alkyl, or
R¹⁰ and R¹¹ together with the nitrogen atom to which they are attached form a 4- to 7-membered heterocycle,
in which the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R¹² represents hydrogen, C₁-C₃-alkyl, C₁-C₃-alkoxy, C₃-C₆-cycloalkyl, benzyl or 4- to 8-membered heterocyclyl which is attached via a carbon atom,
in which alkyl may be substituted by 1 to 2 substituents independently of one another selected from the group consisting of fluorine, hydroxy, amino, hydroxycarbonyl, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
in which n is a number from 1 to 6,
in which m is a number from 1 to 6, and
in which cycloalkyl may be substituted by 1 to 2 substituents independently of one another selected from the group consisting of oxo, fluorine, hydroxy, amino, C₁-C₄-alkyl and C₁-C₃-alkylamino,
in which alkyl and alkylamino for their part may be substituted by 1 to 5 fluorine substituents, and
in which heterocyclyl may be substituted by 1 to 2 substituents independently of one another selected from the group consisting of oxo, fluorine, hydroxy, amino, hydroxycarbonyl, C₁-C₄-alkyl, C₁-C₃-alkylamino, C₁-C₄-alkoxycarbonyl, aminocarbonyl and C₁-C₃-alkyl aminocarbonyl,
in which alkyl and alkylamino for their part may be substituted by 1 to 5 fluorine substituents, and
in which heterocyclyl may additionally be substituted by 1 to 4 substituents independently of one another selected from the group consisting of fluorine and methyl,
R¹³ represents hydrogen or C₁-C₃-alkyl, or
R¹² and R¹³ together with the nitrogen atom to which they are attached form a 4- to 7-membered heterocycle,
in which the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
in which alkyl for its part may be substituted by a hydroxy substituent,
R¹⁴ represents hydrogen or C₁-C₃-alkyl,
R¹⁵ represents C₁-C₄-alkyl, C₃-C₆-cycloalkyl, phenyl or 5- to 7-membered heterocyclyl,
or one of the salts thereof, solvates thereof or solvates of the salts thereof.

2. The compound according to claim 1, characterized in that
R¹ represents a group of the formula 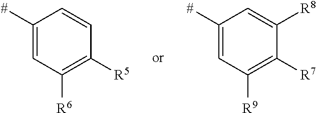
where # is the point of attachment to the nitrogen atom,
R⁵ represents 5-membered heteroaryl,
where heteroaryl may be substituted by a substituent selected from the group consisting of oxo, chlorine and C₁-C₃-alkyl,
in which alkyl may be substituted by 1 to 2 substituents selected independently from the group consisting of hydroxycarbonyl and methoxy, or
in which alkyl may be substituted by 1 to 7 fluorine substituents, or
in which alkyl is substituted by a hydroxycarbonyl substituent and in which alkyl is additionally substituted by 1 to 6 fluorine substituents,
R⁶ represents hydrogen or fluorine,
R⁷ and R⁸ together with the carbon atoms to which they are attached form a 5-membered heterocycle,
where the heterocycle may be substituted by 1 to 2 substituents selected independently from the group consisting of oxo, chlorine, hydroxyl, C₁-C₃-alkyl, pyrazolyl and pyridyl,
in which alkyl may be substituted by 1 to 2 substituents selected independently from the group consisting of hydroxycarbonyl and methoxy, or
in which alkyl may be substituted by 1 to 7 fluorine substituents, or
in which alkyl is substituted by a hydroxycarbonyl substituent and in which alkyl is additionally substituted by 1 to 6 fluorine substituents,
R⁹ represents hydrogen or fluorine,
R² represents hydrogen, fluorine, methyl or methoxy,
R^{3a} represents hydrogen, fluorine, chlorine, C₁-C₄-alkyl, methoxy or trifluoromethyl,
R^{3b} represents hydrogen,
R⁴ represents amino, cyano, trifluoromethyl, C₁-C₃-alkoxy, C₁-C₃-alkylamino, C₁-C₃-alkoxycarbonyl, —S(O)₂NR¹⁰R¹¹, —C(O)NR¹²R¹³ or —NR¹⁴(CO)R¹⁵,
where alkoxy is substituted by a morpholinyl substituent, and
where
R¹⁰ represents hydrogen, methyl, ethyl, C₃-C₆-cycloalkyl or 4- to 8-membered heterocyclyl which is attached via a carbon atom,
R¹¹ represents hydrogen, methyl or ethyl, or
R¹⁰ and R¹¹ together with the nitrogen atom to which they are attached form a 4- to 7-membered heterocycle,
in which the heterocycle may be substituted by 1 to 2 substituents independently of one another selected from the group consisting of C₁-C₄-alkyl,
R¹² is hydrogen, C₁-C₃-alkyl, C₃-C₆-cycloalkyl, or 4- to 8-membered heterocyclyl bonded via a carbon atom,
in which alkyl may be substituted by 1 to 2 substituents independently of one another selected from the group consisting of C₁-C₃-alkylamino and morpholinyl, and
in which cycloalkyl may be substituted by 1 to 2 substituents independently of one another selected from the group consisting of hydroxy, amino, C₁-C₄-alkyl and C₁-C₃-alkylamino, and
in which heterocyclyl may be substituted by 1 to 2 substituents independently of one another selected from the group consisting of C₁-C₄-alkyl,
R¹³ represents hydrogen, methyl or ethyl, or
R¹² and R¹³ together with the nitrogen atom to which they are attached form a 4- to 7-membered heterocycle,
in which the heterocycle may be substituted by 1 to 2 substituents independently of one another selected from the group consisting of C₁-C₄-alkyl,
R¹⁴ represents hydrogen, methyl or ethyl,
R¹⁵ represents C₁-C₄-alkyl or 5- to 7-membered heterocyclyl,
or one of the salts thereof, solvates thereof or solvates of the salts thereof.

3. The compound according to claim 1, characterized in that
R¹ represents a group of the formula 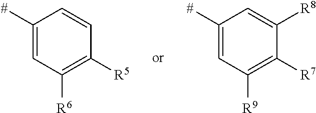
where # is the point of attachment to the nitrogen atom,
R⁵ represents 5-membered heteroaryl,
where heteroaryl may be substituted by a chlorine substituent,
R⁶ represents hydrogen,
R⁷ and R⁸ together with the carbon atoms to which they are attached form a 5-membered heterocycle,
where the heterocycle may be substituted by an oxo substituent,
R⁹ represents hydrogen,
R² represents hydrogen,
R^{3a} represents hydrogen, fluorine, C₁-C₄-alkyl or methoxy,
R^{3b} represents hydrogen,
R⁴ represents cyano, trifluoromethyl, C₁-C₃-alkoxy, C₁-C₃-alkylamino, C₁-C₃-alkoxycarbonyl, —S(O)₂NR¹⁰R¹¹, —C(O)NR¹²R¹³ or —NR¹⁴(CO)R¹⁵,
where alkoxy is substituted by a morpholinyl substituent, and
where
R¹⁰ represents hydrogen or methyl,
R¹¹ represents hydrogen or methyl, or
R¹⁰ and R¹¹ together with the nitrogen atom to which they are attached form a 4- to 7-membered heterocycle,
R¹² represents C₁-C₃-alkyl or 4- to 8-membered heterocyclyl which is attached via a carbon atom,
in which alkyl may be substituted by 1 to 2 substituents independently of one another selected from the group consisting of C₁-C₃-alkylamino and morpholinyl,
R¹³ represents hydrogen, or
R¹² and R¹³ together with the nitrogen atom to which they are attached form a 4- to 7-membered heterocycle,
in which the heterocycle may be substituted by 1 to 2 methyl substituents,
R¹⁴ represents hydrogen,
R¹⁵ represents 5- to 7-membered heterocyclyl,
or one of the salts thereof, solvates thereof or solvates of the salts thereof.

4. The compound of claim 1, characterized in that
R¹ represents a group of the formula 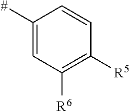
where # is the point of attachment to the nitrogen atom,
R⁵ is triazolyl or tetrazolyl,
where triazolyl may be substituted by a chlorine substituent,
R⁶ represents hydrogen, or
R¹ represents 2,3-dihydro-1H-indazol-6-yl or 2,3-dihydro-1H-benzimidazol-5-yl,
where 2,3-dihydro-1H-indazol-6-yl and 2,3-dihydro-1H-benzimidazol-5-yl may be substituted by an oxo substituent,
R² represents hydrogen,
R^{3a} represents hydrogen, fluorine, C₁-C₄-alkyl or methoxy,
R^{3b} represents hydrogen,
R⁴ represents cyano, trifluoromethyl, C₁-C₃-alkoxycarbonyl, —S(O)₂NR¹⁰R¹¹ or —C(O)NR¹²R¹³,
where
R¹⁰ represents hydrogen or methyl,
R¹¹ represents hydrogen or methyl, or
R¹⁰ and R¹¹ together with the nitrogen atom to which they are attached form a morpholinyl or piperazinyl,
R¹² represents C₁-C₃-alkyl or heterocyclyl which is attached via a carbon atom and selected from the group consisting of pyrrolidinyl and piperidinyl,
in which alkyl may be substituted by 1 to 2 substituents independently of one another selected from the group consisting of C₁-C₃-alkylamino and morpholinyl,
R¹³ represents hydrogen, or
R¹² and R¹³ together with the nitrogen atom to which they are attached form a morpholinyl or piperazinyl,
in which morpholinyl and piperazinyl may be substituted by 1 to 2 methyl substituents,
or one of the salts thereof, solvates thereof or solvates of the salts thereof.

5. A method for preparing the compound of claim 1 of the formula (I) or one of the salts thereof, solvates thereof or solvates of the salts thereof, characterized in that a compound of the formula 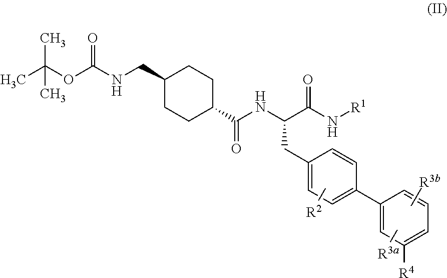
in which R¹, R², R^{3a}, R^{3b} and R⁴ have the meaning given in claim 1, is reacted with an acid.

6. A method for the treatment and/or prophylaxis of diseases using the compound of claim 1.

7. A method of making a medicament for the treatment and/or prophylaxis of diseases using the compound of claim 1.

8. A method of making a medicament for the treatment and/or prophylaxis of thrombotic or thromboembolic disorders using the compound of claim 1.

9. A medicament comprising the compound according to claim 1 in combination with an inert, nontoxic, pharmaceutically suitable excipient.

10. A method for treatment and/or prophylaxis of thrombotic or thromboembolic disorders using the medicament of claim 9.

11. A method for the treatment of thrombotic or thromboembolic disorders in humans and animals by administration of a therapeutically effective amount of the compound of claim 1.

12. A method for the treatment of thrombotic or thromboembolic disorders in humans and animals by administration of a therapeutically effective amount of the medicament of claim 9.

13. A method for the treatment of thrombotic or thromboembolic disorders in humans and animals by administration of a therapeutically effective amount of the medicament of claim 7.
